(19) United States
(12) Patent Application Publication
(10) Pub. No.: US 2010/0120105 A1
(43) Pub. Date: May 13, 2010

(54) CARBON PATHWAY OPTIMIZED PRODUCTION HOSTS FOR THE PRODUCTION OF ISOBUTANOL

(75) Inventors: Larry Cameron Anthony, Aston, PA (US); Michael Dauner, Claymont, DE (US); Gail K. Donaldson, Newark, DE (US); Brian James Paul, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: BUTAMAX (TM) ADVANCED BIOFUELS LLC, Wilmington, DE (US)

(21) Appl. No.: 12/606,410

(22) Filed: Oct. 27, 2009

Related U.S. Application Data
(60) Provisional application No. 61/108,680, filed on Oct. 27, 2008.
(60) Provisional application No. 61/108,684, filed on Oct. 27, 2008.
(60) Provisional application No. 61/108,689, filed on Oct. 27, 2008.

(51) Int. Cl. C12P 7/04; C12N 1/00; C12N 1/21; C12N 1/19

(52) U.S. Cl. 435/157; 435/243; 435/252.3; 435/252.33; 435/252.34; 435/252.31; 435/254.23; 435/254.22; 435/254.21; 435/254.2

(57) ABSTRACT

A microbial host cell is provided for the production of isobutanol. Carbon flux in the cell is optimized through the Entner-Doudoroff pathway.

### CROSS-REFERENCE TO RELATED APPLICATIONS

This application is related to and claims the benefit of U.S. Provisional Application Nos. 61/108,680; 61/108,684; and 61/108,689, all filed on Oct. 27, 2008, the disclosures of which are hereby incorporated by reference in their entirety.

### FIELD OF THE INVENTION

The invention relates to the field of industrial microbiology. More specifically a microbial production host for the production of isobutanol is provided wherein the host is genetically modified to maximize carbon flux through the Entner-Doudoroff pathway.

### BACKGROUND OF THE INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Methods for the chemical synthesis of isobutanol are known, such as oxo synthesis, catalytic hydrogenation of carbon monoxide (Ullmann's Encyclopedia of Industrial Chemistry, 6^{th} edition, 2003, Wiley-VCHVerlag GmbH and Co., Weinheim, Germany, Vol. 5, pp. 716-719) and Guerbet condensation of methanol with n-propanol (Carlini et al., J. Mol. Catal. A: Chem. 220:215-220 (2004)). These processes use starting materials derived from petrochemicals and are generally expensive and are not environmentally friendly. The production of isobutanol from plant-derived raw materials would minimize green house gas emissions and would represent an advance in the art.

U.S. Patent Application Publication No. 20070092957 describes a variety of production hosts and methods for the biological production of isobutanol.

Recently Atsumi, S., et al., (Nature, 451:86-90, 2008) described development of a recombinant E. coli strain which produced isobutanol in concentrations up to 300 mM. This recombinant E. coli was disrupted in genes adhE, IdhA, frdBC, fnr, pta and pflB and contained two plasmids bearing an isobutanol biosynthetic pathway similar to that described in U.S. Patent Application Publication No. 20070092957. These plasmids carried an acetolactate synthase, an acetohydroxy acid reductoisomerase, an acetohydroxy acid dehydratase, a 2-keto acid decarboxylase and an alcohol dehydrogenase

Enzymatic pathways useful for the production of isobutanol have specific co-factor requirements. Certain of these have the need for one NADH and one NADPH for every 2 molecules of pyruvate processed in the pathway to isobutanol. In many microbial systems glucose is metabolized to pyruvate via one of three glycolytic pathways known as the Entner-Doudoroff pathway (EDP), the oxidative pentose phosphate pathway (oxidative PPP) and the Embden-Meyerhof pathway (EMP). One of the challenges in designing a production host that efficiently produces isobutanol is to optimize pyruvate production from glycolytic pathways so that the co-factor requirements of the isobutanol biosynthetic pathway are met. Neither the oxidative pentose phosphate pathway nor the EMP typically produces the required co-factor balance. However, glucose metabolized via the EDP can produce one NADH and one NADPH for every 2 molecules of pyruvate.

Yeast has been transformed to express phosphogluconate dehydratase and 2-keto-3-deoxygluconate-6-phosphate aldolase to allow fermentation of sugar via the Entner-Doudoroff pathway (EDP). The use of such genetically modified yeast for use in alcoholic fermentations such as beer, cider, wine was disclosed in Publication WO1995025799A1 and U.S. Pat. No. 5,786,186. Production of an L-amino acid by a Gram negative bacterium also was increased by overexpressing the 6-phosphogluconate dehydratase and 2-keto-3-deoxy-6-phosphogluconate aldolase enzymes of the EDP in U.S. Pat. No. 7,037,690.

It would be an advance in the art to provide an isobutanol producing host having carbon flux optimized through the EDP, however, there are no reports of such flux considerations in prior art.

### SUMMARY OF THE INVENTION

Provided herein are recombinant microbial host cells comprising a functional or enhanced EDP and an isobutanol production pathway wherein said functional or enhanced EDP provides for increased isobutanol production as compared to the same host cell without said functional or enhanced EDP.

Also provided herein are microbial host cells wherein the functional or enhanced EDP is provided by expression of one or more heterologous genes that encode functional EDP pathway enzymes or up-regulation of one or more endogenous genes that encode enhanced EDP pathway enzymes, or both, and one or more modification to said host cell that provides for increased carbon flux through the EDP or reducing equivalents balance such that the cofactors produced during the conversion of glucose to pyruvate are matched with the cofactors required for the conversion of pyruvate to isobutanol, or both, whereby isobutanol production is increased as compared to the same host cell without said one or more modification that provides for increased carbon flux through the EDP or reducing equivalents balance, or both. In some embodiments, said one or more modification to said host cell that provides for increased carbon flux through EDP or reducing equivalents balance, or both, is one or more genetic modification selected from the group consisting of: a) a disruption in the expression of at least one enzyme of the EMP; b) a disruption in the expression of at least one enzyme of the PPP; and c) a modification in any one of EDP, EMP, or PPP such that cofactors produced during the conversion of glucose to pyruvate are matched with the cofactors required for the conversion of pyruvate to isobutanol.

Microbial host cells provided herein can further comprise: i) at least one gene encoding acetolactate synthase for the conversion of pyruvate to acetolactate; ii) at least one gene encoding ketol acid reductoisomerase for the conversion of acetolactate to 2,3-dihydroxyisovalerate; iii) at least one gene encoding an acetohydroxy acid dehydratase for the conversion of 2,3-dihydroxyisovalerate to α-ketoisovalerate; iv) at least one gene encoding valine dehydrogenase or transaminase for the conversion of α-ketoisovalerate to valine; v) at least one gene encoding a valine decarboxylase for the conversion of valine to isobutylamine; vi) at least one gene encoding an omega transaminase for the conversion of isobutylamine to isobutyraldehyde, and (vii) at least one gene encoding a branched chain alcohol dehydrogenase for the conversion of isobutyraldehyde to isobutanol.

Microbial host cells provided herein can further comprise: i) at least one gene encoding acetolactate synthase for the conversion of pyruvate to acetolactate; ii) at least one gene encoding ketol acid reductoisomerase for the conversion of acetolactate to 2,3-dihydroxyisovalerate; iii) at least one gene encoding acetohydroxy acid dehydratase for the conversion of 2,3-dihydroxyisovalerate to α-ketoisovalerate; iv) at least one gene encoding a branched chain ketoacid dehydrogenase for the conversion of α-ketoisovalerate to isobutyryl-CoA; v) at least one gene encoding an acylating aldehyde dehydrogenase for the conversion of isobutyryl-CoA to isobutyraldehyde; and vi) at least one gene encoding a branched chain aldehyde dehydrogenase for the conversion of isobutyraldehyde to isobutanol.

Microbial host cells provided herein can further comprise: i) at least one gene encoding acetolactate synthase for the conversion of pyruvate to acetolactate; ii) at least one gene encoding acetohydroxy acid reductoisomerase for the conversion of acetolactate to 2,3-dihydroxyisovalerate; iii) at least one gene encoding acetohydroxy acid dehydratase for the conversion of 2,3-dihydroxyisovalerate to α-ketoisovalerate; iv) at least one gene encoding branched-chain a-keto acid decarboxylase for the conversion of α-ketoisovalerate to isobutyraldehyde; and v) at least one gene encoding branched-chain alcohol dehydrogenase for the conversion of isobutyraldehyde to isobutanol.

In some embodiments, the functional or enhanced EDP is provided by expression of at least one recombinant DNA molecule encoding an enzyme of the EDP selected from the group consisting of a) glucose-6-phosphate dehydrogenase; b) 6-phosphogluconolactonase; c) phosphogluconate dehydratase and d) 2-dehydro-3-deoxyphosphogluconate aldolase.

In some embodiments the disruption in expression of at least one enzyme of the EMP is a disruption in expression of at least one enzyme selected from the group consisting of: a) 6-phosphofructokinase, b) fructose-bisphosphate aldolase and c) glucose-6-phosphate isomerase.

In some embodiments, the host cell is a member of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Klebsiella, Paenibacillus, Arthrobacter, Corynebacterium, Brevibacterium, Schizosaccharomyces, Kluyveromyces, Yarrowia, Pichia, Candida, Hansenula, or Saccharomyces. In some embodiments the host cell is E. coli, S. cerevisiae, or L. plantarum. In some embodiments, the host cell is E. coli and wherein the host cell further comprises downregulation or deletion of soluble transhydrogenase activity.

In some embodiments the host cell comprises a disruption in at least one of the following genes: pfk1, pfk2, fba1, gnd1, gnd2, pgi, pfkA, pfkB, fbaA, fbaB, gnd, pgi, sthA, PGI1, PFK1, PFK2, FBA1, GND1, or GND2.

In some embodiments the host cell is S. cerevisiae and the PFK1 gene encodes 6-phosphofurctokinase having the amino acid sequence as set forth in SEQ ID NO: 172; the PFK2 gene encodes a 6-phosphofructokinase having the amino acid sequence as set forth in SEQ ID NO: 174; the FBA1 gene encodes a fructose-bisphosphate aldolase having the amino acid sequence as set forth in SEQ ID NO: 186; the GND1 gene encodes a 6-phosphogluconate dehydrogenase having the amino acid sequence as set forth in SEQ ID NO: 148; and the PGI1 gene encodes a glucose-6-phosphate isomerase having the amino acid sequence as set forth in SEQ ID NO: 160. In some embodiments, the host cell is L. plantarum and the pfkA gene encodes a 6-phosphofructokinase having the amino acid sequence as set forth in SEQ ID NO:176; the fba gene encodes a fructose-bisphosphate aldolase having the amino acid sequence as set forth in SEQ ID NO:188; the gnd1 gene encodes a 6-phosphogluconate dehydrogenase having the amino acid sequence as set forth in SEQ ID NO:152; the gnd2 gene encodes a 6-phosphogluconate dehydrogenase having the amino acid sequence as set forth in SEQ ID NO:154; and the pgi gene encodes a glucose-6-phosphate isomerase having the amino acid sequence as set forth in SEQ ID NO:162. In some embodiments, the host cell comprises a heterologous glucose-6-phosphate dehydrogenase gene encoding a polypeptide having the amino acid sequence as set forth in SEQ ID NO:128. In some embodiments, any endogenous gene encoding a polypeptide having glucose-6-phosphate dehydrogenase activity has been disrupted or deleted. In some embodiments, the host cell comprises a 6-phosphogluconolactonase gene encoding a polypeptide having the amino acid sequence as set forth in SEQ ID NO:106.

Provided herein are recombinant microbial host cells comprising an isobutanol production pathway and at least one of the following: a) at least one recombinant DNA molecule encoding an enzyme of the EDP; b) a disruption in the expression of at least one enzyme of the EMP; or c) a disruption in the expression of at least one enzyme of the PPP; wherein production of isobutanol by said host cell is enhanced by at least 10% as compared to the same host cell without one of (a)-(c).

Also provided are methods for improved production of isobutanol comprising contacting a microbial host cell provided herein with a fermentable carbon substrate for a time sufficient for isobutanol to be produced. In some embodiments, the fermentable carbon substrate is from lignocellulosic biomass and comprises one or more sugars selected from the group consisting of glucose, fructose, sucrose, xylose and arabinose. In some embodiments, the relative flux through at least one reaction unique to the EDP is at least 1% greater than that in the same host cell without functional or enhanced EDP. In some embodiments, the relative flux through at least one reaction unique to the EDP is enhanced by at least about 10%. In some embodiments the yield of isobutanol is greater than about 0.3 g/g.

Provided herein are methods for the production of isobutanol comprising a) providing a microbial host cell as provided herein; and b) contacting the host cell with a fermentable carbon substrate under anaerobic conditions. In some embodiments, the host cell is E. coli and endogenous pyruvate formate lyase, fumarate reductase, alcohol dehydrogenase, and lactate dehydrogenase activities are downregulated or disrupted.

In some embodiments, the yield of isobutanol is greater than or equal to about 0.3 g/g, in some embodiments, the yield of isobutanol is greater than or equal to about 0.35 g/g, and in some embodiments, the yield of isobutanol is greater than or equal to about 0.39 g/g.

In some embodiments, the host cell is S. cerevisiae and endogenous pyruvate decarboxylase activity is downregulated or disrupted. In some embodiments, the host cell is L. plantarum and wherein endogenous lactate dehydrogenase activity is downregulated or disrupted.

### BRIEF DESCRIPTION OF THE FIGURES AND SEQUENCE DESCRIPTIONS

FIG. 1 depicts isobutanol biosynthetic pathways.

FIG. 2 depicts the interaction between the EDP, the oxidative PPP and the EMP.

FIG. 3 illustrates genes that can be up-regulated (circled) or down-regulated (crossed out) to enhance the EDP in an E. coli host cell.

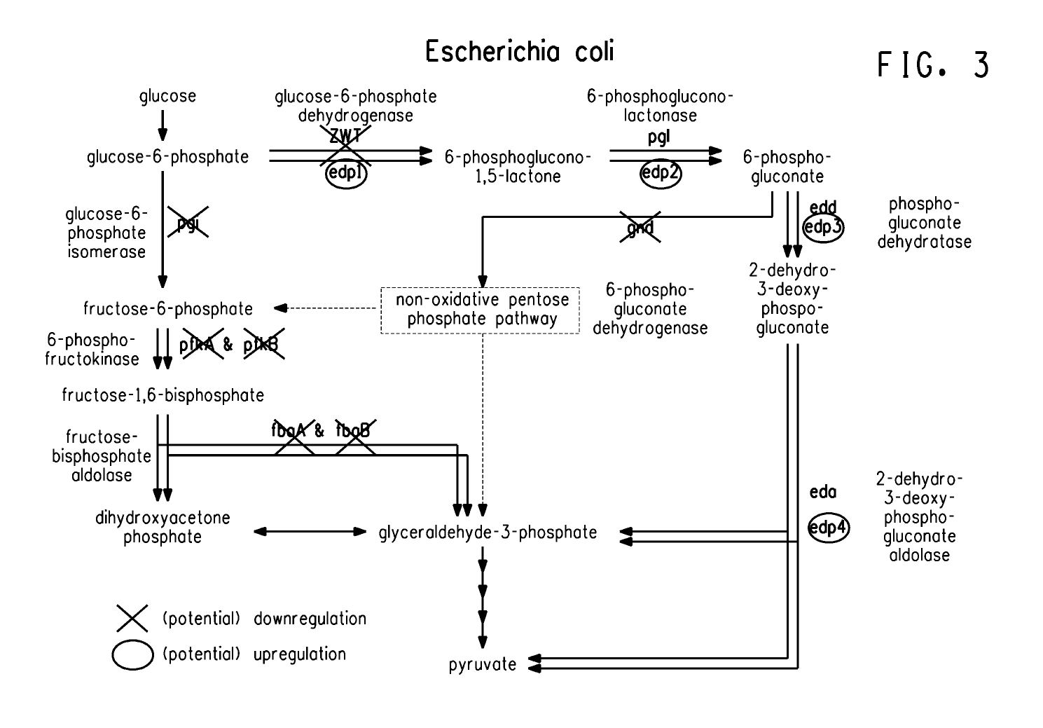

FIG. 4 illustrates genes that can be up-regulated (circled) or down-regulated (crossed out) to enhance the EDP in Saccharmoyces cerevisae host cell.

FIG. 5 illustrates genes that can be up-regulated (circled) or down-regulated (crossed out) to enhance the EDP in Lactobacillus plantarum host

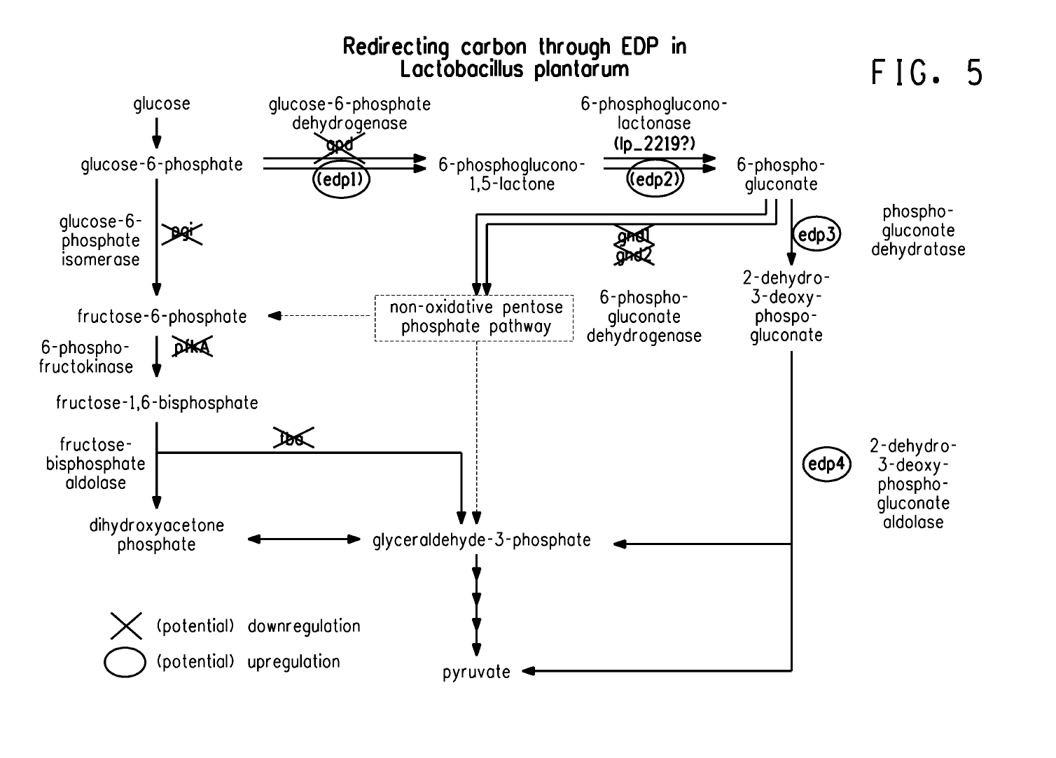

The following sequences conform with 37C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST. 25 (1998) and the sequence listing requirements of the EPO and PCT (Rules 5.2 and 49.5 (a-bis), and Section 208 and Annex C of the Administrative Instructions). The symbols and format used for nucleotide and amino acid sequence data comply with the rules set forth in 37C.F.R. §1.822.

TABLE 1
SEQ ID NOs of the Genes and Proteins of
Various Isobutanol Pathways

| | SEQ ID NO: | SEQ ID |
|---|---|---|
| | Nucleic | NO: |
| Description | acid | Peptide |
| Bacillus subtilis alsS (acetolactate synthase) | 1 | 2 |
| Bacillus subtilis alsS (acetolactate synthase), | 254 | 2 |
| codon optimized | | |
| Klebsiella pneumoniae budB (acetolactate | 3 | 4 |
| synthase) | | |
| Lactococcus lactis als (acetolactate synthase) | 5 | 6 |
| Escherichia coli ilvC (acetohydroxy acid | 7 | 8 |
| reductoisomerase) | | |
| S. cerevisiae ILV5 (acetohydroxy acid | 9 | 10 |
| reductoisomerase) | | |
| Methanococcus maripaludis ilvC (Ketol-acid | 11 | 12 |
| reductoisomerase) | | |
| B. subtilis ilvC (acetohydroxy acid | 13 | 14 |
| reductoisomerase) | | |
| E. coli ilvD (acetohydroxy acid dehydratase) | 15 | 16 |
| S. cerevisiae ILV3 (Dihydroxyacid dehydratase) | 17 | 18 |
| M. maripaludis ilvD (Dihydroxy-acid | 19 | 20 |
| dehydratase) | | |
| B. subtilis ilvD (dihydroxy-acid dehydratase) | 21 | 22 |
| Lactococcus lactis kdcA (branched-chain alpha- | 23 | 24 |
| ketoacid decarboxylase) | | |
| Lactococcus lactis kivD (branched-chain α-keto | 25 | 26 |
| acid decarboxylase), codon optimized | | |
| Lactococcus lactis kivD (branched-chain α-keto | 189 | 26 |
| acid decarboxylase) | | |
| Salmonella typhimurium (indolepyruvate | 27 | 28 |
| decarboxylase) | | |
| Clostridium acetobutylicum pdc (Pyruvate | 29 | 30 |
| decarboxylase) | | |
| Saccharomyces cerevisiae YPR1 (2- | 31 | 32 |
| methylbutyraldehyde reductase) | | |
| S. cerevisiae ADH6 (NADPH-dependent | 33 | 34 |
| cinnamyl alcohol dehydrogenase) | | |
| E. coli yqhD (branched-chain alcohol | 35 | 36 |
| dehydrogenase) | | |
| Clostridium acetobutylicum bdhA (NADH- | 37 | 38 |
| dependent butanol dehydrogenase A) | | |
| Clostridium acetobutylicum bdhB Butanol | 39 | 40 |
| dehydrogenase | | |
| Bacillus subtilis bkdAA(branched-chain keto | 41 | 42 |
| acid dehydrogenase E1 subunit) | | |
| B. subtilis bkdAB (branched-chain alpha-keto | 43 | 44 |
| acid dehydrogenase E1 subunit) | | |
| B. subtilis bkdB (branched-chain alpha-keto acid | 45 | 46 |
| dehydrogenase E2 subunit) | | |
| B. subtilis lpdV (branched-chain alpha-keto acid | 47 | 48 |
| dehydrogenase E3 subunit) | | |
| Pseudomonas putida bkdA1 (keto acid | 49 | 50 |
| dehydrogenase E1-alpha subunit) | | |
| P. putida bkdA2 (keto acid dehydrogenase E1- | 51 | 52 |
| beta subunit) | | |
| P. putida bkdB (transacylase E2) | 53 | 54 |
| P. putida 1pdV (lipoamide dehydrogenase) | 55 | 56 |
| Clostridium beijerinckii ald (coenzyme A | 57 | 58 |
| acylating aldehyde dehydrogenase) | | |
| C. acetobutylicum adhe1(aldehyde | 59 | 60 |
| dehydrogenase) | | |
| C. acetobutylicum adhe (alcohol-aldehyde | 61 | 62 |
| dehydrogenase) | | |
| P. putida nahO (acetaldehyde dehydrogenase) | 63 | 64 |
| Thermus thermophilus (acetaldehyde | 65 | 66 |
| dehydrogenase) | | |
| E. coli avtA (valine-pyruvate transaminase) | 67 | 68 |
| B. licheniformis avtA (valine-pyruvate | 69 | 70 |
| transaminase) | | |
| E. coli ilvE (branched chain amino acid | 71 | 72 |
| aminotransferase) | | |
| S. cerevisiae BAT2 (branched chain amino acid | 73 | 74 |
| aminotransferase) | | |
| Methanobacterium thermoautotrophicum | 75 | 76 |
| (branched chain amino acid aminotransferase) | | |
| Streptomyces coelicolor (valine dehydrogenase) | 77 | 78 |
| B. subtilis bcd (leucine dehydrogenase) | 79 | 80 |
| Streptomyces viridifaciens (valine decarboxyase) | 81 | 82 |
| Alcaligenes denitrificans aptA (omega-amino | 83 | 84 |
| acid:pyruvate transaminase) | | |
| Ralstonia eutropha (alanine-pyruvate | 85 | 86 |
| transaminase) | | |
| Shewanella oneidensis (beta alanine-pyruvate | 87 | 88 |
| transaminase) | | |
| P. putida (beta alanine-pyruvate transaminase) | 89 | 90 |
| Streptomyces cinnamonensis icm (isobutyrl-CoA | 91 | 92 |
| mutase) | | |
| S. cinnamonensis icmB (isobutyrl-CoA mutase) | 93 | 94 |
| S. coelicolor SCO5415 (isobutyrl-CoA mutase) | 95 | 96 |
| S. coelicolor SCO4800 (isobutyrl-CoA mutase) | 97 | 98 |
| Streptomyces avermitilis icmA (isobutyrl-CoA | 99 | 100 |
| mutase) | | |
| S. avermitilis icmB (isobutyrl-CoA mutase) | 101 | 102 |
| Achromobacter xyloxidans sadB (butanol | 103 | 104 |
| dehydrogenase) | | |
| Vibrio cholera (KARI) | 212 | 213 |
| Pseudomonas aeruginosa PAO1 (KARI) | 214 | 215 |
| Pseudomonas fluorescens PF5 (KARI) | 216 | 217 |
| Saccharomyces cerevisiae (ILV3 gene) | 7 | — |
| Saccharomyces cerevisiae (ILV5 gene) | 9 | — |
| Lactococcus lactis subsp. lactis, ilvD | 109 | 110 |
| (dihydroxyacid dehydratase) | | |
| Bacillus subtilis ilvC (ketol-acid | 251 | 14 |
| reductoisomerase), codon optimized | | |

TABLE 2
List of SEQ ID Numbers for
Genes and Proteins of Various Reactions of the EDP

| | SEQ ID NO: | SEQ ID |
|---|---|---|
| | Nucleic | NO: |
| Description | acid | Peptide |
| Aspergillus niger | 117 | 118 |
| gsdA(glucose-6-phosphate dehydrogenase) | | |
| Aspergillus nidulans FGSC A4 locus_tag = | 119 | 120 |
| “AN2981.2 (glucose-6-phosphate dehydrogenase) | | |
| Schizosaccharomyces pombe 972h- | 123 | 122 |
| locus_tag = “SPCC794.01c”, chromosome III | | |
| (glucose-6-phosphate dehydrogenase) | | |
| Schizosaccharomyces pombe 972h- | 124 | 125 |
| locus_tag = “SPAC3C7.13c”, chromosome I | | |
| (glucose-6-phosphate dehydrogenase) | | |
| Schizosaccharomyces pombe 972h- | 121 | 126 |
| zwf1, locus_tag = “SPAC3A12.18”, | | |
| chromosome I (glucose-6-phosphate | | |
| dehydrogenase) | | |
| Escherichia coli K12 MG1655 | 127 | 128 |
| zwf (glucose-6-phosphate dehydrogenase) | | |
| Lactobacillus plantarum WCFS1 | 131 | 132 |
| gpd (glucose-6-phosphate dehydrogenase) | | |
| Saccharomyces cerevisiae | 133 | 134 |
| ZWF1 (glucose-6-phosphate dehydrogenase) | | |
| Azotobacter vinelandii AvOP | 194 | 195 |
| locus_tags = “AvinDRAFT_4462”, | 196 | 197 |
| “AvinDRAFT_8258”, “AvinDRAFT_4842” and | 198 | 199 |
| “AvinDRAFT_0719” (2-dehydro-3-deoxy- | 200 | 201 |
| phosphogluconate aldolase) | | |
| Pseudomonas putida KT2440 | 202 | 203 |
| eda (2-dehydro-3-deoxy-phosphogluconate | | |
| aldolase) | | |
| Pseudomonas fluorescens Pf-5 | 204 | 205 |
| eda (2-dehydro-3-deoxy-phosphogluconate | | |
| aldolase) | | |
| Zymomonas mobilis ZM4 | 206 | 207 |
| eda (2-dehydro-3-deoxy-phosphogluconate | | |
| aldolase) | | |
| Escherichia coli K12 MG1655 | 208 | 209 |
| eda (2-dehydro-3-deoxy-phosphogluconate | | |
| aldolase) | | |
| Zymomonas mobilis ZM4 | 135 | 136 |
| edd (phosphogluconate dehydratase) | | |
| Pseudomonas putida KT2440 | 137 | 138 |
| edd (phosphogluconate dehydratase) | | |
| Escherichia coli K12 MG1655 | 139 | 140 |
| edd (phosphogluconate dehydratase) | | |
| Escherichia coli K-12 MG1655 pgl (6- | 105 | 106 |
| phosphogluconolactonase) | | |
| Saccharomyces cerevisiae | 107 | 108 |
| SOL4 (6-phosphogluconolactonase) | | |
| (NP_011764.1) | | |
| Saccharomyces cerevisiae | 190 | 191 |
| SOL3 (6-phosphogluconolactonase) | | |
| (NP_012033.2) | | |
| Lactobacillus plantarum WCFS1 lp_2219 (6- | 111 | 112 |
| phosphogluconolactonase) | | |
| Zymomonas mobilis mobilis ZM4 | 113 | 114 |
| pgl (6-phosphogluconolactonase) (AAV90102.1) | | |
| Zymomonas mobilis mobilis ZM4 | 113 | 114 |
| pgl (6-phosphogluconolactonase) (YP_163213.1) | | |

TABLE 3
List of SEQ ID Numbers for
Genes and Proteins of Various Reactions of the oxidative PPP

| | SEQ ID NO: | SEQ ID |
|---|---|---|
| | Nucleic | NO: |
| Description | acid | Peptide |
| Aspergillus niger | 117 | 118 |
| g6pdh (glucose-6-phosphate dehydrogenase) | | |
| Aspergillus nidulans FGSC A4 | 119 | 120 |
| locus_tag = “AN2981.2 | | |
| (glucose-6-phosphate dehydrogenase) | | |
| Schizosaccharomyces pombe 972h- | 123 | 122 |
| locus_tag = “SPCC794.01c”, chromosome III | | |
| (glucose-6-phosphate dehydrogenase) | | |
| Schizosaccharomyces pombe 972h- | 124 | 125 |
| locus_tag = “SPAC3C7.13c”, chromosome I | | |
| (glucose-6-phosphate dehydrogenase) | | |
| Schizosaccharomyces pombe 972h- | 121 | 126 |
| zwf1, locus_tag = “SPAC3A12.18”, | | |
| chromosome I (glucose-6-phosphate | | |
| dehydrogenase) | | |
| Escherichia coli K12 MG1655 | 127 | 128 |
| zwf (glucose-6-phosphate dehydrogenase) | | |
| Lactobacillus plantarum WCFS1 | 131 | 132 |
| gpd (glucose-6-phosphate dehydrogenase) | | |
| Saccharomyces cerevisiae | 133 | 134 |
| ZWF1 (glucose-6-phosphate dehydrogenase) | | |
| Escherichia coli K-12 MG1655 | 105 | 106 |
| pgl (6-phosphogluconolactonase) | | |
| Saccharomyces cerevisiae | 107 | 108 |
| SOL4 (6-phosphogluconolactonase) | | |
| (NP_011764.1) | | |
| Saccharomyces cerevisiae | 190 | 191 |
| SOL3 (6-phosphogluconolactonase) | | |
| (NP_012033.2) | | |
| Lactobacillus plantarum WCFS1 | 111 | 112 |
| lp_2219 (6-phosphogluconolactonase) | | |
| Zymomonas mobilis mobilis ZM4 | 113 | 114 |
| pgl (6-phosphogluconolactonase) (AE008692.1) | | |
| Zymomonas mobilis mobilis ZM4 | 113 | 114 |
| pgl (6-phosphogluconolactonase) | | |
| (YP_163213.1) | | |
| Escherichia coli K12 MG1655 | 143 | 144 |
| gnd (6-phosphogluconate dehydrogenase) | | |
| Saccharomyces cerevisiae | 147 | 148 |
| GND2 (6-phosphogluconate dehydrogenase) | | |
| Saccharomyces cerevisiae | 149 | 150 |
| GND1 (6-phosphogluconate dehydrogenase) | | |
| Lactobacillus plantarum WCFS1 | 151 | 152 |
| gnd1 (6-phosphogluconate dehydrogenase) | | |
| Lactobacillus plantarum WCFS1 | 153 | 154 |
| gnd2 (6-phosphogluconate dehydrogenase) | | |

TABLE 4
List of SEQ ID Numbers for
Genes and Proteins of Various Reactions of the
EMP and Redox Metabolism

| | SEQ ID NO: | SEQ ID |
|---|---|---|
| | Nucleic | NO: |
| Description | acid | Peptide |
| Escherichia coli K12 MG1655 | 155 | 156 |
| pgi (glucose-6-phosphate isomerase) | | |
| Saccharomyces cerevisiae | 159 | 160 |
| PGI1 (glucose-6-phosphate isomerase) | | |
| Lactobacillus plantarum WCFS1 | 161 | 162 |
| pgi (glucose-6-phosphate isomerase) | | |
| Escherichia coli K12 MG1655 | 163 | 164 |
| pfkB (6-phosphofructokinase) | | |
| Escherichia coli K12 MG1655 | 165 | 166 |
| pfkA (6-phosphofructokinase) | | |
| Saccharomyces cerevisiae | 171 | 172 |
| PFK1 (6-phosphofructokinase) | | |
| Saccharomyces cerevisiae | 173 | 174 |
| PFK2 (6-phosphofructokinase) | | |
| Lactobacillus plantarum WCFS1 | 175 | 176 |
| pfkA (6-phosphofructokinase) | | |
| Escherichia coli K12 MG1655 | 177 | 178 |
| fbaB (fructose-bisphosphate aldolase) | | |
| Escherichia coli K12 MG1655 | 179 | 180 |
| fbaA (fructose-bisphosphate aldolase) | | |
| Saccharomyces cerevisiae | 185 | 186 |
| FBA1 (fructose-bisphosphate aldolase) | | |
| Lactobacillus plantarum WCFS1 | 187 | 188 |
| fba (fructose-bisphosphate aldolase) | | |
| Escherichia coli K12 MG1655 sthA | 257 | 258 |
| (soluble transhydrogenase) | | |

TABLE 5
List of SEQ ID Numbers of Primers

| | SEQ ID NO: |
|---|---|
| | Nucleic |
| Description | acid |
| GND H1 | 227 |
| GND H2 | 228 |
| GND Ck UP | 229 |
| GND Ck Dn | 230 |
| pCL1925 vec F | 235 |
| pCL1925 vec R1 | 236 |
| 4219-T7 | 237 |
| 4219-T8 | 238 |
| 4219-T9 | 239 |
| 4219-T10 | 240 |
| 4219-T11 | 241 |
| 4219-T12 | 242 |
| 4219-T13 | 243 |
| 4219-T14 | 244 |
| 4219-T3 | 245 |
| 4219-T4 | 246 |
| 4219-T1 | 247 |
| 4219-T2 | 248 |
| 4219-T5 | 249 |
| 4219-T6 | 225 |
| pRS411::GPM-gsdA-ADH1t vector | 226 |
| pFP996PIdhL1 vector | 142 |
| FP996-gsdA-up (primer) | 141 |
| FP996-gsdA-down (primer) | 184 |
| N473 (forward) | 231 |
| N469 (reverse) | 232 |
| N695A | 233 |
| N696A | 234 |
| pflB CkUp | 297 |
| pflB CkDn | 298 |
| frdB CkUp | 299 |
| frdB CkDn | 300 |
| ldhA CkUp | 301 |
| ldhA CkDn | 302 |
| adhE CkUp | 303 |
| adhE CkDn | 304 |
| gnd CkF | 305 |
| gnd CkR | 306 |
| pfkA CkF | 307 |
| pfkA CkR2 | 308 |
| pfkB CkF2 | 309 |
| pfkB CkR2 | 310 |
| fbaA H1 P1 lox | 311 |
| fbaA H2 P4 lox | 312 |
| fbaA Ck UP | 313 |
| fbaA Ck Dn | 314 |
| fbaB CkF2 | 315 |
| fbaB CkR2 | 316 |
| EE F | 317 |
| EE R | 318 |
| EE Seq F2 | 319 |
| EE Seq F4 | 320 |
| EE Seq R4 | 321 |
| EE Seq R3 | 322 |

TABLE 6
List of SEQ ID Numbers of Enzymes Involved
in Byproduct Formation

| | Amino | Nucleic |
|---|---|---|
| | Acid | Acid |
| | SEQ ID | SEQ ID |
| Description | NO: | NO: |
| pflB pyruvate formate lyase from E. coli | 259 | 260 |
| frdA from E. coli | 261 | 262 |
| frdB from E. coli | 263 | 264 |
| frdC from E. coli | 265 | 266 |
| frdD from E. coli | 267 | 268 |
| adhE alcohol dehydrogenase from E. coli | 269 | 270 |
| ldhA lactate dehydrogenase from E. coli | 271 | 272 |
| ldhL2 lactate dehydrogenase from L. plantarum | 273 | 274 |
| ldhD lactate dehydrogenase from L. plantarum | 275 | 276 |
| ldhL1 lactate dehydrogenase from L. plantarum | 277 | 278 |
| PDC1 pyruvate decarboxylase from | 280 | 279 |
| Saccharomyces cerevisiae | | |
| PDC5 pyruvate decarboxylase from | 282 | 281 |
| Saccharomyces cerevisiae | | |
| PDC6 pyruvate decarboxylase from | 284 | 283 |
| Saccharomyces cerevisiae | | |
| pyruvate decarboxylase from Candida | 286 | 285 |
| glabrata | | |
| PDC1 pyruvate decarboxylase from Pichia | 288 | 287 |
| stipitis | | |
| PDC2 pyruvate decarboxylase from Pichia | 290 | 289 |
| stipitis | | |
| pyruvate decarboxylase from | 292 | 291 |
| Kluyveromyces lactis | | |
| pyruvate decarboxylase from Yarrowia | 294 | 293 |
| lipolytica | | |
| pyruvate decarboxylase from | 296 | 295 |
| Schizosaccharomyces pombe | | |

The following sequences have also been used in this disclosure:
SEQ ID NO: 218 is the CUP1 promoter for Saccharomyces cerevisiae.
SEQ ID NO: 219 is the CYC1 terminator for Saccharomyces cerevisiae.
SEQ ID NO: 220 is the FBA promoter for Saccharomyces cerevisiae.
SEQ ID NO: 222 is the ADH1 terminator for Saccharomyces cerevisiae.
SEQ ID NO: 224 is the GPM promoter for Saccharomyces cerevisiae.
SEQ ID NO: 250 is the lactate dehydrogenase (IdhL1) promoter region for Lactobacillus plantarum.
SEQ ID NOs: 323-328 are genomic DNA sequences (gene coding sequence plus 1 kb upstream and 1 kb downstream) corresponding to PFK1, PFK2, FBA1, GND1, GND2, and PGI1 respectively.

### DETAILED DESCRIPTION OF THE INVENTION

Applicants have solved the problem stated above by developing a number of production hosts containing modifications to genes involved and/or influencing carbon flux through EDP, oxidative PPP and the EMP as well as associated redox metabolism.

The present invention relates to recombinant microorganisms useful for the production of isobutanol and meets a number of commercial and industrial needs. Additionally, recombinant microorganisms provided herein can be used in the production of isobutanol from plant derived carbon sources thus avoiding the negative environmental impact associated with standard petrochemical processes for butanol production.

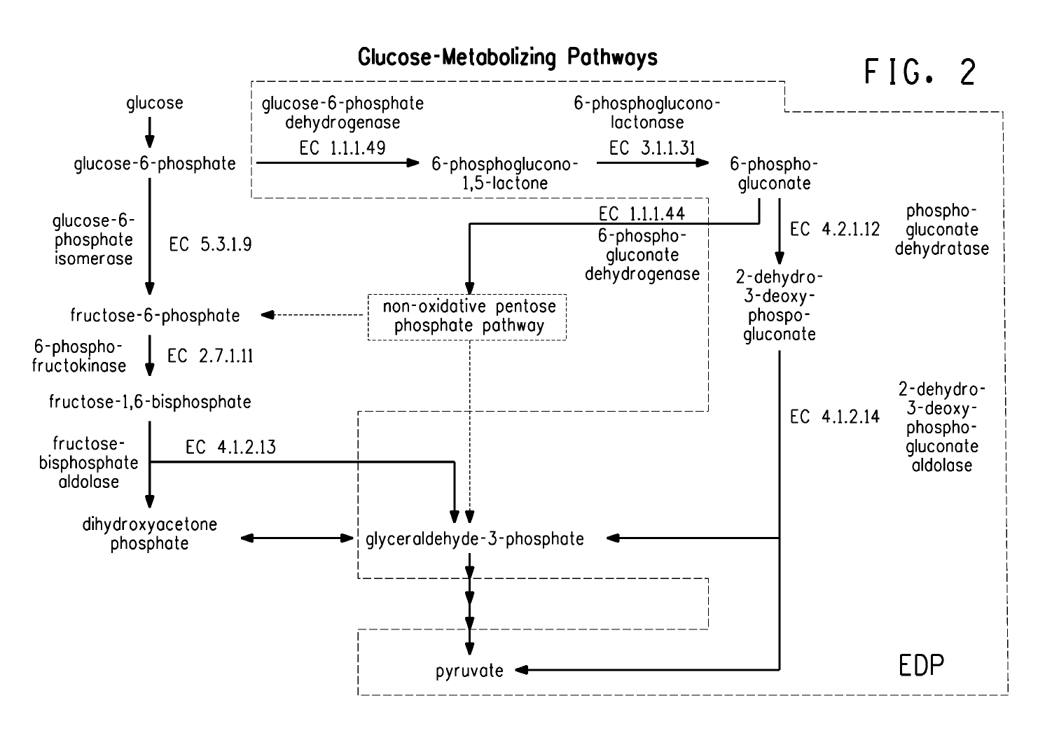

In most carbohydrate utilizing microorganisms metabolism of central metabolites, glucose- or fructose-derivatives respectively, to pyruvate occurs via at least one of the PPP, the EMP or the EDP. All of these pathways share a common intermediate, glyceraldehyde-3-phosphate, which is ultimately converted to pyruvate by a subset of EMP reactions (see FIG. 2). The combined reactions resulting in conversion of a carbon substrate to pyruvate produce energy (e.g., ATP) and reducing equivalents (e.g. NADH+H+ and NADPH+H+). NADH+H+ and NADPH+H+ must be recycled to their oxidized forms (NAD+ and NADP+, respectively) for cell growth and viability. In aerobic or permissive conditions, the inorganic electron acceptor O₂ is readily available, thus, the reducing equivalents may be used to augment the energy pool. Alternatively, in anaerobic conditions, carbon by-products may be formed, like e.g. CO₂, lactic acid, ethanol, formate, succinate, glycerol and/or others to balance the reducing equivalents.

The following definitions and abbreviations are to be used for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “invention” or “present invention” as used herein is a non-limiting term and is not intended to refer to any single embodiment of the particular invention but encompasses all possible embodiments as described in the specification and the claims.

The term “NADH” means reduced nicotinamide adenine dinucleotide.

The term “NADPH” means reduced nicotinamide adenine dinucleotide phosphate.

The term “ATP” means adenosine-5′-triphosphate. The term “H⁺” means a proton.

The terms “k_{cat}” and “Kₘ” are known to those skilled in the art and are described in Enzyme Structure and Mechanism, 2_{nd} ed. (Ferst; W.H. Freeman: NY, 1985; pp 98-120). The term “k_{cat}”, often called the “turnover number”, is defined as the maximum number of substrate molecules converted to products per active site per unit time, or the number of times the enzyme turns over per unit time. k_{cat}=Vₘₐₓ/[E], where [E] is the enzyme concentration (Ferst, supra).

The term “flux” refers to an amount of a compound that is either transported to a different location or reacted into a different compound within a certain time. For a single enzyme reaction, for example, flux is proportional to the enzyme's reaction rate. In this case, the proportionality constant is determined through the stoichiometric coefficients of the reaction, the measuring unit of the balanced compound (e.g. number of molecules, weight, number of carbon atoms, etc.) and the direction of the reaction. Typical units are “millimole per hour” (mmol/h), referring to a molar flux, “gram per hour” (g/h), referring to a weight flux, or “millimole carbon atoms per hour” (mmol(C)/h), referring to a molar carbon flux.

The term “volumetric flux” as used herein means a flux in a specified volume. Typical units are “millimole per liter per hour” (mmol/l/h), referring to a volumetric molar flux, “gram per liter per hour” (g/l/h), referring to a volumetric weight flux, or “millimole carbon atoms per hour” (mmol(C)/l/h), referring to a volumetric molar carbon flux. If the flux results exclusively from an intracellular reaction, the volumetric flux can be calculated through multiplication of the biomass concentration with the “specific flux”, as defined below.

The term “specific flux” is a flux normalized by the concentration of biomass dry weight of the biocatalyst/cell that catalyzes the reaction. Typical units are “millimole per gram dry weight per hour” (mmol/g(DW)/h) or gram per gram dry weight per hour” (g/g(DW)/h).

The term “relative flux” is the specific flux in carbon mol-units, normalized by the specific carbon-molar carbohydrate uptake rate, expressed as a percentage. If no carbon atoms are involved in a reaction, relative flux is normalized to the molar carbohydrate rate.

The term “chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

The term “expression”, as used herein, refers to the transcription and stable accumulation of sense (mRNA) or antisense RNA derived from the nucleic acid fragment(s) of the invention. Selected genes may be introduced into the host cell on a plasmid or they may be integrated into the chromosome. Expression may also refer to translation of mRNA into a polypeptide. Specific genes of an enzymatic pathway may be expressed in a cell or cellular compartment to produce the desired in the host cell. Selected genes may be introduced into the host cell on either a plasmid or they may be integrated into the chromosome with appropriate regulatory sequences. The activities of the genes and hence the level of the enzymes produced by them can be adjusted by means of either “up-regulation” or “down-regulation”, as described below.

The term “upregulation” or “upregulated” when used with regard to a specific gene or set of genes (e.g. encoding a metabolic pathway) means molecular manipulations done to a particular gene or set of genes (e.g., encoding a metabolic pathway), the process of its transcription, translation and/or the molecular properties of the involved molecules in these process, that result in increasing the amount and/or activity of the particular protein or set of proteins encoded by that gene or set of genes. For example, additional copies of selected genes may be introduced into the host cell on multicopy plasmids such as 2 micron vectors (e.g., pRS423 or pHR81), ColE1 vectors (e.g. pUC or pBR322). Such genes may also be integrated into the chromosome with appropriate regulatory sequences that result in increased amount and/or activity of their encoded functions. The genes may be modified so as to be under the control of non-native promoters or altered native promoters, yielding a chimeric gene or a set of chimeric genes. The gene sequences may also be modified in a way that secondary structure of their transcript is affected in order to prevent loops and hairpins that influence transcription efficiency or RNA stability. Endogenous promoters can be altered in vivo by mutation, deletion, and/or substitution.

The term “downregulation” or “downregulated” with reference to a specific gene or set of genes (e.g. encoding a metabolic pathway) means molecular manipulation done to a particular gene or set of genes (e.g. encoding a metabolic pathway), the process of its transcription, translation and/or the molecular properties of the involved molecules in these process, that results in decreasing the amount and/or activity of the particular protein or set of proteins encoded by that gene or set of genes. For the purposes of this invention, it is useful to distinguish between reduction and elimination. “Downregulation” and “downregulating” of a gene refers to a reduction, but not a total elimination, of the amount and/or activity of the encoded protein. Methods of downregulating genes are known to those of skill in the art. Downregulation can occur by deletion, insertion, or alteration of coding regions and/or regulatory (promoter) regions. Specific down regulations may be obtained by random mutation followed by screening or selection, or, where the gene sequence is known, by direct intervention by molecular biology methods known to those skilled in the art. A particularly useful, but not exclusive, method to achieve downregulation is to alter promoter strength.

“Deletion” or “deleted” or “disruption” or “disrupted” or “elimination” or “eliminated” used with regard to a gene or set of genes describes various activities for example, 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes). Such changes would either prevent expression of the protein of interest or result in the expression of a protein that is non-functional/shows no activity. Specific disruptions may be obtained by random mutation followed by screening or selection, or, in cases where the gene sequences are known, specific disruptions may be obtained by direct intervention using molecular biology methods know to those skilled in the art.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation, natural transduction, natural transposition) such as those occurring without deliberate human intervention. The term “Entner-Doudoroff pathway” or “EDP”, also known as “phosphorylated Entner-Doudoroff pathway” or “phosphorylated EDP”, refers to a sequence of reactions, comprising glucose-6-phosphate dehydrogenase reaction, 6-phosphogluconolactonase reaction, phosphogluconate dehydratase reaction, and 2-dehydro-3-deoxy-phosphogluconate aldolase reaction. The term “functional Entner-Doudoroff pathway” or “functional EDP” refers to the aforementioned sequence of EDP reactions, whereas every single reaction step can exhibit a relative flux of at least 1% under permissive conditions. The term “enhanced Entner-Doudoroff pathway” or “enhanced EDP” refers to the afore mentioned sequence of EDP reactions, whereas at least one reaction step has a relative flux that is at least 1% higher when compared to the relative flux of the respective reaction in a microbial host or cultivation environment without enhanced EDP. In some embodiments, the relative flux is at least 5% higher under permissive conditions, and in some embodiments, the relative flux is at least 10% higher under permissive conditions. A host cell that lacks a native EDP but that is engineered to contain a functional EDP necessarily contains an “enhanced EDP” as used herein.

The term “oxidative Pentose Phosphate Pathway” or “oxidative PPP” refers to a sequence of reactions, comprising glucose-6-phosphate dehydrogenase reaction, 6-phosphogluconolactonase reaction, and 6-phosphogluconate dehydrogenase reaction. The term “functional oxidative Pentose Phosphate Pathway” or “functional oxidative PPP” refers to the aforementioned sequence of oxidative PPP reactions, whereas every single reaction step can exhibit a relative flux of at least 1% under permissive conditions. The term “diminished oxidative Pentose Phosphate Pathway” or “diminished oxidative PPP” refers to the afore mentioned sequence of oxidative PPP reactions, whereas at least one reaction step has a relative flux that is at least 1% lower when compared to the relative flux of the respective reaction in a microbial host or cultivation environment without diminished oxidative PPP. In some embodiments, the relative flux is at least 5% lower under permissive conditions, and in some embodiments, the relative flux is at least 10% lower under permissive conditions. The term “non-oxidative Pentose Phosphate Pathway” or “non-oxidative PPP” refers to a sequence of reactions, comprising the ribose-5-phosphate isomerase reaction, the ribulose-5-phosphate 3-epimerase reaction, a transketolase and two transaldolase reactions.

The term “Pentose Phosphate Pathway” or “PPP” refers to a sequence of reactions, comprising the reactions of the oxidative as well as of the non-oxidative PPP. The term “Embden-Meyerhof Pathway”, “EMP” or “glycolysis” refers to a sequence of reactions, comprising glucokinase and/or hexokinase reaction, glucose-6-phosphate isomerase reaction, reaction, fructose-bisphosphate aldolase reaction, triose-phosphate isomerase reaction, glyceraldehyde-3-phosphate dehydrogenase reaction, 3-phosphoglycerate kinase reaction, phosphoglyceromutase reaction, enolase reaction, and pyruvate kinase reaction. The term “functional Embden-Meyerhof Pathway” or “functional EMP” refers to the aforementioned sequence of EMP reactions, whereas every single reaction step can exhibit a relative flux of at least 1% under permissive conditions. The term “diminished Embden-Meyerhof pathway”, “diminished EMP” or “diminished glycolysis” refers to the afore mentioned sequence of EMP reactions, whereas at least one reaction step has a relative flux that is at least 1% lower when compared to the relative flux of the respective reaction in a microbial host or cultivation environment without diminished EMP. In some embodiments, the relative flux is at least 5% lower under permissive conditions, and in some embodiments, the relative flux is at least 10% lower under permissive conditions.

The increase or decrease in relative flux is herein equated to the degree of enhancement or diminishment. For example, an enhanced EDP demonstrating a relative flux that is about 10% higher can be said to be enhanced by about 10%. Likewise, an EMP demonstrating a relative flux that is about 10% decreased can be said to be diminished by about 10%.

One of skill in the art will appreciate that certain of the reactions of the EDP are not common with those of the EMP or PPP, and likewise, certain of the reactions of the EMP are not common with those of the PPP. Such reactions are herein referred to as “unique” to the pathway. For example, reactions of the EDP which are not common with those of the EMP or PPP are thus referred to as “unique to the EDP” herein.

The term “isobutanol biosynthetic pathway” refers to an enzymatic pathway to produce isobutanol. Exemplary isobutanol biosynthetic pathways are discussed and described in U.S. Patent Application Publication No. 20070092957, incorporated herein by reference in its entirety.

The terms “acetolactate synthase” and “acetolactate synthetase” are used interchangeably herein to refer to an enzyme that catalyzes the conversion of pyruvate to acetolactate and CO₂. Preferred acetolactate synthases are known by the EC number 2.2.1.6 (Enzyme Nomenclature 1992, Academic Press, San Diego). These enzymes are available from a number of sources, including, but not limited to, Bacillus subtilis (GenBank No: CAB15618, amino acid SEQ ID NO:2, nucleic acid SEQ ID NO:1; NCBI (National Center for Biotechnology Information)), Klebsiella pneumoniae (GenBank No: AAA25079, amino acid SEQ ID NO:4, nucleic acid SEQ ID NO:3), and Lactococcus lactis (GenBank No: AAA25161, amino acid SEQ ID NO:6, nucleic acid SEQ ID NO:5).

The terms “acetohydroxy acid isomeroreductase” and “acetohydroxy acid reductoisomerase” are used interchangeably herein to refer to an enzyme that catalyzes the conversion of acetolactate to 2,3-dihydroxyisovalerate using NADPH (reduced nicotinamide adenine dinucleotide phosphate) as an electron donor. Preferred acetohydroxy acid isomeroreductases are known by the EC number 1.1.1.86 and sequences are available from a vast array of microorganisms, including, but not limited to, Escherichia coli (GenBank No: NP_{—}418222, amino acid SEQ ID NO:8, nucleic acid SEQ ID NO:7), Saccharomyces cerevisiae (GenBank No: NP_{—}013459, amino acid SEQ ID NO:10, nucleic acid SEQ ID NO:9), Methanococcus maripaludis (GenBank No: CAF30210, amino acid SEQ ID NO:12, nucleic acid SEQ ID NO:11), and Bacillus subtilis (GenBank No: CAB14789, amino acid SEQ ID NO:14, nucleic acid SEQ ID NO:13).

The term “acetohydroxy acid dehydratase” refers to an enzyme that catalyzes the conversion of 2,3-dihydroxyisovalerate to α-ketoisovalerate. Preferred acetohydroxy acid dehydratases are known by the EC number 4.2.1.9. These enzymes are available from a vast array of microorganisms, including, but not limited to, E. coli (GenBank No: YP_{—}026248, amino acid SEQ ID NO:16, nucleic acid SEQ ID NO:15), S. cerevisiae (GenBank No: NP_{—}012550, amino acid SEQ ID NO:18, nucleic acid SEQ ID NO:17), Methanococcus maripaludis (GenBank No: CAF29874, amino acid SEQ ID NO: 20, nucleic acid SEQ ID NO:19), and B. subtilis (GenBank No: CAB14105, amino acid SEQ ID NO:22, nucleic acid SEQ ID NO:21).

The term “branched-chain α-keto acid decarboxylase” refers to an enzyme that catalyzes the conversion of α-ketoisovalerate to isobutyraldehyde and CO₂. Preferred branched-chain α-keto acid decarboxylases are known by the EC number 4.1.1.72 and are available from a number of sources, including, but not limited to, Lactococcus lactis (GenBank No: AAS49166, amino acid SEQ ID NO:24, nucleic acid SEQ ID NO:23; CAG34226, amino acid SEQ ID NO:26, L. lactis codon optimized kivD nucleic acid SEQ ID NO: 25, nucleic acid SEQ ID NO:189), Salmonella typhimurium (GenBank No: NP_{—}461346, amino acid SEQ ID NO:28, nucleic acid SEQ ID NO:27), and Clostridium acetobutylicum (GenBank No: NP_{—}149189, amino acid SEQ ID NO:30, nucleic acid SEQ ID NO:29).

The term “branched-chain alcohol dehydrogenase” refers to an enzyme that catalyzes the conversion of isobutyraldehyde to isobutanol. Preferred branched-chain alcohol dehydrogenases are known by the EC number 1.1.1.265, but may also be classified under other alcohol dehydrogenases (specifically, EC 1.1.1.1 or 1.1.1.2). These enzymes preferably utilize NADH (reduced nicotinamide adenine dinucleotide) and/or, less preferably, NADPH as electron donor and are available from a number of sources, including, but not limited to, S. cerevisiae (GenBank No: NP_{—}010656, amino acid SEQ ID NO:32, nucleic acid SEQ ID NO:31; NP_{—}014051, amino acid SEQ ID NO:34, nucleic acid SEQ ID NO:33), E. coli (GenBank No: NP_{—}417-484, amino acid SEQ ID NO:36, nucleic acid SEQ ID NO: 35), and C. acetobutylicum (GenBank No: NP_{—}349892, amino acid SEQ ID NO: 38, nucleotide SEQ ID NO:37; NP_{—}349891, amino acid SEQ ID NO:40, nucleic acid SEQ ID NO:39).

The term “branched-chain keto acid dehydrogenase” refers to an enzyme that catalyzes the conversion of α-ketoisovalerate to isobutyryl-CoA (isobutyryl-coenzyme A), using NAD⁺ (nicotinamide adenine dinucleotide) as electron acceptor. Preferred branched-chain keto acid dehydrogenases are known by the EC number 1.2.4.4. These branched-chain keto acid dehydrogenases are comprised of four subunits and sequences from all subunits are available from a vast array of microorganisms, including, but not limited to, B. subtilis (GenBank No: CAB14336, amino acid SEQ ID NO:42, nucleic acid SEQ ID NO:41; CAB14335, amino acid SEQ ID NO:44, nucleic acid SEQ ID NO:43; CAB14334, amino acid SEQ ID NO:46, nucleic acid SEQ ID NO:45; and CAB14337, amino acid SEQ ID NO:48, nucleic acid SEQ ID NO:47) and Pseudomonas putida (GenBank No: AAA65614, amino acid SEQ ID NO:50, nucleic acid SEQ ID NO:49; AAA65615, amino acid SEQ ID NO:52, nucleic acid SEQ ID NO:51; AAA65617, amino acid SEQ ID NO:54, nucleic acid SEQ ID NO:53; and AAA65618, amino acid SEQ ID NO:56, nucleic acid SEQ ID NO:55).

The term “acylating aldehyde dehydrogenase” refers to an enzyme that catalyzes the conversion of isobutyryl-CoA to isobutyraldehyde, using either NADH or NADPH as electron donor. Preferred acylating aldehyde dehydrogenases are known by the EC numbers 1.2.1.10 and 1.2.1.57. These enzymes are available from multiple sources, including, but not limited to, Clostridium beijerinckii (GenBank No: AAD31841, amino acid SEQ ID NO:58, nucleic acid SEQ ID NO:57), C. acetobutylicum (GenBank No: NP_{—}149325, amino acid SEQ ID NO:60, nucleic acid SEQ ID NO:59; NP_{—}149199, amino acid SEQ ID NO:62, nucleic acid SEQ ID NO:61), P. putida (GenBank No: AAA89106, amino acid SEQ ID NO:64, nucleic acid SEQ ID NO:63), and Thermus thermophilus (GenBank No: YP_{—}145486, amino acid SEQ ID NO:66, nucleic acid SEQ ID NO:65).

The term “transaminase” refers to an enzyme that catalyzes the conversion of α-ketoisovalerate to L-valine, using either alanine or glutamate as amine donor. Preferred transaminases are known by the EC numbers 2.6.1.42 and 2.6.1.66. These enzymes are available from a number of sources. Examples of sources for alanine-dependent enzymes include, but are not limited to, E. coli (GenBank No: YP_{—}026231, amino acid SEQ ID NO:68, nucleic acid SEQ ID NO:67) and Bacillus licheniformis (GenBank No: YP_{—}093743, amino acid SEQ ID NO:70, nucleic acid SEQ ID NO:69). Examples of sources for glutamate-dependent enzymes include, but are not limited to, E. coli (GenBank No: YP_{—}026247, amino acid SEQ ID NO:72, nucleic acid SEQ ID NO:71), S. cerevisiae (GenBank No: NP_{—}012682, amino acid SEQ ID NO:74, nucleic acid SEQ ID NO:73) and Methanobacterium thermoautotrophicum (GenBank No: NP_{—}276546, amino acid SEQ ID NO:76, nucleic acid SEQ ID NO:75).

The term “valine dehydrogenase” refers to an enzyme that catalyzes the conversion of α-ketoisovalerate to L-valine, using NAD(P)H as electron donor and ammonia as amine donor. Preferred valine dehydrogenases are known by the EC numbers 1.4.1.8 and 1.4.1.9 and are available from a number of sources, including, but not limited to, Streptomyces coelicolor (GenBank No: NP_{—}628270, amino acid SEQ ID NO:78, nucleic acid SEQ ID NO:77) and B. subtilis (GenBank Nos: CAB14339, amino acid SEQ ID NO:80, nucleic acid SEQ ID NO:79).

The term “valine decarboxylase” refers to an enzyme that catalyzes the conversion of L-valine to isobutylamine and CO₂. Preferred valine decarboxylases are known by the EC number 4.1.1.14. These enzymes are found in Streptomycetes, such as for example, Streptomyces viridifaciens (GenBank No: AAN10242, amino acid SEQ ID NO:82, nucleic acid SEQ ID NO:81).

The term “omega transaminase” refers to an enzyme that catalyzes the conversion of isobutylamine to isobutyraldehyde using a suitable amino acid as amine donor. Preferred omega transaminases are known by the EC number 2.6.1.18 and are available from a number of sources, including, but not limited to, Alcaligenes denitrificans (AAP92672, amino acid SEQ ID NO:84, nucleic acid SEQ ID NO:83), Ralstonia eutropha (GenBank No: YP_{—}294474, amino acid SEQ ID NO:86, nucleic acid SEQ ID NO:85), Shewanella oneidensis (GenBank No: NP_{—}719046, amino acid SEQ ID NO:88, nucleic acid SEQ ID NO:87), and P. putida (GenBank No: AAN66223, amino acid SEQ ID NO:90, nucleic acid SEQ ID NO:89).

The term “isobutyryl-CoA mutase” refers to an enzyme that catalyzes the conversion of butyryl-CoA to isobutyryl-CoA. This enzyme uses coenzyme B₁₂ as cofactor. Preferred isobutyryl-CoA mutases are known by the EC number 5.4.99.13. These enzymes are found in a number of Streptomycetes, including, but not limited to, Streptomyces cinnamonensis (GenBank Nos: AAC08713, amino acid SEQ ID NO:92, nucleic acid SEQ ID NO:91; CAB59633, amino acid SEQ ID NO:94, nucleic acid SEQ ID NO:93), S. coelicolor (GenBank No: CAB70645, amino acid SEQ ID NO:96, nucleic acid SEQ ID NO:95; CAB92663, amino acid SEQ ID NO:98, nucleic acid SEQ ID NO:97), and Streptomyces avermitilis (GenBank No: NP_{—}824008, amino acid SEQ ID NO:100, nucleic acid SEQ ID NO:99); NP_{—}824637, amino acid SEQ ID NO:102, nucleic acid SEQ ID NO:101).

The term “glucose-6-phosphate dehydrogenase”, also known as “6-phosphoglucose dehydrogenase”, “D-glucose 6-phosphate dehydrogenase”, “gdpd”, “G6PDH”, “NADP-dependent glucose 6-phosphate dehydrogenase” or “NADP-glucose-6-phosphate dehydrogenase”, refers to an enzyme that catalyzes the conversion of glucose-6-phosphate to 6-phosphogluconolactone, using either NAD⁺ or NADP⁺ as electron acceptor. Preferred glucose-6-phosphate dehydrogenases are known by the EC number 1.1.1.49. These enzymes are available from a number of sources, including, but not limited to, Aspergillus niger (GenBank No: CAA61194.1, DNA SEQ ID NO: 117, Protein SEQ ID NO: 118), Aspergillus nidulans (GenBank No: XP_{—}660585.1, DNA SEQ ID NO: 119, Protein SEQ ID NO:120), Schizosaccharomyces pombe (GenBank Nos: NP_{—}587749.1, DNA SEQ ID NO: 123, Protein SEQ ID NO:122, and NP_{—}593614.1, DNA SEQ ID NO: 124, Protein SEQ ID NO:125, and NP_{—}593344.2, DNA SEQ ID NO: 121, Protein SEQ ID NO:126), Escherichia coli (E. coli K12 MG1655, GenBank Nos: NP 416366.1, DNA SEQ ID NO: 127, Protein SEQ ID NO:128), Lactobacillus plantarum (GenBank No: NP_{—}786078.1, DNA SEQ ID NO: 131, Protein SEQ ID NO:132)) and Saccharomyces cerevisiae (GenBank No: NP_{—}014158.1, DNA SEQ ID NO: 133, Protein SEQ ID NO:134).

The term “6-phosphogluconolactonase”, also known as “6-PGL” or “6-phospho-D-glucose-delta-lactone hydrolase”, refers to an enzyme that catalyzes the conversion of 6-phosphogluconolactone to 6-phosphogluconate. Preferred 6-phosphogluconolactonases are known by the EC number 3.1.1.31. These enzymes are available from a number of sources, including, but not limited to Escherichia coli (E. coli K12 MG1655, GenBank Nos: NP_{—}415288.1, DNA SEQ ID NO: 105, Protein SEQ ID NO:106), Lactobacillus plantarum (GenBank No: NP_{—}785709.1, DNA SEQ ID NO: 111, Protein SEQ ID NO:112), Saccharomyces cerevisiae (GenBank No: NP_{—}011764.1, DNA SEQ ID NO: 107, Protein SEQ ID NO:108) and (GenBank No: NP_{—}012033 DNA SEQ ID NO: 190, Protein SEQ ID NO:191)) and Zymomonas mobilis (GenBank No: YP_{—}163213.1, DNA SEQ ID NO: 113, Protein SEQ ID NO:114) and EBI_Protein-ID AAV90102.1, DNA SEQ ID NO: 113, Protein SEQ ID NO:114)).

The term “phosphogluconate dehydratase”, also known as “6-phospho-D-gluconate hydrolyase”, “6-PG dehydrase” or “gluconate 6-phosphate dehydratase”, refers to an enzyme that catalyzes the conversion of 6-phospho-gluconate to 2-dehydro-3-deoxy-6-phosphogluconate. Preferred phospho-gluconate dehydratases are known by the EC number 4.2.1.12. These enzymes are available from a number of sources, including, but not limited to Zymomonas mobilis (GenBank No: YP_{—}162103.1, DNA SEQ ID NO: 135, Protein SEQ ID NO:136), Pseudomonas putida (GenBank No: NP_{—}743171.1, DNA SEQ ID NO: 137, Protein SEQ ID NO:138) and Escherichia coli (E. coli K12 MG1655, GenBank Nos: NP_{—}416365.1, DNA SEQ ID NO: 139, Protein SEQ ID NO:140).

The term “2-dehydro-3-deoxy-phosphogluconate aldolase”, also known as “2-Keto-3-deoxy-6-phosphogluconate aldolase”, “2-Oxo-3-deoxy-6-phosphogluconate aldolase”, “6-phospho-2-dehydro-3-deoxy-D-gluconate D-glyceraldehyde-3-phosphate-lyase”, “6-Phospho-2-keto-3-deoxygluconate aldolase”, “Phospho-2-keto-3-deoxygluconic aldolase”, “KDGA”, “KDPG” or “KDPG aldolase”, refers to an enzyme that catalyzes the conversion of 2-dehydro-3-deoxy-6-phosphogluconate to pyruvate and glyceraldehyde 3-phosphate. Preferred 2-dehydro-3-deoxy-phosphogluconate aldolases are known by the EC number 4.1.2.14. These enzymes are available from a number of sources, including, but not limited to Azotobacter vinelandii (GenBank Nos: ZP_{—}00417447.1, DNA SEQ ID NO: 194, Protein SEQ ID NO: 195, and ZP_{—}00415409.1, DNA SEQ ID NO: 196, Protein SEQ ID NO: 197, and ZP_{—}00416840.1, DNA SEQ ID NO: 198, Protein SEQ ID NO: 199, and ZP_{—}00419301.1, DNA SEQ ID NO: 200, Protein SEQ ID NO: 201), Pseudomonas putida (GenBank No: NP_{—}743185.1, DNA SEQ ID NO: 202, Protein SEQ ID NO: 203), Pseudomonas fluorescens (GenBank No: YP_{—}261692.1, DNA SEQ ID NO: 204, Protein SEQ ID NO: 205), Zymomonas mobilis (GenBank No: YP_{—}162732.1, DNA SEQ ID NO: 206, Protein SEQ ID NO: 207) and Escherichia coli (E. coli K12 MG1655, GenBank Nos: NP_{—}416364.1, DNA SEQ ID NO: 208, Protein SEQ ID NO: 209).

The term “glucose-6-phosphate isomerase”, also known as “D-glucose-6-phosphate aldose-ketose-isomerase”, “D-glucose-6-phosphate isomerase”, “hexosephosphate isomerase”, “PGI”, “phosphoglucoisomerase”, “phosphoglucose isomerase”, “phosphohexoisomerase”, “phosphohexomutase”, “phosphohexose isomerase”, refers to an enzyme that catalyzes the conversion of glucose 6-phosphate to fructose 6-phosphate. Preferred glucose-6-phosphate isomerases are known by the EC number 5.3.1.9. These enzymes are known to occur in, but not be limited to, Escherichia coli (E. coli K12 MG1655, GenBank: GeneID:948535, DNA SEQ ID NO: 155, Protein SEQ ID NO:156), Saccharomyces cerevisiae (GenBank: GeneID:852495, DNA SEQ ID NO: 159, Protein SEQ ID NO:160) and Lactobacillus plantarum (GenBank: GeneID:1062659, DNA SEQ ID NO: 161, Protein SEQ ID NO:162).

The term “6-phosphofructokinase”, also known as “ATP:D-fructose-6-phosphate 1-phosphotransferase”, “6-phosphofructose 1-kinase”, “D-fructose-6-phosphate 1-phosphotransferase”, “PFK, phospho-1,6-fructokinase” or “phosphofructokinase”, refers to an enzyme that catalyzes the conversion of fructose 6-phosphate and ATP to fructose-1,6-bisphosphate and ADP. Preferred phosphofructokinases are known by the EC number 2.7.1.11. These enzymes are known to occur in, but not be limited to, Escherichia coli (E. coli K12 MG1655, GenBank: GeneID:946230, DNA SEQ ID NO: 163, Protein SEQ ID NO:164 and GeneID:948412, DNA SEQ ID NO: 165, Protein SEQ ID NO:166), (as well as Saccharomyces cerevisiae (GenBank: GeneID:853155, DNA SEQ ID NO: 171, Protein SEQ ID NO:172, and GeneID:855245, DNA SEQ ID NO: 173, Protein SEQ ID NO:174, representing the alpha- and beta-subunit of a heterooctamer) and Lactobacillus plantarum (GenBank: GeneID:1064199, DNA SEQ ID NO: 175, Protein SEQ ID NO:176).

The term “fructose-bisphosphate aldolase”, also known as “D-fructose-1,6-bisphosphate D-glyceraldehyde-3-phosphate-lyase”, “diphosphofructose aldolase”, “FBP aldolase, fructoaldolase”, “fructose diphosphate aldolase”, “fructose-1,6-bisphosphate aldolase”, “fructose-1,6-bisphosphate triosephosphate-lyase” or “phosphofructoaldolase”, refers to an enzyme that catalyzes the conversion of fructose-1,6-bisphosphate to glycerone phosphate, also known as dihydroxyacetone phosphate, and glyceraldehyde-3-phosphate. Preferred fructose-bisphosphate aldolases are known by the EC number 4.1.2.13. These enzymes are known to occur in, but not be limited to, Escherichia coli (E. coli K12 MG1655, GenBank: GeneID:946632, DNA SEQ ID NO: 177, Protein SEQ ID NO:178) and GeneID:947415, DNA SEQ ID NO: 179, Protein SEQ ID NO:180), as well as Saccharomyces cerevisiae (GenBank: GeneID:853805, DNA SEQ ID NO: 185, Protein SEQ ID NO: 186) and Lactobacillus plantarum (GenBank: GeneID:1062165, DNA SEQ ID NO: 187, Protein SEQ ID NO: 188).

The term “6-phosphogluconate dehydrogenase”, also known as “phosphogluconate dehydrogenase (decarboxylating)”, also known as “6-phospho-D-gluconate:NADP+2-oxidoreductase (decarboxylating)”, “6-P-gluconate dehydrogenase”, “6-phospho-D-gluconate-NAD(P)+oxidoreductase”, “6-phosphogluconic dehydrogenase”, “6PGD”, “D-gluconate-6-phosphate dehydrogenase” or “phosphogluconic acid dehydrogenase” refers to an enzyme that catalyzes the conversion of 6-phosphogluconate to ribulose-5-phosphate and carbon dioxide, using either NAD⁺ or NADP⁺ as electron acceptor. Preferred 6-phosphogluconate dehydrogenases are known by the EC number 1.1.1.44. These enzymes are known to occur in, but not be limited to, Escherichia coli (E. coli K12 MG1655, GenBank: GeneID:946554 (DNA SEQ ID NO: 143, Protein SEQ ID NO:144) and, Saccharomyces cerevisiae (GenBank: GeneID:853172, DNA SEQ ID NO: 147, Protein SEQ ID NO:148) and GeneID:856589, DNA SEQ ID NO: 149, Protein SEQ ID NO:150) and Lactobacillus plantarum (GenBank: GeneID:1062968, DNA SEQ ID NO: 151, Protein SEQ ID NO:152) and GeneID:1062157, DNA SEQ ID NO: 153, Protein SEQ ID NO:154).

The term “soluble transhydrogenase”, also known as “NAD(P)⁺ transhydrogenase (B-specific)”, “NADPH:NAD⁺ oxidoreductase (B-specific)”, “NAD transhydrogenase”, “NAD(P) transhydrogenase”, “NADPH-NAD oxidoreductase”, “NADPH-NAD transhydrogenase”, “nicotinamide nucleotide transhydrogenase”, “non-energy-linked transhydrogenase”, “pyridine nucleotide transhydrogenase” or “STH”, refers to an enzyme that catalyzes the conversion of NADPH+H⁺ and NAD⁺ to NADP⁺ and NADH+H⁺. Preferred soluble transhydrogenases are known by the EC number 1.6.1.1. These enzymes are known to occur in, but not be limited to, Escherichia coli (E. coli K12 MG1655, GenBank: GeneID:948461, DNA SEQ ID NO: 257, Protein SEQ ID NO: 258.

### Optimization of Isobutanol Production

Certain isobutanol production pathways useful in production organisms have a specific co-factor requirement of one NADH and one NADPH for every 2 molecules of pyruvate processed to isobutanol. While not wishing to be bound by theory, it is believed that balancing the specific cofactor requirements of an isobutanol production pathway with the reducing equivalents produced in the conversion of a substrate to pyruvate will improve production. Therefore, one embodiment provided herein is a recombinant microbial host cell comprising an alteration in the EDP, EMP, and/or PPP such that the reducing equivalents generated by the conversion of a substrate to pyruvate are matched to those cofactors required for the production of isobutanol from pyruvate. Preferred embodiments provided herein optimize isobutanol production through preferential use of a functional and/or enhanced EDP which produces one NADH and one NADPH and 2 molecules of pyruvate for each molecule of a hexose-derivative processed. Such balance may increase yield of isobutanol. Preferred yields are about 60% or greater of theoretical, with about 75% or greater of theoretical preferred, about 85% or greater of theoretical more preferred, about 90% or greater of theoretical even more preferred, and with about 95% or greater of theoretical most preferred. In some embodiments, with glucose as the substrate, isobutanol yields are greater than or equal to about 0.3 g/g, greater than or equal to about 0.33 g/g, greater than or equal to about 0.35 g/g, or greater than or equal to about 0.39 g/g.

Of the preferred hosts disclosed herein, only E. coli is currently known to have genes for the functional operation of the three pathways EMP, oxidative PPP and EDP. S. cerevisiae and L. plantarum do not have endogenous genes required for a functional EDP in their genome. In both species, no genes encoding a phosphogluconate dehydratase reaction (EC number 4.2.1.12) and a 2-dehydro-3-deoxy-phosphogluconate aldolase reaction (EC number 4.1.2.14) were identified to date.

In cases where the required components for a functional EDP are not endogenous to the host, missing enzymes can be expressed. Host cells modified in this way contain a “functional heterologous EDP”. Thus, regardless of the host cell, the relative EDP flux may be increased by introducing and/or up-regulating the respective pathway genes using recombinant DNA technology methodologies.

Examples of enzymes suitable to augment EDP pathways include the following: Glucose-6-phosphate dehydrogenases (EC-Number 1.1.1.49) of both Aspergillus niger and Aspergillus nidulans exhibit strict specificity towards both substrates glucose 6-phosphate and NADP⁺ (Wennekes, L. M., and Goosen, T., J. Gen. microbiol., 139: 2793-2800, 1993). In both Aspergilli species the glucose-6-phosphate dehydrogenase activity is regulated by the NADPH:NADP⁺ ratio. The kinetic parameters for the A. niger enzyme are: Kₘ(G6P)=153±10 μM, Kₘ(NADP⁺)=26±8 μM, vₘₐₓ=790 μmol(NADPH/min/mg(protein), while these paramters for A. nidulans enzyme are: Kₘ(G6P)=92±10 μM, Kₘ(NADP⁺)=30±8 μM, Kᵢ(NADPH)=20±5 μM, vₘₐₓ=745 μmol(NADPH/min/mg(protein).

### The Embden-Meyerhof Pathway (EMP)

The typical EMP from glucose to pyruvate comprises a sequence of 10 reactions (see FIG. 2):
(1) the hexokinase and/or glucokinase reaction, converting glucose to glucose-6-phosphate,
(2) the glucose-6-phosphate isomerase reaction, converting glucose-6-phosphate into fructose-6-phosphate,
(3) the 6-phosphofructokinase reaction, converting fructose-6-phosphate to fructose-1,6-bisphosphate,
(4) the fructose-bisphosphate aldolase reaction, converting fructose-1,6-bisphosphate to glyceraldehyde-3-phosphate and dihydroxy-acetonephosphate,
(5) the triose-phosphate isomerase reaction, converting dihydroxyacetone-phosphate to glyceraldehyde-3-phosphate
(6) the glyceraldehyde-3-phosphate dehydrogenase reaction, converting glyceraldehyde-3-phosphate to 1,3-biphosphoglycerate,
(7) the 3-phosphoglycerate kinase reaction, converting 1,3-biphosphoglycerate to 3-phosphoglycerate,
(8) the phosphoglyceromutase reaction, converting 3-phosphoglycerate into 2-phosphoglycerate,
(9) the enolase reaction, converting 2-phosphoglycerate to phosphoenolpyruvate,
(10) the pyruvate kinase reaction, converting phosphoenolpyruvate to pyruvate.

In this set of reactions only the glyceraldehyde-3-phosphate dehydrogenase reaction produces redox equivalents 2[H], typically through the generation of NADH from NAD⁺. Whereas the 6-phosphofructokinase reaction requires a phosphate group and a driving force, typically provided by the concomitant conversion of ATP to ADP and Pᵢ, the carbon compound conversions of the 3-phosphoglycerate kinase reaction and the pyruvate kinase reaction each are exergonic under most physiological conditions. The metabolic system typically salvages these energies through coupling the carbon compound conversion with the production of ATP from ADP and Pᵢ. Conversion of glucose to pyruvate using the EMP reactions (not considering the balancing of protons and electric charges) can be summarized as:

1 glucose+2 ADP+2 Pᵢ->2 pyruvate+2 ATP+4 [H]

Assuming cofactor specificity of NAD⁺ for the glyceraldehyde-3-phosphate dehydrogenase reaction, conversion of glucose to pyruvate using the PPP reactions can be summarized as:

1 glucose+2 ADP+2 Pᵢ+2 NAD⁺->2 pyruvate+2 ATP+2 NADH+H⁺

### The Pentose Phosphate Pathway (PPP)

A typical pathway from glucose to pyruvate through the PPP, comprising reactions of the oxidative and non-oxidative PPP as well as some EMP reactions consists of a sequence of 15 reactions (see FIG. 2):
(1) the hexokinase and/or glucokinase reaction, converting glucose to glucose-6-phosphate,
(2) the glucose-6-phosphate dehydrogenase reaction, converting glucose-6-phosphate to 6-phosphoglucono-1,5-lactone,
(3) the 6-phosphogluconolactonase reaction, converting 6-phosphoglucono-1,5-lactone to 6-phosphogluconate,
(4) the 6-phosphogluconate dehydrogenase reaction, converting 6-phosphogluconate to ribulose-5-phosphate and carbon dioxide,
(5) the ribose-5-phosphate isomerase reaction, converting ribulose-5-phosphate to ribose-5-phosphate,
(6) the ribulose-5-phosphate 3-epimerase reaction, converting ribulose-5-phosphate to xylulose-5-phosphate,
(7) a transketolase reaction, converting xylulose-5-phosphate and ribose-5-phosphate to sedoheptulose-7-phosphate and glyceraldehyde-3-phosphate,
(8) the transaldolase reaction, converting sedoheptulose-7-phosphate and glyceraldehyde-3-phosphate to fructose-6-phosphate and erythrose-4-phosphate,
(9) a transketolase reaction, converting erythrose-4-phosphate and xylulose-5-phosphate into fructose-6-phosphate and glyceraldehyde-3-phosphate,
(10) the glucose-6-phosphate isomerase reaction, converting fructose-6-phosphate into glucose-6-phosphate,
(11) the glyceraldehyde-3-phosphate dehydrogenase reaction, converting glyceraldehyde-3-phosphate into 1,3-biphosphoglycerate,
(12) the 3-phosphoglycerate kinase reaction, converting 1,3-biphosphoglycerate into 3-phosphoglycerate
(13) the phosphoglyceromutase reaction, converting 3-phosphoglycerate into 2-phosphoglycerate,
(14) the enolase reaction, converting 2-phosphoglycerate into phosphoenolpyruvate,
(15) the pyruvate kinase reaction, converting phosphoenolpyruvate into pyruvate.

In this pathway, the glucose-6-phosphate dehydrogenase reaction, 6-phosphogluconate dehydrogenase reaction and glyceraldehyde-3-phosphate dehydrogenase reaction produce redox equivalents, 2[H], typically through the generation of either NADPH or NADH from NADP⁺ or NAD⁺. As indicated above, the 3-phosphoglycerate kinase and the pyruvate kinase reactions are exergonic under most physiological conditions. The metabolic system typically salvages these energies through coupling the reaction with production of ATP from ADP and P. In summary, conversion of glucose to pyruvate using the PPP reactions (not considering the balancing of protons and electric charges) can be summarized as:

1 glucose+1 ADP+1 Pᵢ->1 pyruvate+3 CO₂+1 ATP+14 [H]

Assuming cofactor specificity of NADP⁺ for the glucose-6-phosphate dehydrogenase and 6-phosphogluconate dehydrogenase reactions, and NAD⁺ cofactor specificity for the glyceraldehyde-3-phosphate dehydrogenase reaction, conversion of glucose to pyruvate via PPP can be summarized as:

1 glucose+1 ADP+1 Pᵢ+6 NADP⁺+1 NAD⁺--->1 pyruvate+3 CO₂+1 ATP+6 NADPH+H⁺+1 NADH+H⁺

### The Entner-Doudoroff Pathway (EDP)

A typical pathway from glucose to pyruvate through the EDP comprises a sequence of 10 reactions (see FIG. 2):
(1) the hexokinase and/or glucokinase reaction, converting glucose to glucose-6-phosphate,
(2) the glucose-6-phosphate dehydrogenase reaction, converting glucose-6-phosphate to 6-phosphoglucono-1,5-lactone,
(3) the 6-phosphogluconolactonase reaction, converting 6-phosphoglucono-1,5-lactone to 6-phosphogluconate,
(4) the phosphogluconate dehydratase reaction, converting 6-phosphogluconate to 2-dehydro-3-deoxy-phosphogluconate,
(5) the 2-dehydro-3-deoxy-phosphogluconate aldolase reaction, converting 2-dehydro-3-deoxy-phosphogluconate to pyruvate and glyceraldehyde-3-phosphate,
(6) the glyceraldehyde-3-phosphate dehydrogenase reaction, converting glyceraldehyde-3-phosphate to 1,3-biphosphoglycerate,
(7) the 3-phosphoglycerate kinase reaction, converting 1,3-biphosphoglycerate to 3-phosphoglycerate,
(8) the phosphoglyceromutase reaction, converting 3-phosphoglycerate into 2-phosphoglycerate,
(9) the enolase reaction, converting 2-phosphoglycerate to phosphoenolpyruvate,
(10) the pyruvate kinase reaction, converting phosphoenolpyruvate to pyruvate.

In this pathway, the glucose-6-phosphate dehydrogenase reaction and glyceraldehyde-3-phosphate dehydrogenase reaction produce redox equivalents 2[H], typically through the generation of either NADPH or NADH from NADP⁺ or NAD⁺, respectively. The 3-phosphoglycerate kinase reaction and the pyruvate kinase reaction each are exergonic under most physiological conditions. The metabolic system typically salvages these energies through coupling the carbon compound conversion with the production of ATP from ADP and P. Conversion of glucose to pyruvate via EDP can be summarized as:

1 glucose+1 ADP+1 Pᵢ--->2 pyruvate+1 ATP+4 [H] Assuming cofactor specificity of NADP⁺ for the glucose-6-phosphate dehydrogenase and NAD⁺ for the glyceraldehyde-3-phosphate dehydrogenase reaction, conversion of glucose to pyruvate via the EDP can be summarized as:

1 glucose+1 ADP+1 Pᵢ--->2 pyruvate+1 ATP+1 NADH+H⁺+1 NADPH+H⁺

There are two major variants of the EDP, known as “partially phosphorylated EDP” and “non-phosphorylated EDP” (Romano, A. H., and Conway, T., Res. Microbiol., 147: 448-455, 1996). In the non-phosphorylated EDP, glucose is oxidized to gluconate by NAD(P)⁺-dependent glucose dehydrogenase (EC 1.1.1.47) and either gluconolactonase (EC 3.1.1.17) or spontaneous hydrolysis, and subsequently dehydrated by gluconate dehydratase (EC 4.2.1.39) to yield 2-dehydro-3-deoxy-6-gluconate, which is then cleaved by 2-dehydro-3-deoxy-6-gluconate aldolase to pyruvate and glyceraldehyde (Kim, S, and Lee, S. B., Biochem. J., 387: (pt 1): 271-280, 2005). This pathway was found active in S. solfataricus (De Rosa, M., and Gambacorta, A., Biochem. J., 224: 407-414, 1984), and also in the thermoacidophilic archaeon Thermoplasma acidophilum (Budgen. N., and Danson, M. J., FEBS Letters, 196: 207-210, 1986). Glyceraldehyde formed through the non-phosphorylated route is converted by glyceraldehyde dehydrogenase into glycerate, which is then phosphorylated to form 2-phosphoglycerate. This intermediate is then converted to generate one molecule of pyruvate by enolase reaction and pyruvate kinase reaction. Whereas the redox and carbon balance of the non-phosphoylated EDP is comparable with the phosphorylated EDP, the energy yield is less favorable. Assuming cofactor specificity of NADP⁺ for the glucose dehydrogenase and NAD⁺ for the glyceraldehyde dehydrogenase reaction, conversion of glucose to pyruvate via non-phosphorylated EDP (not considering the balancing of protons and electric charges) is summarized as:

1 glucose---->2 pyruvate+1 NADH+H⁺+1 NADPH+H⁺

Consequently the production of isobutanol from glucose through the non-phosphorylated pathway would not result in any net energy production, e.g. in terms of ATP formation.

The partially phosphorylated EDP was first observed in Rhodobacter sphaeroides (Szymona, M., and Doudoroff, M., J. Gen. Microbiol., 22: 167-183, 1960), and was later found in other bacteria and halophilic archaea (Conway, T., FEMS Microbiol Rev., 9: 1-27, 1992). In the partially phosphorylated EDP, glucose is converted into gluconate and 2-dehydro-3-deoxy-6-gluconate as in the non-phosphorylated EDP pathway, but the 2-dehydro-3-deoxy-6-gluconate produced by gluconate dehydratase is then phosphorylated by 2-dehydro-3-deoxy-6-gluconate kinase (EC 2.7.1.45) to 2-dehydro-3-deoxy-6-phospho-gluconate. 2-dehydro-3-deoxy-6-phospho-gluconate is then cleaved by 2-dehydro-3-deoxy-6-phosphogluconate aldolase to pyruvate and glyceraldehyde-3-phosphate and processed further in the reaction sequence already described for the discussion of the phosphorylated EDP. Assuming cofactor specificity of NADP⁺ for the glucose dehydrogenase and NAD⁺ for the glyceraldehyde-3-phosphate dehydrogenase reaction, conversion of glucose to pyruvate through the reaction sequence of the partially phosphorylated EDP (not considering the balancing of protons and electric charges) is summarized as:

1 glucose+1 ADP+1 Pᵢ--->2 pyruvate+1 ATP+1 NADH+H⁺+1 NADPH+H⁺

Consequently with respect to the overall balance for the metabolism of glucose to isobutanol and assuming the stated cofactor dependencies, there is no difference between the phosphorylated and the partially phosphorylated EDP.

### Isobutanol Biosynthetic Pathways

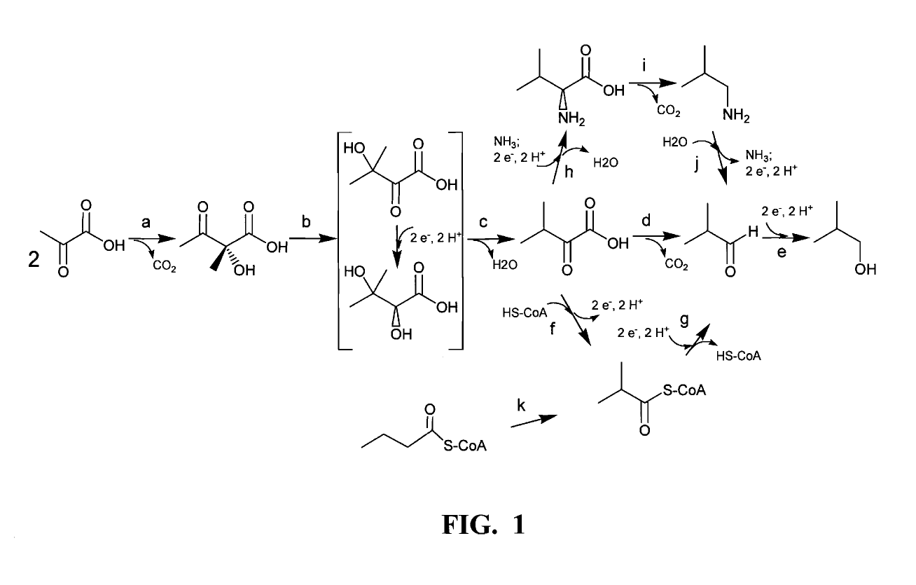

Isobutanol can be produced from carbohydrate sources with recombinant microorganisms by through various biosynthetic pathways. Preferred pathways converting pyruvate to isobutanol include the four complete reaction pathways shown in FIG. 1. A suitable isobutanol pathway (FIG. 1, steps a to e), comprises the following substrate to product conversions:
a) pyruvate to acetolactate, as catalyzed for example by acetolactate synthase,
b) acetolactate to 2,3-dihydroxyisovalerate, as catalyzed for example by acetohydroxy acid isomeroreductase,
c) 2,3-dihydroxyisovalerate to α-ketoisovalerate, as catalyzed for example by acetohydroxy acid dehydratase,
d) α-ketoisovalerate to isobutyraldehyde, as catalyzed for example by a branched-chain keto acid decarboxylase, and
e) isobutyraldehyde to isobutanol, as catalyzed for example by, a branched-chain alcohol dehydrogenase.

This pathway combines enzymes involved in pathways for valine biosynthesis (pyruvate to α-ketoisovalerate) and valine catabolism α-ketoisovalerate to isobutanol). Since many valine biosynthetic enzymes also catalyze analogous reactions in the isoleucine biosynthetic pathway, substrate specificity is a major consideration in selecting the gene sources. For this reason, the preferred genes for the acetolactate synthase enzyme are those from Bacillus (alsS) and Klebsiella (budB). These particular acetolactate synthases participate in butanediol fermentation in these organisms and show increased affinity for pyruvate over ketobutyrate (Gollop et al., J. Bacteriol. 172: 3444-3449, 1990); Holtzclaw et al., J. Bacteriol. 121: 917-922, 1975). The second and third pathway steps are catalyzed by acetohydroxy acid reductoisomerase and dehydratase, respectively. These enzymes have been characterized from a number of sources, such as for example, E. coli (Chunduru et al., Biochemistry 28:486-493, 1989); Flint et al., J. Biol. Chem. 268:14732-14742, 1993). The final two steps of the preferred isobutanol pathway occur in yeast, which can use valine as a nitrogen source and, in the process, secrete isobutanol. α-Ketoisovalerate can be converted to isobutyraldehyde by a number of keto acid decarboxylase enzymes, such as for example pyruvate decarboxylase. To prevent misdirection of pyruvate away from isobutanol production, a decarboxylase with decreased affinity for pyruvate is preferred. Suitable enzymes include two known in the art (Smit et al., Appl. Environ. Microbiol. 7:303-311, 2005); de la Plaza et al., FEMS Microbiol. Lett. 238: 367-374, 2004). Both enzymes are from strains of Lactococcus lactis and have a 50-200-fold preference for ketoisovalerate over pyruvate. Finally, a number of aldehyde reductases have been identified in yeast, many with overlapping substrate specificity. Those known to prefer branched-chain substrates over acetaldehyde include, but are not limited to, alcohol dehydrogenase VI (ADH6) and Ypr1p (Larroy et al., Biochem. J. 361:163-172, 2002); Ford et al., Yeast 19:1087-1096, 2002), both of which use NADPH as electron donor. An NADPH-dependent reductase, YqhD, active with branched-chain substrates has also been identified in E. coli (Sulzenbacher et al., J. Mol. Biol. 342: 489-502, 2004).

Another suitable pathway for converting pyruvate to isobutanol comprises the following substrate to product conversions (FIG. 1, steps a,b,c,f,g,e):
a) pyruvate to acetolactate, as catalyzed for example by acetolactate synthase,
b) acetolactate to 2,3-dihydroxyisovalerate, as catalyzed for example by acetohydroxy acid isomeroreductase,
c) 2,3-dihydroxyisovalerate to α-ketoisovalerate, as catalyzed for example by acetohydroxy acid dehydratase,
f) α-ketoisovalerate to isobutyryl-CoA, as catalyzed for example by a branched-chain keto acid dehydrogenase,
g) isobutyryl-CoA to isobutyraldehyde, as catalyzed for example by an acylating aldehyde dehydrogenase, and
e) isobutyraldehyde to isobutanol, as catalyzed for example by, a branched-chain alcohol dehydrogenase.

The first three steps in this pathway (a,b,c) are the same as those described above. The α-ketoisovalerate is converted to isobutyryl-CoA by the action of a branched-chain keto acid dehydrogenase. While yeast can only use valine as a nitrogen source, many other organisms (both eukaryotes and prokaryotes) can use valine as the carbon source as well. These organisms have branched-chain keto acid dehydrogenase (Sokatch et al. J. Bacteriol. 148: 647-652, 1981), which generates isobutyryl-CoA. Isobutyryl-CoA may be converted to isobutyraldehyde by an acylating aldehyde dehydrogenase. Dehydrogenases active with the branched-chain substrate have been described, but not cloned, in Leuconostoc and Propionibacterium (Kazahaya et al., J. Gen. Appl. Microbiol. 18: 43-55, 1972); Hosoi et al., J. Ferment. Technol. 57: 418-427, 1979). However, it is also possible that acylating aldehyde dehydrogenases known to function with straight-chain acyl-CoAs (i.e. butyryl-CoA), may also work with isobutyryl-CoA. The isobutyraldehyde is then converted to isobutanol by a branched-chain alcohol dehydrogenase, as described above for the first pathway.

Another suitable pathway for converting pyruvate to isobutanol comprises the following substrate to product conversions (FIG. 1, steps a,b,c,h,i,j,e):
a) pyruvate to acetolactate, as catalyzed for example by acetolactate synthase,
b) acetolactate to 2,3-dihydroxyisovalerate, as catalyzed for example by acetohydroxy acid isomeroreductase,
c) 2,3-dihydroxyisovalerate to α-ketoisovalerate, as catalyzed for example by acetohydroxy acid dehydratase,
h) α-ketoisovalerate to valine, as catalyzed for example by valine dehydrogenase or transaminase,
i) valine to isobutylamine, as catalyzed for example by valine decarboxylase,
j) isobutylamine to isobutyraldehyde, as catalyzed for example by omega transaminase, and
e) isobutyraldehyde to isobutanol, as catalyzed for example by, a branched-chain alcohol dehydrogenase.

The first three steps in this pathway (a,b,c) are the same as those described above. This pathway requires the addition of a valine dehydrogenase or a suitable transaminase. Valine (and or leucine) dehydrogenase catalyzes reductive amination and uses ammonia; Kₘ values for ammonia are in the millimolar range (Priestly et al., Biochem J. 261: 853-861, 1989); Vancura et al., J. Gen. Microbiol. 134: 3213-3219, 1988) Zink et al., Arch. Biochem. Biophys. 99: 72-77, 1962); Sekimoto et al. J. Biochem (Japan) 116:176-182, 1994). Transaminases typically use either glutamate or alanine as amino donors and have been characterized from a number of organisms (Lee-Peng et al., J. Bacteriol. 139:339-345, 1979); Berg et al., J. Bacteriol. 155:1009-1014, 1983). An alanine-specific enzyme may be desirable, since the generation of pyruvate from this step could be coupled to the consumption of pyruvate later in the pathway when the amine group is removed (see below). The next step is decarboxylation of valine, a reaction that occurs in valinomycin biosynthesis in Streptomyces (Garg et al., Mol. Microbiol. 46:505-517, 2002). The resulting isobutylamine may be converted to isobutyraldehyde in a pyridoxal 5′-phosphate-dependent reaction by, for example, an enzyme of the omega-aminotransferase family. Such an enzyme from Vibrio fluvialis has demonstrated activity with isobutylamine (Shin et al., Biotechnol. Bioeng. 65:206-211,1999). Another omega-aminotransferase from Alcaligenes denitrificans has been cloned and has some activity with butylamine (Yun et al., Appl. Environ. Microbiol. 70:2529-2534, 2004). In this direction, these enzymes use pyruvate as the amino acceptor, yielding alanine. As mentioned above, adverse affects on the pyruvate pool may be offset by using a pyruvate-producing transaminase earlier in the pathway. The isobutyraldehyde is then converted to isobutanol by a branched-chain alcohol dehydrogenase, as described above for the first pathway.

A fourth suitable isobutanol biosynthetic pathway comprises the substrate to product conversions shown as steps k,g,e in FIG. 1. A number of organisms are known to produce butyrate and/or butanol via a butyryl-CoA intermediate (Dürre et al., FEMS Microbiol. Rev. 17: 251-262,995); Abbad-Andaloussi et al., Microbiology, 142: 1149-1158,1996). Isobutanol production may be engineered in these organisms by addition of a mutase able to convert butyryl-CoA to isobutyryl-CoA (FIG. 1, step k). Genes for both subunits of isobutyryl-CoA mutase, a coenzyme B₁₂-dependent enzyme, have been cloned from a Streptomycete (Ratnatilleke et al., J. Biol. Chem. 274:31679-31685, 1999). The isobutyryl-CoA is converted to isobutyraldehyde (step g in FIG. 1), which is converted to isobutanol (step e in FIG. 1).

Useful for the last step of converting isobutyraldehyde to isobutanol is a new butanol dehydrogenase isolated from an environmental isolate of a bacterium identified as Achromobacter xylosoxidans, called sadB (DNA: SEQ ID NO:103, protein SEQ ID NO:104).

The preferred use in all three pathways of ketol-acid reductoisomerase (KARI) enzymes with particularly high activities is disclosed in U.S. Patent Application Publication No. 20080261230. Examples of high activity KARIs disclosed therein are those from Vibrio cholerae (DNA: SEQ ID NO:212; protein SEQ ID NO:213), Pseudomonas aeruginosa PAO1, (DNA: SEQ ID NO: 214; protein SEQ ID NO:215), and Pseudomonas fluorescens PF5 (DNA: SEQ ID NO:216; protein SEQ ID NO:217).

A person of skill in the art will be able to utilize publicly available sequences to construct relevant pathways. A listing of a representative number of genes known in the art and useful in the construction of isobutanol biosynthetic pathways are listed in Table 1. Additionally, one of skill in the art, equipped with this disclosure, will appreciate other suitable isobutanol pathways.

It is contemplated that the enzymes for an isobutanol biosynthetic pathway may have less than 100% identity to the example amino acid sequences presented herein, and still function in the biosynthetic pathway. Thus, embodiments of the present invention include host cells comprising an enzyme that catalyzes a reaction of the isobutanol biosynthetic pathway and that has at least about 70%, at least about 80%, at least about 85%, at least about 90%, at least about 95%, or at least about 98% identity to the corresponding amino acid sequences provided herein.

The term “percent identity”, as known in the art, is a relationship between two or more polypeptide sequences or two or more polynucleotide sequences, as determined by comparing the sequences. In the art, “identity” or “sequence identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humania: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991).

Preferred methods to determine identity are designed to give the best match between the sequences tested. Methods to determine identity and similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” corresponding to the alignment method labeled Clustal V (described by Higgins and Sharp, CABIOS. 5:151-153 (1989); Higgins, D. G. et al., Comput. Appl. Biosci., 8:189-191 (1992)) and found in the MegAlign™ program of the LASERGENE bioinformatics computing suite (DNASTAR Inc.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. Additionally the “Clustal W method of alignment” is available and corresponds to the alignment method labeled Clustal W (described by Higgins and Sharp, CABIOS. 5:151-153 (1989); Higgins, D. G. et al., Comput. Appl. Biosci. 8:189-191 (1992) Thompson, J. D., Higgins, D. G., and Gibson T. J. (1994) Nuc. Acid Res. 22: 4673 4680) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

### Microbial Hosts for Isobutanol Production

Microbial hosts for isobutanol production may be selected from bacteria (gram negative or gram positive), cyanobacteria, filamentous fungi and yeasts. The microbial hosts selected for the production of isobutanol should be able to convert carbohydrates to isobutanol. Suitable hosts may be selected based on criteria including: high rate of glucose utilization, availability of genetic tools for gene manipulation, and/or the ability to generate stable chromosomal alterations.

Suitable microbial hosts for the production of isobutanol include, but are not limited to, the group of Gram-positive and Gram-negative bacteria as well as fungi, preferably to members of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Klebsiella, Paenibacillus, Arthrobacter, Corynebacterium, Brevibacterium, Schizosaccharomyces, Kluyveromyces, Yarrowia, Pichia, Candida, Hansenula, and Saccharomyces. Preferred hosts include: Escherichia coli, Lactobacillus plantarum, and Saccharomyces cerevisiae.

Due to the toxicity of isobutanol to microorganisms, host strains that are more tolerant to isobutanol are particularly suitable. Selection of such tolerant hosts has been disclosed in U.S. Patent Application Publication No. 20070259411.

### Soluble Transhydrogenases

When an isobutanol biosynthetic pathway having the need for one NADH and one NADPH for every 2 molecules of pyruvate processed in the pathway to isobutanol is employed, it will be desirable if each species of reduction equivalents generated through the EDP, i.e., NADPH+H⁺ and NADH+H⁺, would be available for biosynthesis of isobutanol and not consumed in other reactions. This suggests the implementation of a flux regime that prevents any thermodynamically favored formation of NADH+H⁺ from NAD⁺ through concomitant conversion of NADPH+H⁺ into NADP⁺, catalyzed by either one or more reaction steps for the enhanced production of isobutanol.

One reaction step known to catalyze the conversion of NADPH+H⁺ into NADP⁺ through the concomitant conversion of NAD⁺ into NADH+H⁺ is carried out by a soluble transhydrogenase. For example, while Enterobacteriaceae are known to contain a soluble NADPH:NAD⁺ oxidoreductase (Sauer, U., and Canonaco, F., J. Biol. Chem., 279: 6613-6619, 2004), encoded by sthA, also referred to as udhA (Sauer, U., and Canonaco, F., supra), such an enzyme does not exist in organisms such as Saccharomyces cerevisiae which therefore cannot tolerate imbalances between catabolic NADPH production and anabolic NADPH consumption. The hypothesis of a missing soluble transhydrogenase in S. cerevisiae was further supported by the findings that a glucose-6-phosphate isomerase mutant (pgi mutant) of S. cerevisiae could not grow on glucose (Maitra, P. K., J. Bacteriol., 276: 34840-34846, 1971). However, overexpression of the soluble transhydrogenase (sthA/udhA) of E. coli, which allowed conversion of NADPH+H⁺ into NADP⁺, partially restored growth of the pgi S. cerevisiae mutant (Fiaux, J., and Cakar, Z. P., Eukaryot Cell., 2: 170-180, 2003). To date, no soluble transhydrogenase has been identified in Lactobacilli (Schomburg, D-BRENDA, The comprehensive enzyme information system, Release 2007.1., Biobase).

### By-Product Formation

It will be appreciated that reduction and preferably elimination of by-products of carbon metabolism other than carbon dioxide and isobutanol would be advantageous for production of isobutanol. For example microorganisms metabolizing sugar substrates produce a variety of by-products in a mixed acid fermentation (Moat, A. G. et al., MicrobialPhysiology, 4th edition, John Wiley Publishers, N.Y., 2002). Typical products of the bacterial mixed acid fermentation are acids and alcohols such as formic, lactic and succinic acids and ethanol and acetate. Yeast metabolizing sugar substrates produce a variety of by-products like acids and alcohols such as, but not limited to, formate, lactate, succinate, ethanol, acetate and glycerol. Formation of these byproducts during isobutanol fermentation lower the yield of isobutanol. To prevent yield loss of isobutanol the genes encoding enzyme activities corresponding to byproduct formation can be down-regulated or disrupted using methods described herein and/or known in the art.

Enzymes involved in byproduct formation in E. coli include, but are not limited to: 1) Pyruvate formate lyase (EC 2.3.1.54), encoded by pflB gene (amino acid SEQ ID NO: 259; DNA SEQ ID NO: 260), that metabolizes pyruvate to formate and acetyl-coenzyme A. Deletion of this enzyme removes the competition for pyruvate to form formate and acetyl-CoA; 2) Fumarate reductase enzyme complex (EC 1.3.99.1), encoded by frdABCD operon, that catalyses the reduction of fumarate to succinate and requires NADH; the FrdA (amino acid SEQ ID NO: 261; DNA SEQ ID NO: 262) subunit contains a covalently bound flavin adenine dinucleotide.; FrdB contains the iron-sulfur centers of the enzyme (amino acid SEQ ID NO: 263; DNA SEQ ID NO: 264); FrdC (amino acid SEQ ID NO: 265; DNA SEQ ID NO: 266) and FrdD (amino acid SEQ ID NO: 267; DNA SEQ ID NO: 268) are integral membrane proteins that bind the catalytic FrdAB domain to the cytoplasmic membrane. The function of fumarate reductase may be eliminated by deletion of any one of the subunits of frdA, B, C, or D, where deletion of frdB is preferred. Deletion of this activity removes the draw for pyruvate for its conversion to fumarate under anaerobic conditions; 3) Alcohol dehydrogenase (EC 1.2.1.10-acetaldehyde dehydrogenase and EC 1.1.1.1-alcohol dehydrogenase), enoded by adhE gene (amino acid SEQ ID NO: 269; DNA SEQ ID NO: 270), that synthesizes ethanol from acetyl-CoA in a two step reaction (both reactions are catalyzed by adhE and both reactions require NADH); and 4) Lactate dehydrogenase (EC 1.1.1.28), encoded by IdhA (amino acid SEQ ID NO: 271; DNA SEQ ID NO: 272) gene, that reduces pyruvate to lactate with oxidation of NADH. Deletion of this enzyme removes the competition for pyuruvate by this enzyme and blocks its conversion to formate and acetyl-CoA. A preferred E. coli host strain is exemplified herein (see Examples) and lacks pflB (encoding for pyruvate formate lyase), frdB (encoding for a subunit of fumarate reductase), IdhA (encoding for lactate dehydrogenase) and adhE (encoding for alcohol dehydrogenase). Any enteric bacterial gene identified as pflB, frdB, IdhA and adhE is a target for modification in the corresponding microorganism to create a strain for the production of isobutanol. In other enteric bacteria, genes encoding pyruvate formate lyase, fumarate reductase, alcohol dehydrogenase, or lactate dehydrogenase such as those having at least about 80-85%, 85%-90%, 90%-95%, or at least about 98% sequence identity to pflB, frdB, IdhA or adhE may be downregulated or disrupted.

Endogenous lactate dehydrogenase activity in lactic acid bacteria (LAB) converts pyruvate to lactate and is thus involved in byproduct formation. LAB may have one or more genes, typically one, two or three genes, encoding lactate dehydrogenase. For example, Lactobacillus plantarum has three genes encoding lactate dehydrogenase which are named IdhL2 (protein SEQ ID NO: 273, coding region SEQ ID NO: 274), IdhD (protein SEQ ID NO: 275, coding region SEQ ID NO: 276), and IdhL1 (protein SEQ ID NO: 277, coding region SEQ ID NO: 278). In other lactic acid bacteria, genes encoding lactate dehydrogenase such as those having at least about 80-85%, 85%-90%, 90%-95%, or at least about 98% sequence identity to IdhL2, IdhD, and IdhL1 may be downregulated or disrupted.

Endogenous pyruvate decarboxylase activity in yeast converts pyruvate to acetaldehyde, which is then converted to ethanol or to acetyl-CoA via acetate. Therefore, endogenous pyruvate decarboxylase activity is a target for reduction or elimination of byproduct formation. Yeasts may have one or more genes encoding pyruvate decarboylase. For example, there is one gene encoding pyruvate decarboxylase in Kluyveromyces lactis, while there are three isozymes of pyruvate decarboxylase encoded by the PDC1, PCD5, and PDC6 genes in Saccharomyces cerevisiae, as well as a pyruvate decarboxylase regulatory gene PDC2. Expression of pyruvate decarboxylase from PDC6 is minimal. In the present yeast strains the pyruvate decarboxylase activity is reduced by downregulating or disrupting at least one gene encoding a pyruvate decarboxylase, or a gene regulating pyruvate decarboxylase gene expression. For example, in S. cerevisiae the PDC1 and PDC5 genes, or all three genes, may be disrupted. Alternatively, pyruvate decarboxylase activity may be reduced by disrupting the PDC2 regulatory gene in S. cerevisiae. In other yeasts, genes encoding pyruvate decarboxylase proteins such as those having at least about 80-85%, 85%-90%, 90%-95%, or at least about 98% sequence identity to PDC1 or PDC5 may be downregulated or disrupted. Examples of yeast pyruvate decarboxylase genes or proteins that may be targeted for downregulation or disruption are listed in Table 6 (SEQ ID NOs: 280, 282, 284, 286, 288, 290, 292, 294, and 296).

Examples of yeast strains with reduced pyruvate decarboxylase activity due to disruption of pyruvate decarboxylase encoding genes have been reported such as for Saccharomyces in Flikweert et al. (Yeast (1996) 12:247-257), for Kluyveromyces in Bianchi et al. (Mol. Microbiol. (1996) 19(1):27-36), and disruption of the regulatory gene in Hohmann, (Mol Gen Genet. (1993) 241:657-666). Saccharomyces strains having no pyruvate decarboxylase activity are available from the ATCC (Accession #200027 and #200028).

### Molecular Manipulations to Produce the Host Strain

Suitable methods to express, delete/disrupt, down-regulate or up-regulate genes or a set of genes are known to one skilled in the art. Many of the methods are applicable to both bacteria and fungi including directed gene modification as well as random genetic modification followed by screening.

Typically used random genetic modification methods (reviewed in Miller, J. H. (1992) A Short Course in Bacterial Genetics. Cold Spring Harbor Press, Plainview, N.Y.) include spontaneous mutagenesis, mutagenesis caused by mutator genes, chemical mutagenesis, irradiation with UV or X-rays. Specific methods for creating mutants using radiation or chemical agents are well documented in the art. See, for example: Thomas D. Brock in Biotechnology: A Textbook of Industrial Microbiology, 2nd ed. (1989) Sinauer Associates. Additionally transposon insertions have been introduced into bacteria by phage-mediated transduction and conjugation and into bacteria by transformation. In these cases the transposon expresses a transposase in the recipient that catalyzes gene hopping from the incoming DNA to the recipient genome. The transposon DNA can be naked, incorporated in a phage or plasmid nucleic acid or complexed with a transposase. Most often the replication and/or maintenance of the incoming DNA containing the transposon is prevented, such that genetic selection for a marker on the transposon (most often antibiotic resistance) insures that each recombinant is the result of movement of the transposon from the entering DNA molecule to the recipient genome. An alternative method is one in which transposition is carried out with chromosomal DNA, fragments thereof, or a fragment thereof in vitro, and then the novel insertion allele that has been created is introduced into a recipient cell where it replaces the resident allele by homologous recombination. Transposon insertion may be performed as described in Kleckner and Botstein, J. Mol. Biol. 116:125-159, 1977), or using the Transposome™ system (Epicentre; Madison, Wis.).

Genetic modification methods include, but are not limited to, deletion of an entire gene or a portion of the gene, inserting a DNA fragment into the gene (in either the promoter or coding region) so that the protein is not expressed or expressed at lower levels, introducing a mutation into the coding region which adds a stop codon or frame shift such that a functional protein is not expressed, and introducing one or more mutations into the coding region to alter amino acids so that a non-functional or a less functional protein is expressed. Some DNA sequences surrounding the coding sequence are useful for modification methods using homologous recombination. For example, in this method gene flanking sequences are placed bounding a selectable marker gene to mediate homologous recombination whereby the marker gene replaces the gene. Also partial gene sequences and flanking sequences bounding a selectable marker gene may be used to mediate homologous recombination whereby the marker gene replaces a portion of the gene. In addition, the selectable marker may be bounded by site-specific recombination sites, so that following expression of the corresponding site-specific recombinase, the resistance gene is excised from the gene without reactivating the latter. The site-specific recombination leaves behind a recombination site which disrupts expression of the protein. The homologous recombination vector may be constructed to also leave a deletion in the gene following excision of the selectable marker, as is well known to one skilled in the art. Moreover, promoter replacement methods may be used to exchange the endogenous transcriptional control elements allowing another means to modulate expression such as described in Yuan et al. (Metab. Eng., 8:79-90, 2006).

Antisense technology is another method of molecular modification to down-regulate a gene when the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA that encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan.

In addition to down-regulate a gene and its corresponding gene product the synthesis of or stability of the transcript may be lessened by mutation. Similarly the efficiency by which a protein is translated from mRNA may be modulated by mutation. All of these methods for molecular manipulation may be readily practiced by one skilled in the art making use of the known sequences encoding proteins. DNA sequences surrounding the coding sequences are also useful in some more methods for molecular manipulations.

To up-regulate genes and subsequently increase amount and/or activity of gene products additional copies of genes may be introduced into the host. Up-regulation of the desired gene products also can be achieved at the transcriptional level through the use of a stronger promoter (either regulated or constitutive) to cause increased expression, by removing/deleting destabilizing sequences from either the mRNA or the encoded protein, or by adding stabilizing sequences to the mRNA (U.S. Pat. No. 4,910,141). Yet another approach to up-regulate a desired gene and the amount and/or activity of its gene product is to increase the translational efficiency of the encoded mRNAs by replacement of codons in the native coding gene of the gene product with those for optimal gene expression and translation in the selected host microorganism.

Tables 2-4 provide a listing of genes from various organisms that may be genetically manipulated to modify glucose metabolic pathways according to the teachings herein.

### Isolation of Homologous Genes

In the process of building an isobutanol pathway or in modifying a glucose metabolic pathway it may be useful to isolate gene homologs based on structure, sequence and function. Methods for identifying and isolating genetic homologs on the basis of sequence are common and well known in the art and include for example 1) methods of nucleic acid hybridization; 2) methods of DNA and RNA amplification, as exemplified by various uses of nucleic acid amplification technologies (e.g., polymerase chain reaction (PCR), Mullis et al., U.S. Pat. No. 4,683,202; ligase chain reaction (LCR), Tabor, S. et al., Proc. Acad. Sci. USA 82:1074. 1985; or strand displacement amplification (SDA), Walker, et al., Proc. Natl. Acad. Sci. U.S.A., 89:392, 1992); and 3) methods of library construction and screening by complementation.

a) Nucleic acid hybridization. For example, genes encoding similar proteins or polypeptides to genes provided herein could be isolated directly by using all or a portion of the instant nucleic acid fragments as DNA hybridization probes to screen libraries from any desired organism using methodology well known to those skilled in the art. Specific oligonucleotide probes based upon the disclosed nucleic acid sequences can be designed and synthesized by methods known in the art (Maniatis, supra). Moreover, the entire sequences can be used directly to synthesize DNA probes by methods known to the skilled artisan (e.g., random primers DNA labeling, nick translation or end-labeling techniques), or RNA probes using available in vitro transcription systems. In addition, specific primers can be designed and used to amplify a part of (or full-length of) the instant sequences. The resulting amplification products can be labeled directly during amplification reactions or labeled after amplification reactions, and used as probes to isolate full-length DNA fragments by hybridization under conditions of appropriate stringency. The basic components of a nucleic acid hybridization test include a probe, a sample suspected of containing the gene or gene fragment of interest, and a specific hybridization method. Probes are typically single-stranded nucleic acid sequences that are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

Hybridization methods are well defined. Typically the probe and sample must be mixed under conditions that will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed. Optionally, a chaotropic agent may be added. The chaotropic agent stabilizes nucleic acids by inhibiting nuclease activity. Furthermore, the chaotropic agent allows sensitive and stringent hybridization of short oligonucleotide probes at room temperature (Van Ness and Chen, Nucl. Acids Res., 19:5143-5151, 1991). Suitable chaotropic agents include guanidinium chloride, guanidinium thiocyanate, sodium thiocyanate, lithium tetrachloroacetate, sodium perchlorate, rubidium tetrachloroacetate, potassium iodide and cesium trifluoroacetate, among others. Typically, the chaotropic agent will be present at a final concentration of about 3 M. If desired, one can add formamide to the hybridization mixture, typically 30-50% (v/v).

Various hybridization solutions can be employed. Typically, these comprise from about 20 to 60% volume, preferably 30%, of a polar organic solvent. A common hybridization solution employs about 30-50% v/v formamide, about 0.15 to 1 M sodium chloride, about 0.05 to 0.1 M buffers (e.g., sodium citrate, Tris-HCl, PIPES or HEPES (pH range about 6-9)), about 0.05 to 0.2% detergent (e.g., sodium dodecylsulfate), or between 0.5-20 mM EDTA, FICOLL (Pharmacia Inc.) (about 300-500 kD), polyvinylpyrrolidone (about 250-500 kdal) and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents that include a variety of polar water-soluble or swellable agents (e.g., polyethylene glycol), anionic polymers (e.g., polyacrylate or polymethylacrylate) and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

b) PCR-type amplification techniques: typically in PRC-type amplification methods the primers have different sequences and are not complementary to each other. Depending on the desired test conditions, the sequences of the primers should be designed to provide for both efficient and faithful replication of the target nucleic acid. Methods of PCR primer design are common and well known in the art (Thein and Wallace, “The use of oligonucleotides as specific hybridization probes in the Diagnosis of Genetic Disorders”, in Human Genetic Diseases: A Practical Approach, K. E. Davis Ed., (1986) pp 33-50, IRL: Herndon, Va.; and Rychlik, W., In Methods in Molecular Biology, White, B. A. Ed., (1993) Vol. 15, pp 31-39, PCR Protocols: Current Methods and Applications. Humania: Totowa, N.J.).

Generally two short segments of the described sequences may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the described nucleic acid fragments, and the sequence of the other primer takes advantage of the presence of the polyadenylic acid tracts to the 3′ end of the mRNA precursor encoding some microbial genes.

Alternatively, the second primer sequence may be based upon sequences derived from the cloning vector. For example, the skilled artisan can follow the RACE protocol (Frohman et al., Proc. Natl. Acad. Sci., USA, 85:8998, 1988) to generate cDNAs by using PCR to amplify copies of the region between a single point in the transcript and the 3′ or 5′ end. Primers oriented in the 3′ and 5′ directions can be designed from the instant sequences. Using commercially available 3′RACE or 5′RACE systems (e.g., BRL, Gaithersburg, Md.), specific 3′ or 5′ cDNA fragments can be isolated (Ohara et al., Proc. Natl. Acad. Sci., USA 86: 5673, 1989; and Loh et al., Science, 243: 217, 1989).

c) Library construction and screening by complementation: Genomic libraries can also be used to identify functional homologs. For example, genomic DNA from pure or mixed microbial cultures can be purified and fragmented by restriction digest or physical shearing into short segments typically 500 to 5 kb in length. These DNA fragments can be subcloned into bacterial or yeast expression vectors and expressed in host cells. Complementation can then be used to screen and identify functional homologs that either restore growth or a phenotypic condition.

Within the context of the present invention, it may be useful to modulate the expression of metabolic pathways by any one of the methods described above. For example, the present invention provides methods whereby genes encoding key enzymes in the EDP are introduced into E. coli, Lactobacilli and yeasts for upregulation of these pathways. It will be particularly useful to express these genes in bacteria or yeasts that do not have the EDP pathway and coordinate the expression of these genes, to maximize production of isobutanol using various means for metabolic engineering of the host organism.

Strains can then be selected and assayed for reduced or increased enzyme expression. If the organism has a means of genetic exchange then genetic crosses may be performed to verify that the effect is due to the observed alteration in the genome.

### Molecular Manipulations in Bacterial Host Cells

Molecular manipulation of genes may be carried out directly in the bacterial chromosome by any of the methods described herein and/or known to those skilled in the art. Briefly PCR and/or cloning methods well known to one skilled in the art may be used to construct a modified chromosomal segment. The segment may include a deletion, an insertion or a point mutation of a gene or a regulatory region. Alternatively the modification may include a gene encoding a new enzyme activity or an additional copy of a gene encoding an endogenous enzyme activity. Depending on the modification the engineered segment may express, delete/disrupt, down-regulate or up-regulate a gene or set of genes. Insertion of the engineered chromosomal segment may be by any method known to one skilled in the art, such as by phage transduction, conjugation, or plasmid introduction or non-plasmid double or single stranded DNA introduction followed by homologous recombination. Homologous recombination is enabled by a method that introduces homologous sequences to the modified chromosomal segment. The homologous sequences naturally flank the chromosomal segment in the bacterial chromosome, thus providing sequences to direct recombination. The flanking homologous sequences are sufficient to support homologous recombination, as described in Lloyd, R. G., and K. B. Low (Homologous recombination, p. 2236-2255; In F. C. Neidhardt, ed., Escherichia coli and Salmonella: Cellular and Molecular Biology, 1996, ASM Press, Washington, D.C.). Typically homologous sequences used for homologous recombination are over 1 kb in length, but may be as short as 50 or 100 bp. DNA fragments containing the engineered chromosomal segment and flanking homologous sequences may be prepared with defined ends, such as by restriction digestion, or using a method that generates random ends such as sonication. In either case, the DNA fragments carrying the engineered chromosomal segment may be introduced into the target host cell by any DNA uptake method, including for example, electroporation, a freeze-thaw method, or using chemically competent cells. The DNA fragment undergoes homologous recombination which results in replacement of the endogenous chromosomal region of the target host with the engineered chromosomal segment.

A plasmid may be used to carry the engineered chromosomal segment and flanking sequences into the target host cell for insertion. Typically a non-replicating plasmid is used to promote integration. Introduction of plasmid DNA is as described above.

In the case of E. coli, homologous recombination may be enhanced by use of bacteriophage homologous recombination systems, such as the bacteriophage lambda Red system (Datsenko and Wanner, Proc. Natl. Acad. Sci. USA, 97: 6640-6645, 2000) and (Ellis et al., Proc. Natl. Acad. Sci. USA, 98: 6742-6746, 2001) or the Rac phage RecE/RecT system (Zhang et al., Nature Biotechnol., 18:1314-1317, 2000). In any of these methods, the homologous recombination results in replacement of the endogenous chromosomal region of the target host with the engineered chromosomal segment.

Recipient strains with successful insertion of the engineered chromosomal segment may be identified using a marker. Either screening or selection markers may be used, with selection markers being particularly useful. For example, an antibiotic resistance marker may be present in the engineered chromosomal segment, such that when it is transferred to a new host; cells receiving the engineered chromosomal segment can be readily identified by growth on the corresponding antibiotic. Alternatively a screening marker may be used, which is one that confers production of a product that is readily detected. If it is desired that the marker not remain in the recipient strain, it may subsequently be removed such as by using site-specific recombination. In this case site-specific recombination sites are located 5′ and 3′ to the marker DNA sequence such that expression of the recombinase will cause deletion of the marker. Once the mutations have been created the cells must be screened for absence of these specific genes. A number of methods may be used to analyze for this purpose.

Another method of molecular manipulation to up-regulate a gene or set of genes includes, but is not limited to introducing additional copies of selected genes into the host cell on multicopy plasmids. Vectors or cassettes useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention. Particularly useful for expression in E. coli are promoters including, but not limited to, lac, ara, tet, trp, IP_{L}, IP_{R}, T7, tac, and trc. Termination control regions may also be derived from various genes native to the preferred hosts. Optionally, a termination site may be unnecessary, however, it is most preferred if included.

The genus Lactobacillus belongs to a group of gram positive bacteria that make up the lactic acid bacteria. Many plasmids and vectors used in the transformation of Bacillus subtilis, Enterococcus spp., and lactic acid bacteria may be used for Lactobacillus. Shuttle vectors with two origins of replication and selectable markers which allow for replication and selection in both Escherichia coli and Lactobacillus plantarum are also suitable for this invention. This allows for cloning in E. coli and expression in L. plantarum. Non-limiting examples of suitable vectors include pAMβ1 and derivatives thereof, for example pTRKL1 (LeBlanc and Lee, J. Bacteriol., 157:445-453, 1984); O′Sullivan and Klaenhammer, Gene, 137:227-231, 1993); pMBB1 and pHW800, a derivative of pMBB1 (Wyckoff et al. Appl. Environ. Microbiol. 62:1481-1486, 1996); pMG1, a conjugative plasmid (Tanimoto et al., J. Bacteriol., 184:5800-5804, 2002); pNZ9520 (Kleerebezem et al., Appl. Environ. Microbiol., 63:4581-4584, 1997); pAM401 (Fujimoto et al., Appl. Environ. Microbiol., 67:1262-1267, 2001); and pAT392 (Arthur et al., Antimicrob. Agents Chemother., 38:1899-1903, 1994). Several plasmids from L. plantarum have also been reported (e.g., van Kranenburg R, Golic N, Bongers R, Leer R J, de Vos W M, Siezen R J, Kleerebezem M. Appl. Environ. Microbiol., 7: 1223-1230, 2005). Initiation control regions or promoters, which are useful to drive expression of a coding region in order to up-regulate gene expression in L. plantarum are familiar to those skilled in the art. Some examples include the amy, apr, and npr promoters; nisA promoter (useful for expression Gram-positive bacteria (Eichenbaum et al. Appl. Environ. Microbiol. 64:2763-2769, 1998); and the synthetic P11 promoter (useful for expression in L. plantarum, Rud et al., Microbiology, 152:1011-1019, 2006). In addition, native promoters, such as the IdhL1 promoter, are useful for expression of chimeric genes in L. plantarum.

Deletion/disruption and down-regulation of a gene or set of genes may be achieved by many methods in L. plantarum. One particular method suitable for this invention utilizes a two-step homologous recombination procedure to yield unmarked deletions as has been previously described (Ferain et al., J. Bacteriol., 176: 596, 1994). The procedure utilizes a shuttle vector in which two segments of DNA containing sequences upstream and downstream of the intended deletion are cloned to provide the regions of homology for the two genetic crossovers. After the plasmid is introduced into the cell, an initial homologous crossover integrates the plasmid into the chromosome. The second crossover event yields either the wild type sequence or the intended gene deletion, which can be screened for by PCR. This procedure may also be used by those skilled in the art for chromosomal integrations and chromosomal site-specific mutagenesis.

### Molecular Manipulations in Fungal Host Cells

Any bacterial or fungal gene or set of genes of interest may be expressed and up-regulated in a yeast host cell in order to obtain and increase amount and/or activity of the respective gene product. Many molecular methods used for such manipulations are applicable to both bacteria and fungi. However, fungal host cells contain sub-structures, e.g. organelles that provide distinct environments to proteins.

Consequently, the term “heterologous gene” or “heterologous protein” additionally comprises, but is not limited to, a gene and its gene product that is expressed in a manner differently from a corresponding endogenous gene or gene product, e.g. if the gene product targets a compartment different than the corresponding endogenous gene product in the cell. For example in yeast, endogenous ketol acid reductoisomerase is encoded by ILV5 in the nucleus and the expressed ILV5 protein has a mitochondrial targeting signal sequence such that the protein is localized in the mitochondrion. It is desirable to express ILV5 activity in the cytosol for participation in biosynthetic pathways that are localized in the cytosol. Cytosolic expression of ILV5 in yeast is heterologous expression since the native protein is localized in the mitochondria. For example, heterologous expression of the Saccharomyces cerevisiae ILV5 in S. cerevisiae is obtained by expressing the S. cerevisiae ILV5 coding region with the mitochondrial targeting signal removed, such that the protein remains in the cytosol.

Molecular manipulation for expressing or up-regulating a gene or set of genes is achieved by transforming the fungal cell with a gene or set of genes comprising a sequence encoding a given protein or set of proteins. The coding region to be expressed may be codon optimized for the target host cell, as well known to one skilled in the art. Methods for molecular manipulation of expression in yeast are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression of genes in yeast typically utilizes a promoter, operably linked to a coding region of interest, and a transcriptional terminator. A number of yeast promoters can be used in constructing expression cassettes for genes in yeast, including, but not limited to promoters derived from the following genes: CYC1, HIS3, GAL1, GAL10, ADH1, PGK, PHO5, GAPDH, ADC1, TRP1, URA3, LEU2, ENO, TPI, CUP1, FBA, GPD, GPM, TEF1, and AOX1. Suitable transcriptional terminators include, but are not limited to FBAt, GPDt, GPMt, ERG10t, GAL1t, CYC1, and ADH1.

Suitable promoters, transcriptional terminators, and coding regions may be cloned into E. coli-yeast shuttle vectors, and transformed into yeast cells. These vectors allow strain propagation in both E. coli and yeast strains. Typically the vector used contains a selectable marker and sequences allowing autonomous replication or chromosomal integration in the desired host. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μorigin of replication, and a marker for nutritional selection. The selection markers for these four vectors are His3 (vector pRS423), Trp1 (vector pRS424), Leu2 (vector pRS425) and Ura3 (vector pRS426). Construction of expression vectors with a chimeric gene encoding the described protein coding region may be performed by either standard molecular cloning techniques in E. coli or by the gap repair recombination method in yeast.

The gap repair cloning approach takes advantage of the highly efficient homologous recombination in yeast. Typically, a yeast vector DNA is digested (e.g., in its multiple cloning site) to create a “gap” in its sequence. A number of insert DNAs of interest are generated that contain a ≧21 by sequence at both the 5′ and the 3′ ends that sequentially overlap with each other, and with the 5′ and 3′ terminus of the vector DNA. For example, to construct a yeast expression vector for the desired gene a yeast promoter and a yeast terminator are selected for the expression cassette. The promoter and terminator are amplified from the yeast genomic DNA, and Gene X is either PCR amplified from its source organism or obtained from a cloning vector comprising the desired gene sequence. There is at least a 21 by overlapping sequence between the 5′ end of the linearized vector and the promoter sequence, between the promoter and the desired gene, between Gene X and the terminator sequence, and between the terminator and the 3′ end of the linearized vector. The “gapped” vector and the insert DNAs are then co-transformed into a yeast strain and plated on the medium containing the appropriate compound mixtures that allow complementation of the nutritional selection markers on the plasmids. The presence of correct insert combinations can be confirmed by PCR mapping using plasmid DNA prepared from the selected cells. The plasmid DNA isolated from yeast (usually low in concentration) can then be transformed into an E. coli strain, (e.g. TOP10 or DH10B), followed by mini preps and restriction mapping to further verify the plasmid construct. Finally the construct can be verified by sequence analysis.

Like the gap repair technique, integration into the yeast genome also takes advantage of the homologous recombination system in yeast. Typically, a cassette containing a coding region plus control elements (promoter and terminator) and auxotrophic marker is PCR-amplified with a high-fidelity DNA polymerase using primers that hybridize to the cassette and contain 40-70 base pairs of sequence homology to the regions 5′ and 3′ of the genomic area where insertion is desired. The PCR product is then transformed into yeast and plated on medium containing the appropriate compound mixtures that allow selection for the integrated auxotrophic marker. For example, to integrate “Gene X” into chromosomal location “Y”, the promoter-coding regionX-terminator construct is PCR amplified from a plasmid DNA construct and joined to an autotrophic marker (such as URA3) by either SOE PCR or by common restriction digests and cloning. The full cassette, containing the promoter-coding region X-terminator-URA3 region, is PCR amplified with primer sequences that contain 40-70 by of homology to the regions 5′ and 3′ of location “Y” on the yeast chromosome. The PCR product is transformed into yeast and selected on growth media lacking uracil. Transformants can be verified either by colony PCR or by direct sequencing of chromosomal DNA.

Molecular manipulation for down-regulation or deletion/disruption of a gene or set of genes may be achieved in any yeast cell that is amenable to genetic manipulation. Examples include yeasts of Saccharomyces, Schizosaccharomyces, Hansenula, Candida, Kluyveromyces, Yarrowia and Pichia. Suitable strains include, but are not limited to, Saccharomyces cerevisiae, Schizosaccharomyces pombe, Kluyveromyces lactis, Kluyveromyces thermotolerans, Candida glabrata, Candida albicans, Pichia stipitis and Yarrowia lipolytica. Particularly suitable is Saccharomyces cerevisiae.

In any of these yeasts, any endogenous gene or set of genes may be a target for deletion/disruption and/or down-regulation including, for example, phosphofructokinase (PFK1). At least one gene encoding an endogenous phosphofructokinase protein is disrupted, and two or more genes encoding endogenous phosphofructokinase proteins may be disrupted, to reduce phosphofructokinase protein expression.

Because fungal genes are well known, and because of the prevalence of genomic sequencing, additional suitable PFK1 may be readily identified for deletion/disruption and down-regulation by one skilled in the art on the basis of sequence similarity using bioinformatics approaches. Typically BLAST (described above) searching of publicly available databases with known PFK1 amino acid sequences, such as those provided herein, is used to identify PFK1 and their encoding sequences that may be targeted for inactivation in the present strains. For example, PFK1 proteins having amino acid sequence identities of at least about 70-75%, 75%-80%, 80-85%, 85%-90%, 90%-95% and at least about 98% sequence identity to any of the PFK1 proteins in Table 4 (SEQ ID NOs:164,166,172,174, and 176) may be inactivated in the present strains. Identities are based on the ClustalW method of alignment using the default parameters of GAP PENALTY=10, GAP LENGTH PENALTY=0.1, and Gonnet 250 series of protein weight matrix.

In addition, mutagenesis can also be used for expression, up-regulation, down-regulation or deletion/disruption of a gene or set of genes in fungal host cells. Methods for creating genetic mutations are common and well known in the art and may be applied to the exercise of creating mutants. Commonly used random genetic modification methods (reviewed in Methods in Yeast Genetics, 2005, Cold Spring Harbor Laboratory Press, Cold Spring Harbor, N.Y.) include spontaneous mutagenesis, mutagenesis caused by mutator genes, chemical mutagenesis, irradiation with UV or Xrays, or transposon mutagenesis. Chemical mutagenesis of yeast commonly involves treatment of yeast cells with one of the following DNA mutagens: ethyl methanesulfonate (EMS), nitrous acid, diethyl sulfate, or N-methyl-N′-nitro-30 N-nitroso-guanidine (MNNG). These methods of mutagenesis have been reviewed in Spencer et al (Mutagenesis in Yeast, 1996, Yeast Protocols: Methods in Cell and Molecular Biology. Humana Press, Totowa, N.J.). Chemical mutagenesis with EMS may be performed as described in Methods in Yeast Genetics, 2005, Cold Spring Harbor Laboratory Press, Cold Spring Harbor, N.Y. Irradiation with ultraviolet (UV) light or X-rays can also be used to produce random mutagenesis in yeast cells. The primary effect of mutagenesis by UV irradiation is the formation of pyrimidine dimers which disrupt the fidelity of DNA replication. Protocols for UV-mutagenesis of yeast can be found in Spencer et al (Mutagenesis in Yeast, 1996, Yeast Protocols: Methods in Cell and Molecular Biology. Humana Press, Totowa, N.J.). Introduction of a mutator phenotype can also be used to generate random chromosomal mutations in yeast. Common mutator phenotypes can be obtained through disruption of one or more of the following genes: PMS1, MAGI, RAD18 or RAD51. Restoration of the non-mutator phenotype can be easily obtained by insertion of the wildtype allele. Collections of modified cells produced from any of these or other known random mutagenesis processes may be screened for reduced enzyme activity.

Construction of an E. coli Production Host of the Invention

Particularly suitable in the present invention are members of the enteric class of bacteria. Enteric bacteria are members of the family Enterobacteriaceae and include such members as Escherichia, Salmonella, and Shigella.

One aspect of the invention includes optimization of isobutanol production in E. coli by an enhanced EDP. Methods for optimization of isobutanol production by an enhanced EDP in E. coli include: 1) expression and up-regulation of a set of genes that encodes enzymes of an isobutanol production pathway; 2) expression and/or up-regulation of a gene or set of genes the encodes preferred enzyme(s) of an enhanced EDP 3) decreasing flux through competing carbon-metabolizing pathways in order to achieve e.g. a diminished EMP and a diminished oxidative PPP using, for example, molecular manipulations described herein to disrupt and/or down-regulate a gene or set of genes and the corresponding gene product(s) 4) preventing the loss of carbon and redox metabolites like e.g. NADPH through NAD reduction by disrupting or down-regulating the soluble transhydrogenase reaction (EC number 1.6.1.1).

Methods for gene expression and creation of mutations in Enterobacteriaceae such as E. coli are well known in the art. Suitable isobutanol pathway genes and genetic constructs are provided herein, as elaborated in the section on “isobutanol biosynthetic pathways” and in the Examples. The genes as well as the plasmids and regulatory backbone can easily be replaced by and/or augmented with alternatives by one skilled in the art using methods and tools known and/or described herein, in order to provide an alternative functional isobutanol pathway. Genes of an isobutanol biosynthetic pathway may be isolated from various sources and cloned into various vectors as described in Examples 1, 2, 9, 10, 11, 12, and 14 of U.S. Patent Application Publication No. 20070092957, incorporated herein by reference.

Since E. coli possesses all the required genes for a functional EDP in its genome, up-regulation of endogenous EDP genes results in an enhanced EDP. Alternatively, an enhanced EDP can be accomplished through the expression and up-regulation of heterologous genes of the set of genes encoding EDP activities, comprising glucose-6-phosphate dehydrogenase reaction (EC 1.1.1.49), 6-phosphogluconolactonase reaction (EC 3.1.1.31), phosphogluconate dehydratase reaction (EC 4.2.1.12), and 2-dehydro-3-deoxy-phosphogluconate aldolase reaction (EC 4.1.2.14) as elaborated on in the following. Activity from heterologous EDP genes can either replace or augment activity from endogenous EDP genes.

Genes that encode EDP activities such as glucose-6-phosphate dehydrogenase reaction (EC 1.1.1.49) are preferably chosen from either Aspergillus niger, specifically GenBank No: CAA61194.1 (SEQ ID NO:117), Aspergillus nidulans, specifically GenBank No: XP_{—}660585.1 (SEQ ID NO:119), Schizosaccharomyces pombe, specifically GenBank Nos: NP_{—}587749.1 (SEQ ID NO:123), or NP_{—}593614.1 (SEQ ID NO:124), or NP_{—}593344.2 (SEQ ID NO:121), Escherichia coli, specifically GenBank No: NP_{—}416366.1 (SEQ ID NO:127), Lactobacillus plantarum, specifically GenBank No: NP_{—}786078.1 (SEQ ID NO:131), or Saccharomyces cerevisiae, specifically GenBank No: NP_{—}014158.1 (SEQ ID NO:133), and are referred to as edp1.

Genes that encode EDP activities such as 6-phosphogluconolactonase reaction (EC 3.1.1.31) are preferably chosen from either Escherichia coli, specifically GenBank No: NP_{—}415288.1 (SEQ ID NO:105), Lactobacillus plantarum, specifically GenBank No: NP_{—}785709.1 (SEQ ID NO:111), Saccharomyces cerevisiae, specifically GenBank No: NP_{—}011764.1 (SEQ ID NO:107) NP_{—}012033.2 (SEQ ID NO:190), and Zymomonas mobilis, specifically GenBank No: YP_{—}163213.1 (SEQ ID NO:113) and AE008692 (SEQ ID NO:113) and are referred to as edp2.

Genes that encode EDP activities such as phosphogluconate dehydratase reaction (EC 4.2.1.12) are preferably chosen from either Zymomonas mobilis, specifically GenBank No: YP_{—}162103.1 (SEQ ID NO:135), Pseudomonas putida, specifically GenBank No: NP_{—}743171.1 (SEQ ID NO:137), or Escherichia coli, specifically GenBank No: NP_{—}416365.1 (SEQ ID NO:139), and are referred to as edp3.

Genes that encode EDP activities such as 2-dehydro-3-deoxy-phosphogluconate aldolase reaction (EC 4.1.2.14) are preferably chosen from either Azotobacter vinelandii, specifically GenBank Nos ZP_{—}00417447.1 (SEQ ID NO:194), ZP_{—}00415409.1 (SEQ ID NO: 196), ZP_{—}00416840.1 (SEQ ID NO:198), or ZP_{—}00419301.1 (SEQ ID NO:200), Pseudomonas putida, specifically GenBank No: NP_{—}743185.1 (SEQ ID NO:202), Pseudomonas fluorescens, specifically GenBank No: YP_{—}261692.1 (SEQ ID NO:204), Zymomonas mobilis, specifically GenBank No: YP_{—}162732.1 (SEQ ID NO:206), or Escherichia coli, specifically GenBank No: NP_{—}416364.1 (SEQ ID NO:208), and are referred to as edp4.

Decreasing flux through competing carbon-metabolizing pathways is achieved through e.g. the disruption or down-regulation of EMP- and PPP-specific genes and their gene products. To diminish oxidative PPP, e.g. the 6-phosphogluconate dehydrogenase activity in E. coli encoded by the gnd gene (SEQ ID NO: 143), is down-regulated or disrupted. By this means Zhao et al. (Zhao, Baba et al., Appl Microbiol Biotechnol 64(1): 91-8) were able to increase relative flux through EDP from 0% to 10%.

To decrease flux through EMP in E. coli, pgi (SEQ ID NO: 155) is down-regulated or deleted (Canonaco, Hess et al. 2001, FEMS Microbiol Lett 204(2): 247-52). Alternatively, flux through 6-phosphofructokinase reaction, converting fructose-6-phosphate to fructose-1,6-bisphosphate is reduced or completely eliminated. In E. coli, this reaction is catalyzed by two iso-enzymes, encoded by the genes pfkA (SEQ ID NO: 165) and pfkB (SEQ ID NO: 163). Diminishing or completely eliminating flux through 6-phosphofructokinase reaction is achieved by deletion and/or down-regulation of at least one, preferably both of these iso-enzymes. Alternatively, rate of the fructose-bisphosphate aldolase reaction is diminished or completely eliminated through the down-regulation and/or deletion of at least one, preferably both iso-enzymes known to catalyze the fructose-bisphosphate aldolase reaction, encoded by the genes fbaA (SEQ ID NO:179) and fbaB (SEQ ID NO: 177) in E. coli. However, reduced or eliminated fructose-bisphosphate aldolase reaction leads to elevated levels of fructose-1,6-bisphosphate in the cells that was found to activate flux through EMP enzymes, but inhibit flux through competing pathways like e.g. PPP and EDP (Kirtley, M. E. et al., Mol. Cell. Biochem., 18: 141-149, 1977). Consequently, down-regulation and/or deletion of 6-phosphofructokinase reaction, converting fructose-6-phosphate to fructose-1,6-bisphosphate, in conjunction with diminishing or complete elimination of the fructose-bisphosphate aldolase reaction at the same time is desirable. In E. coli, this is achieved by the down-regulation and/or deletion of at least two, preferably more genes of the gene set comprising pfkA, pfkB, fbaA and fbaB. Decreasing flux through the competing carbon-metabolizing pathways EMP- and oxidative PPP can be achieved through the disruption or down-regulation of one or more genes of the gene set comprising pgi, pfkA, pfkB, fbaA, fbaB and gnd.

Another aspect of the invention addresses optimization of isobutanol production through reducing the use of redox metabolites like NADPH in reactions other than isobutanol biosynthesis. This is achieved by for example reducing or completely eliminating flux through soluble transhydrogenase, in E. coli achieved through the down-regulation and/or disruption of the sthA gene.

E. coli genotypes provided herein include the following, with and without up-regulated endogenous EDP pathway genes: E. coli K12 pTrc99A::budB-ilvC-ilvD-kivD-sadB, E. coli K12 ΔpfkA pTrc99A::budB-ilvC-ilvD-kivD-sadB, E. coli K12 ΔpfkB pTrc99A::budB-ilvC-ilvD-kivD-sadB, E. coli K12 ΔfbaA pTrc99A::budB-ilvC-ilvD-kivD-sadB, E. coli K12 ΔfbaB pTrc99A::budB-ilvC-ilvD-kivD-sadB, E. coli K12 ΔsthA pTrc99A::budB-ilvC-ilvD-kivD-sadB, E. coli K12 Δgnd pTrc99A::budB-ilvC-ilvD-kivD-sadB, E. coli K12 pCL1925-edp1 pTrc99A::budB-ilvC-ilvD-kivD-sadB, E. coli K12 pCL1925-edp2 pTrc99A::budB-ilvC-ilvD-kivD-sadB, E. coli K12 pCL1925-edp3 pTrc99A::budB-ilvC-ilvD-kivD-sadB, E. coli K12 pCL1925-edp4 pTrc99A::budB-ilvC-ilvD-kivD-sadB, E. coli K12 ΔpfkA ΔpfkB pTrc99A::budB-ilvC-ilvD-kivD-sadB, E. coli K12 ΔpfkA ΔfbaA pTrc99A::budB-ilvC-ilvD-kivD-sadB, E. coli K12 ΔpfkA ΔfbaB pTrc99A::budB-ilvC-ilvD-kivD-sadB, E. coli K12 ΔpfkA ΔsthA pTrc99A::budB-ilvC-ilvD-kivD-sadB, E. coli K12 ΔpfkA Δgnd pTrc99A::budB-ilvC-ilvD-kivD-sadB, E. coli K12 ΔpfkA pCL1925-edp1 pTrc99A::budB-ilvC-ilvD-kivD-sadB, E. coli K12 ΔpfkA pCL1925-edp2 pTrc99A::budB-ilvC-ilvD-kivD-sadB, E. coli K12 ΔpfkA pCL1925-edp3 pTrc99A::budB-ilvC-ilvD-kivD-sadB, E. coli K12 ΔpfkA pCL1925-edp4 pTrc99A::budB-ilvC-ilvD-kivD-sadB, E. coli K12 ΔpfkA ΔpfkB ΔfbaA pTrc99A::budB-ilvC-ilvD-kivD-sadB, E. coli K12 ΔpfkA ΔpfkB ΔfbaB pTrc99A::budB-ilvC-ilvD-kivD-sadB, E. coli K12 ΔpfkA ΔpfkB ΔsthA pTrc99A::budB-ilvC-ilvD-kivD-sadB, E. coli K12 ΔpfkA ΔpfkB Δgnd pTrc99A::budB-ilvC-ilvD-kivD-sadB, E. coli K12 ΔpfkA ΔpfkB pCL1925-edp1 pTrc99A::budB-ilvC-ilvD-kivD-sadB, E. coli K12 ΔpfkA ΔpfkB pCL1925-edp2 pTrc99A::budB-ilvC-ilvD-kivD-sadB, E. coli K12 ΔpfkA ΔpfkB pCL1925-edp3 pTrc99A::budB-ilvC-ilvD-kivD-sadB, E. coli K12 ΔpfkA ΔpfkB pCL1925-edp4 pTrc99A::budB-ilvC-ilvD-kivD-sadB, E. coli K12 ΔpfkA ΔpfkB ΔfbaA ΔfbaB pTrc99A::budB-ilvC-ilvD-kivD-sadB, E. coli K12 ΔpfkA ΔpfkB ΔfbaA ΔsthA pTrc99A::budB-ilvC-ilvD-kivD-sadB, E. coli K12 ΔpfkA ΔpfkB ΔfbaA Δgnd pTrc99A::budB-ilvC-ilvD-kivD-sadB, E. coli K12 ΔpfkA ΔpfkB ΔfbaA pCL1925-edp1 pTrc99A::budB-ilvC-ilvD-kivD-sadB, E. coli K12 ΔpfkA ΔpfkB ΔfbaA pCL1925-edp2 pTrc99A::budB-ilvC-ilvD-kivD-sadB, E. coli K12 ΔpfkA ΔpfkB ΔfbaA pCL1925-edp3 pTrc99A::budB-ilvC-ilvD-kivD-sadB, E. coli K12 ΔpfkA ΔpfkB ΔfbaA pCL1925-edp4 pTrc99A::budB-ilvC-ilvD-kivD-sadB, E. coli K12 ΔpfkA ΔpfkB ΔfbaA ΔfbaB ΔsthA pTrc99A::budB-ilvC-ilvD-kivD-sadB, E. coli K12 ΔpfkA ΔpfkB ΔfbaA ΔfbaB Δgnd pTrc99A::budB-ilvC-ilvD-kivD-sadB, E. coli K12 ΔpfkA ΔpfkB ΔfbaA ΔfbaB pCL1925-edp1 pTrc99A::budB-ilvC-ilvD-kivD-sadB, E. coli K12 ΔpfkA ΔpfkB ΔfbaA ΔfbaB pCL1925-edp2 pTrc99A::budB-ilvC-ilvD-kivD-sadB, E. coli K12 ΔpfkA ΔpfkB ΔfbaA ΔfbaB pCL1925-edp3 pTrc99A::budB-ilvC-ilvD-kivD-sadB, E. coli K12 ΔpfkA ΔpfkB ΔfbaA ΔfbaB pCL1925-edp4 pTrc99A::budB-ilvC-ilvD-kivD-sadB, E. coli K12 ΔpfkA ΔpfkB ΔfbaA ΔfbaB ΔsthA Δgnd pTrc99A::budB-ilvC-ilvD-kivD-sadB, E. coli K12 ΔpfkA ΔpfkB ΔfbaA ΔfbaB ΔsthA pCL1925-edp1 pTrc99A::budB-ilvC-ilvD-kivD-sadB, E. coli K12 ΔpfkA ΔpfkB ΔfbaA ΔfbaB ΔsthA pCL1925-edp2 pTrc99A::budB-ilvC-ilvD-kivD-sadB, E. coli K12 ΔpfkA ΔpfkB ΔfbaA ΔfbaB ΔsthA pCL1925-edp3 pTrc99A::budB-ilvC-ilvD-kivD-sadB, E. coli K12 ΔpfkA ΔpfkB ΔfbaA ΔfbaB ΔsthA pCL1925-edp4 pTrc99A::budB-ilvC-ilvD-kivD-sadB, E. coli K12 ΔpfkA ΔpfkB ΔfbaA ΔfbaB ΔsthA Δgnd pCL1925-edp1 pTrc99A::budB-ilvC-ilvD-kivD-sadB, E. coli K12 ΔpfkA ΔpfkB ΔfbaA ΔfbaB ΔsthA Δgnd pCL1925-edp2 pTrc99A::budB-ilvC-ilvD-kivD-sadB, E. coli K12 ΔpfkA ΔpfkB ΔfbaA ΔfbaB ΔsthA Δgnd pCL1925-edp3 pTrc99A::budB-ilvC-ilvD-kivD-sadB, E. coli K12 ΔpfkA ΔpfkB ΔfbaA ΔfbaB ΔsthA Δgnd pCL1925-edp4 pTrc99A::budB-ilvC-ilvD-kivD-sadB, E. coli K12 ΔpfkA ΔpfkB ΔfbaA ΔfbaB ΔsthA Δgnd pCL1925-edp1-edp2 pTrc99A::budB-ilvC-ilvD-kivD-sadB, E. coli K12 ΔpfkA ΔpfkB ΔfbaA ΔfbaB ΔsthA Δgnd pCL1925-edp1-edp3 pTrc99A::budB-ilvC-ilvD-kivD-sadB, E. coli K12 ΔpfkA ΔpfkB ΔfbaA ΔfbaB ΔsthA Δgnd pCL1925-edp1-edp4 pTrc99A::budB-ilvC-ilvD-kivD-sadB, E. coli K12 ΔpfkA ΔpfkB ΔfbaA ΔfbaB ΔsthA Δgnd pCL1925-edp1-edp2-edp3 pTrc99A::budB-ilvC-ilvD-kivD-sadB, E. coli K12 ΔpfkA ΔpfkB ΔfbaA ΔfbaB ΔsthA Δgnd pCL1925-edp1-edp2-edp4 pTrc99A::budB-ilvC-ilvD-kivD-sadB, E. coli K12 ΔpfkA ΔpfkB ΔfbaA ΔfbaB ΔsthA Δgnd pCL1925-edp1-edp2-edp3-edp4 pTrc99A::budB-ilvC-ilvD-kivD-sadB, E. coli K12 Δpgi pTrc99A::budB-ilvC-ilvD-kivD-sadB, E. coli K12 Δpgi ΔsthA pTrc99A::budB-ilvC-ilvD-kivD-sadB, E. coli K12 Δpgi Δgnd pTrc99A::budB-ilvC-ilvD-kivD-sadB, E. coli K12 Δpgi pCL1925-edp1 pTrc99A::budB-ilvC-ilvD-kivD-sadB, E. coli K12 Δpgi pCL1925-edp2 pTrc99A::budB-ilvC-ilvD-kivD-sadB, E. coli K12 Δpgi pCL1925-edp3 pTrc99A::budB-ilvC-ilvD-kivD-sadB, E. coli K12 Δpgi pCL1925-edp4 pTrc99A::budB-ilvC-ilvD-kivD-sadB, E. coli K12 Δpgi ΔsthA Δgnd pTrc99A::budB-ilvC-ilvD-kivD-sadB, E. coli K12 Δpgi ΔsthA pCL1925-edp1 pTrc99A::budB-ilvC-ilvD-kivD-sadB, E. coli K12 Δpgi ΔsthA pCL1925-edp2 pTrc99A::budB-ilvC-ilvD-kivD-sadB, E. coli K12 Δpgi ΔsthA pCL1925-edp3 pTrc99A::budB-ilvC-ilvD-kivD-sadB, E. coli K12 Δpgi ΔsthA pCL1925-edp4 pTrc99A::budB-ilvC-ilvD-kivD-sadB, E. coli K12 Δpgi ΔsthA Δgnd pCL1925-edp1 pTrc99A::budB-ilvC-ilvD-kivD-sadB, E. coli K12 Δpgi ΔsthA Δgnd pCL1925-edp2 pTrc99A::budB-ilvC-ilvD-kivD-sadB, E. coli K12 Δpgi ΔsthA Δgnd pCL1925-edp3 pTrc99A::budB-ilvC-ilvD-kivD-sadB, E. coli K12 Δpgi ΔsthA Δgnd pCL1925-edp4 pTrc99A::budB-ilvC-ilvD-kivD-sadB, E. coli K12 Δpgi ΔsthA Δgnd pCL1925-edp1-edp2 pTrc99A::budB-ilvC-ilvD-kivD-sadB, E. coli K12 Δpgi ΔsthA Δgnd pCL1925-edp1-edp3 pTrc99A::budB-ilvC-ilvD-kivD-sadB, E. coli K12 Δpgi ΔsthA Δgnd pCL1925-edp1-edp4 pTrc99A::budB-ilvC-ilvD-kivD-sadB, E. coli K12 Δpgi ΔsthA Δgnd pCL1925-edp1-edp2-edp3 pTrc99A::budB-ilvC-ilvD-kivD-sadB, E. coli K12 Δpgi ΔsthA Δgnd pCL1925-edp1-edp2-edp4 pTrc99A::budB-ilvC-ilvD-kivD-sadB, E. coli K12 Δpgi ΔsthA Δgnd pCL1925-edp1-edp2-edp3-edp4 pTrc99A::budB-ilvC-ilvD-kivD-sadB, E. coli K12 Δzwf pCL1925-edp1 pTrc99A::budB-ilvC-ilvD-kivD-sadB, E. coli K12 Δzwf ΔpfkA pCL1925-edp1 pTrc99A::budB-ilvC-ilvD-kivD-sadB, E. coli K12 Δzwf ΔpfkA ΔpfkB pCL1925-edp1 pTrc99A::budB-ilvC-ilvD-kivD-sadB, E. coli K12 Δzwf ΔpfkA ΔpfkB ΔfbaA pCL1925-edp1 pTrc99A::budB-ilvC-ilvD-kivD-sadB, E. coli K12 Δzwf ΔpfkA ΔpfkB ΔfbaA ΔfbaB pCL1925-edp1 pTrc99A::budB-ilvC-ilvD-kivD-sadB, E. coli K12 Δzwf ΔpfkA ΔpfkB ΔfbaA ΔfbaB ΔsthA pCL1925-edp1 pTrc99A::budB-ilvC-ilvD-kivD-sadB, E. coli K12 Δzwf ΔpfkA ΔpfkB ΔfbaA ΔfbaB ΔsthA Δgnd pCL1925-edp1 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp2 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp3 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp4 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp2-edp3 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp2-edp4 pTrc99A::budB-ilvC-ilvD-kivD-sadB, E. coli K12 Δzwf ΔpfkA ΔpfkB ΔfbaA ΔfbaB ΔsthA Δgnd pCL1925-edp1-edp2-edp3-edp4 pTrc99A::budB-ilvC-ilvD-kivD-sadB, E. coli K12 Δzwf Δpgi pCL1925-edp1 pTrc99A::budB-ilvC-ilvD-kivD-sadB, E. coli K12 Δzwf Δpgi ΔsthA pCL1925-edp1 pTrc99A::budB-ilvC-ilvD-kivD-sadB, E. coli K12 Δzwf Δpgi ΔsthA Δgnd pCL1925-edp1 pTrc99A::budB-ilvC-ilvD-kivD-sadB, E. coli K12 Δzwf Δpgi ΔsthA Δgnd pCL1925-edp1-edp2 pTrc99A::budB-ilvC-ilvD-kivD-sadB, E. coli K12 Δzwf Δpgi ΔsthA Δgnd pCL1925-edp1-edp3 pTrc99A::budB-ilvC-ilvD-kivD-sadB, E. coli K12 Δzwf Δpgi ΔsthA Δgnd pCL1925-edp1-edp4 pTrc99A::budB-ilvC-ilvD-kivD-sadB, E. coli K12 Δzwf Δpgi ΔsthA Δgnd pCL1925-edp1-edp2-edp3 pTrc99A::budB-ilvC-ilvD-kivD-sadB, E. coli K12 Δzwf Δpgi ΔsthA Δgnd pCL1925-edp1-edp2-edp4 pTrc99A::budB-ilvC-ilvD-kivD-sadB, E. coli K12 Δzwf Δpgi ΔsthA Δgnd pCL1925-edp1-edp2-edp3-edp4 pTrc99A::budB-ilvC-ilvD-kivD-sadB.

Construction of a S. cerevisiae Production Host of the Invention

Optimization of isobutanol production by a functional EDP in S. cerevisiae is achieved through following three means: 1) expression and up-regulation of a set of genes that encodes enzymes of an isobutanol production pathway; 2) expression and up-regulation of a set of genes that encodes EDP enzymes; 3) decreasing flux through competing carbon-metabolizing pathways in order to achieve e.g. a diminished EMP and/or a diminished oxidative PPP.

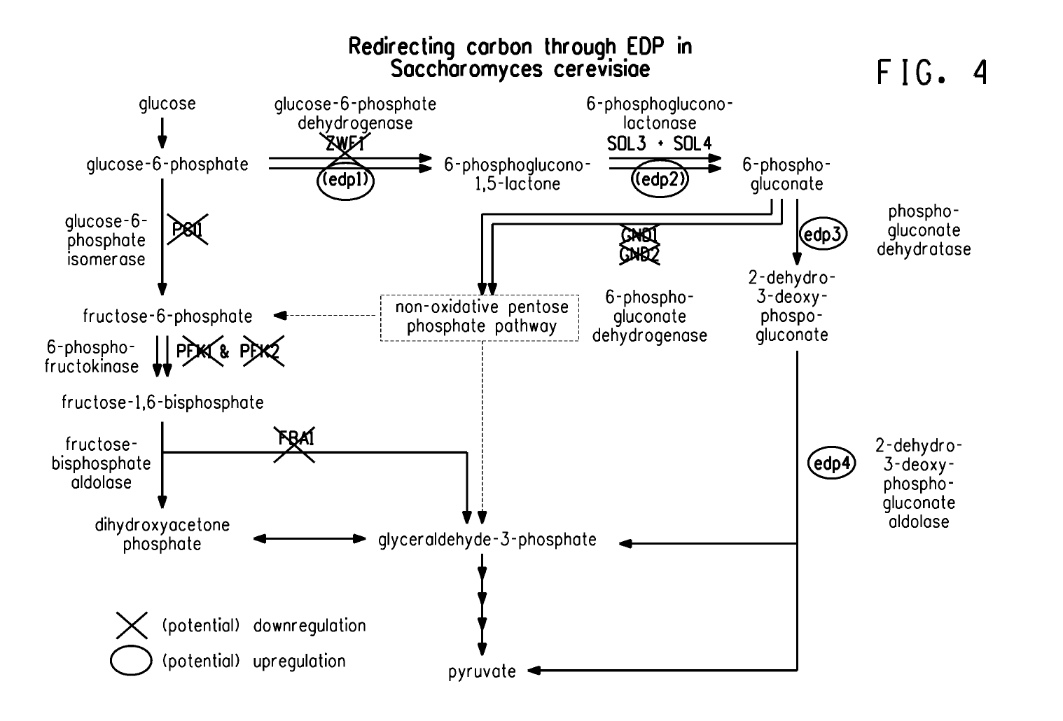

Methods for gene expression in Saccharomyces cerevisiae are known in the art (see for example “Methods in Enzymology”, Volume 194, Guide to Yeast Genetics and Molecular and Cell Biology, (Part A, 2004, Christine Guthrie and Gerald R. Fink (eds.), Elsevier Academic Press, San Diego, Calif.). In brief, expression of genes in yeast typically utilizes a promoter, followed by the gene of interest, and a transcriptional terminator. A number of yeast promoters can be used in constructing expression cassettes for genes encoding an isobutanol biosynthetic pathway, including, but not limited to constitutive promoters FBA, GPD, ADH1, and GPM, and the inducible promoters GAL1, GAL10, and CUP1. Suitable transcriptional terminators include, but are not limited to FBAt, GPDt, GPMt, ERG10t, GAL1t, CYC1, and ADH1. For example, suitable promoters, transcriptional terminators, and the genes of an isobutanol biosynthetic pathway may be cloned into E. coli-yeast shuttle vectors as described in Example 17 of U.S. Patent Application Publication No. 20070092957 which is incorporated by reference herein. Since S. cerevisiae lacks the genes for phophogluconate dehydratase (E.C. 4.2.1.12) and 2-dehydro-3-deoxy-phosphogluconate aldolase (E.C. 4.1.2.14), heterogenous genes that encode phosphogluconate dehydratase (E.C. 4.2.1.12), referred to and afore defined as edp3, and 2-dehydro-3-deoxy-phosphogluconate aldolase (E.C. 4.1.2.14), referred to and afore defined as edp4, can be introduced and expressed in S. cerevisiae (FIG. 4). Additionally, either endogenous genes for glucose-6-phosphate dehydrogenase (E.C. 1.1.1.49) and 6-phosphogluconolactonase (E.C. 3.1.1.31) can be upregulated or activity of their gene products can be augmented and/or replaced by expression and/or upregulation of a heterologues gene or set of genes from the set of genes encoding glucose-6-phosphate dehydrogenase (E.C. 1.1.1.49) and 6-phosphogluconolactonase (E.C. 3.1.1.31), referred to and afore defined as edp1 and edp2 and encoding preferred EDP enzymes, respectively.

Decreasing flux through competing carbon-metabolizing pathways is achieved through the disruption and/or down-regulation of EMP- and PPP-specific genes and their gene products. To diminish oxidative PPP, e.g. the 6-phosphogluconate dehydrogenase activity in S. cerevisiae catalyzed by two isoenzymes encoded by the GND1 (SEQ ID NO: 149, genomic SEQ ID NO: 327) and GND2 (SEQ ID NO: 147, genomic SEQ ID NO: 328) genes, is reduced or completely eliminated by down-regulation and/or deletion of at least one, preferably both of the genes.

To diminish EMP in S. cerevisiae, the gene PGI1 (SEQ ID NO: 158), encoding glucose-6-phosphate isomerase (E.C. 5.3.1.9), is down-regulated or deleted.

Alternatively, flux through 6-phosphofructo-1-kinase reaction (E.C. 2.7.1.11), converting fructose-6-phosphate to fructose-1,6-bisphosphate is reduced or completely eliminated.

In S. cerevisiae, this reaction is catalyzed by two iso-enzymes, encoded by the genes PFK1 (SEQ ID NO: 171, genomic SEQ ID NO: 324) and PFK2 (SEQ ID NO: 173, genomic SEQ ID NO: 325). Diminishing or completely eliminating flux through 6-phosphofructokinase reaction is achieved by either deletion or down-regulation of at least one, preferably both of these genes. Alternatively, rate of the fructose-bisphosphate aldolase reaction (E. C. 4.1.2.13) is diminished or completely eliminated through the down-regulation or deletion of gene FBA1 (SEQ ID NO: 185; genomic SEQ ID NO: 326) in S. cerevisiae. However, reduced or eliminated fructose-bisphosphate aldolase reaction leads to elevated levels of fructose-1,6-bisphosphate in the cells that was found to activate flux through EMP enzymes, but inhibit flux through competing pathways like e.g. oxidative PPP (Kirtley, M. E. et al., supra). Consequently, deletion of 6-phosphofructokinase reaction (E.C. 2.7.1.11), converting fructose-6-phosphate to fructose-1,6-bisphosphate, in conjunction with reduction or complete elimination of the fructose-bisphosphate aldolase reaction (E. C. 4.1.2.13) at the same time is another favorable teaching of the patent to optimize isobutanol production through enhancement of the EDP. In S. cerevisiae, this is achieved by the down-regulation and/or deletion of at least two, preferably more of the gene set comprising PFK1, PFK2 and FBA1. Provided herein is a method of decreasing flux through the competing carbon-metabolizing pathways EMP- and PPP can be achieved through the disruption or down-regulation of one or more genes of the gene set comprising PGI1, PFK1, PFK2, FBA1, GND1 and GND2.

Provided herein are genotypes in S. cerevisiae including: S. cerevisiae pRS411::edp3-edp4 pRS423::CUP1 p-alsS+FBAp-ILV3 pHR81::FBAp-ILV5+GPMp-kivD, S. cerevisiae ΔPFK1 pRS411::edp3-edp4 pRS423::CUP1p-alsS+FBAp-ILV3 pHR81::FBAp-ILV5+GPMp-kivD, S. cerevisiae ΔPFK2 pRS411::edp3-edp4 pRS423::CUP1p-alsS+FBAp-ILV3 pHR81::FBAp-ILV5+GPMp-kivD, S. cerevisiae ΔFBA1 pRS411::edp3-edp4 pRS423::CUP1p-alsS+FBAp-ILV3 pHR81::FBAp-ILV5+GPMp-kivD, S. cerevisiae ΔGND1 pRS411::edp3-edp4 pRS423::CUP1p-alsS+FBAp-ILV3 pHR81::FBAp-ILV5+GPMp-kivD, S. cerevisiae ΔGND2 pRS411::edp3-edp4 pRS423::CUP1p-alsS+FBAp-ILV3 pHR81::FBAp-ILV5+GPMp-kivD, S. cerevisiae pRS411::edp3-edp4-edp1 pRS423::CUP1p-alsS+FBAp-ILV3 pHR81::FBAp-ILV5+GPMp-kivD, S. cerevisiae pRS411::edp3-edp4-edp2 pRS423::CUP1p-alsS+FBAp-ILV3 pHR81::FBAp-ILV5+GPMp-kivD, S. cerevisiae ΔPFK1 ΔPFK2 pRS411::edp3-edp4 pRS423::CUP1p-alsS+FBAp-ILV3 pHR81::FBAp-ILV5+GPMp-kivD, S. cerevisiae ΔPFK1 ΔFBA1 pRS411::edp3-edp4 pRS423::CUP1p-alsS+FBAp-ILV3 pHR81::FBAp-ILV5+GPMp-kivD, S. cerevisiae ΔPFK1 ΔGND1 pRS411::edp3-edp4 pRS423::CUP1p-alsS+FBAp-ILV3 pHR81::FBAp-ILV5+GPMp-kivD, S. cerevisiae ΔPFK1 ΔGND2 pRS411::edp3-edp4 pRS423::CUP1p-alsS+FBAp-ILV3 pHR81::FBAp-ILV5+GPMp-kivD, S. cerevisiae ΔPFK1 pRS411::edp3-edp4-edp1 pRS423::CUP1p-alsS+FBAp-ILV3 pHR81::FBAp-ILV5+GPMp-kivD, S. cerevisiae ΔPFK1 pRS411::edp3-edp4-edp2 pRS423::CUP1p-alsS+FBAp-ILV3 pHR81::FBAp-ILV5+GPMp-kivD, S. cerevisiae ΔPFK1 ΔPFK2 ΔFBA1 pRS411::edp3-edp4 pRS423::CUP1p-alsS+FBAp-ILV3 pHR81::FBAp-ILV5+GPMp-kivD, S. cerevisiae ΔPFK1 ΔPFK2 ΔGND1 pRS411::edp3-edp4 pRS423::CUP1p-alsS+FBAp-ILV3 pHR81::FBAp-ILV5+GPMp-kivD, S. cerevisiae ΔPFK1 ΔPFK2 ΔGND2 pRS411::edp3-edp4 pRS423::CUP1p-alsS+FBAp-ILV3 pHR81::FBAp-ILV5+GPMp-kivD, S. cerevisiae ΔPFK1 ΔPFK2 pRS411::edp3-edp4-edp1 pRS423::CUP1p-alsS+FBAp-ILV3 pHR81::FBAp-ILV5+GPMp-kivD, S. cerevisiae ΔPFK1 ΔPFK2 pRS411::edp3-edp4-edp2 pRS423::CUP1p-alsS+FBAp-ILV3 pHR81::FBAp-ILV5+GPMp-kivD, S. cerevisiae ΔPFK1 ΔPFK2 ΔFBA1 ΔGND1 pRS411::edp3-edp4 pRS423::CUP1p-alsS+FBAp-ILV3 pHR81::FBAp-ILV5+GPMp-kivD, S. cerevisiae ΔPFK1 ΔPFK2 ΔFBA1 ΔGND2 pRS411::edp3-edp4 pRS423::CUP1p-alsS+FBAp-ILV3 pHR81::FBAp-ILV5+GPMp-kivD, S. cerevisiae ΔPFK1 ΔPFK2 ΔFBA1 pRS411::edp3-edp4-edp1 pRS423::CUP1 p-alsS+FBAp-ILV3 pHR81::FBAp-ILV5+GPMp-kivD, S. cerevisiae ΔPFK1 ΔPFK2 ΔFBA1 pRS411::edp3-edp4-edp2 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 ΔGND2 pRS411::edp3-edp4 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 pRS411::edp3-edp4-edp1 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 pRS411::edp3-edp4-edp2 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 ΔGND2 pRS411::edp3-edp4-edp1 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 ΔGND2 pRS411::edp3-edp4-edp2 pRS423::CUP1 p-alsS+FBAp-ILV3 pHR81::FBAp-ILV5+GPMp-kivD, S. cerevisiae ΔPFK1 ΔPFK2 ΔFBA1 ΔGND1 ΔGND2 pRS411::edp3-edp4-edp1-edp2 pRS423::CUP1 p-alsS+FBAp-ILV3 pHR81::FBAp-ILV5+GPMp-kivD, S. cerevisiae ΔPGI1 pRS411::edp3-edp4 pRS423::CUP1p-alsS+FBAp-ILV3 pHR81::FBAp-ILV5+GPMp-kivD, S. cerevisiae ΔGND1 pRS411::edp3-edp4 pRS423::CUP1p-alsS+FBAp-ILV3 pHR81::FBAp-ILV5+GPMp-kivD, S. cerevisiae ΔGND2 pRS411::edp3-edp4 pRS423::CUP1p-alsS+FBAp-ILV3 pHR81::FBAp-ILV5+GPMp-kivD, S. cerevisiae pRS411::edp3-edp4-edp1 pRS423::CUP1p-alsS+FBAp-ILV3 pHR81::FBAp-ILV5+GPMp-kivD, S. cerevisiae pRS411::edp3-edp4-edp2 pRS423::CUP1p-alsS+FBAp-ILV3 pHR81::FBAp-ILV5+GPMp-kivD, S. cerevisiae ΔPGI1 ΔGND1 pRS411::edp3-edp4 pRS423::CUP1p-alsS+FBAp-ILV3 pHR81::FBAp-ILV5+GPMp-kivD, S. cerevisiae ΔPGI1 ΔGND2 pRS411::edp3-edp4 pRS423::CUP1p-alsS+FBAp-ILV3 pHR81::FBAp-ILV5+GPMp-kivD, S. cerevisiae ΔPGI1 pRS411::edp3-edp4-edp1 pRS423::CUP1p-alsS+FBAp-ILV3 pHR81::FBAp-ILV5+GPMp-kivD, S. cerevisiae ΔPGI1 pRS411::edp3-edp-4-edp2 pRS423::CUP1p-alsS+FBAp-ILV3 pHR81::FBAp-ILV5+GPMp-kivD, S. cerevisiae ΔPGI1 ΔGND1 ΔGND2 pRS411::edp3-edp4 pRS423::CUP1p-alsS+FBAp-ILV3 pHR81::FBAp-ILV5+GPMp-kivD, S. cerevisiae ΔPGI1 ΔGND1 pRS411::edp3-edp4-edp1 pRS423::CUP1p-alsS+FBAp-ILV3 pHR81::FBAp-ILV5+GPMp-kivD, S. cerevisiae ΔPGI1 ΔGND1 pRS411::edp3-edp4-edp2 pRS423::CUP1p-alsS+FBAp-ILV3 pHR81::FBAp-ILV5+GPMp-kivD, S. cerevisiae ΔPGI1 ΔGND1 ΔGND2 pRS411::edp3-edp4-edp1 pRS423::CUP1p-alsS+FBAp-ILV3 pHR81::FBAp-ILV5+GPMp-kivD, S. cerevisiae ΔPGI1 ΔGND1 ΔGND2 pRS411::edp3-edp4-edp2 pRS423::CUP1p-alsS+FBAp-ILV3 pHR81::FBAp-ILV5+GPMp-kivD, S. cerevisiae ΔPGI1 ΔGND1 ΔGND2 pRS411::edp3-edp4-edp1-edp2 pRS423::CUP1p-alsS+FBAp-ILV3 pHR81::FBAp-ILV5+GPMp-kivD, S. cerevisiae ΔZWF1 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 ΔPFK2 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 ΔPFK2 ΔFBA1 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 ΔPFK2 ΔFBA1 ΔGND1 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 ΔPFK2 ΔFBA1 ΔGND1 ΔGND2 pRS411::edp3-edp4-edp1 pRS423::CUP1p-alsS+FBAp-ILV3 pHR81::FBAp-ILV5+GPMp-kivD, S. cerevisiae ΔZWF1 ΔPFK1 ΔPFK2 ΔFBA1 ΔGND1 ΔGND2 pRS411::edp3-edp4-edp1-edp2 pRS423::CUP1p-alsS+FBAp-ILV3 pHR81::FBAp-ILV5+GPMp-kivD, S. cerevisiae ΔZWF1 pRS411::edp3-edp4-edp1 pRS423::CUP1p-alsS+FBAp-ILV3 pHR81::FBAp-ILV5+GPMp-kivD, S. cerevisiae ΔZWF1 ΔPGI1 pRS411::edp3-edp4-edp1 pRS423::CUP1p-alsS+FBAp-ILV3 pHR81::FBAp-ILV5+GPMp-kivD, S. cerevisiae ΔZWF1 ΔPGI1 ΔGND1 pRS411::edp3-edp4-edp1 pRS423::CUP1p-alsS+FBAp-ILV3 pHR81::FBAp-ILV5+GPMp-kivD, S. cerevisiae ΔZWF1 ΔPGI1 ΔGND1 ΔGND2 pRS411::edp3-edp4-edp1 pRS423::CUP1p-alsS+FBAp-ILV3 pHR81::FBAp-ILV5+GPMp-kivD, S. cerevisiae ΔZWF1 ΔPGI1 ΔGND1 ΔGND2 pRS411::edp3-edp4-edp1-edp2 pRS423::CUP1p-alsS+FBAp-ILV3 pHR81::FBAp-ILV5+GPMp-kivD.

Construction of a L. plantarum Production Host of the Invention

Optimization of isobutanol production by a functional EDP in L. plantarum can be achieved through the following: 1) expression and up-regulation of a set of genes that encodes enzymes of an isobutanol production pathway; 2) expressing and up-regulating of a set of genes that encodes preferred EDP enzymes; or 3) decreasing flux through competing carbon-metabolizing pathways in order to achieve e.g. a diminished EMP and/or a diminished oxidative PPP. The Lactobacillus genus belongs to the Lactobacillales family and many plasmids and vectors used in the transformation of Bacillus subtilis and Streptococcus may be used for lactobacillus. L. plantarum belongs to the Lactobacillales family and many plasmids and vectors used in the transformation of Bacillus subtilis and Streptococcus may be used for expression and subsequent up-regulation of a set of genes that encodes enzymes of an isobutanol production pathway. Non-limiting examples of suitable vectors include pAMβ1 and derivatives thereof (Renault et al., Gene, 183:175-182 (1996); and O′Sullivan et al., Gene, 137:227-231 (1993)); pMBB1 and pHW800, a derivative of pMBB1 (Wyckoff et al. Appl. Environ. Microbiol., 62:1481-1486, 1996); pMG1, a conjugative plasmid (Tanimoto et al., J. Bacteriol., 184:5800-5804, 2002); pNZ9520 (Kleerebezem et al., Appl. Environ. Microbiol. 63:4581-4584, 1997); pAM401 (Fujimoto et al., Appl. Environ. Microbiol., 67:1262-1267, 2001); and pAT392 (Arthur et al., Antimicrob. Agents Chemother., 38:1899-1903, 1994). Several plasmids from L. plantarum have also been reported (e.g., van Kranenburg R, Golic N, Bongers R, Leer R J, de Vos W M, Siezen R J, Kleerebezem M. Appl. Environ. Microbiol., 71: 1223-1230, 2005). For example, expression of an isobutanol biosynthetic pathway in L. plantarum is described in Example 21 of U.S. Patent Application Publication No. 20070092957 which is incorporated by reference herein. In one embodiment, expression of isobutanol pathway genes is accomplished by, but not limited to, plasmid pDM1-ilvD-ilvC-kivD-sadB-alsS.

Due to the fact that L. plantarum does not contain the genes for phophogluconate dehydratase (E.C. 4.2.1.12) and 2-dehydro-3-deoxy-phosphogluconate aldolase (E.C. 4.1.2.14), heterogenous genes that encode phosphogluconate dehydratase reaction (E.C. 4.2.1.12), referred to and afore defined as edp3, and 2-dehydro-3-deoxy-phosphogluconate aldolase reaction (E.C. 4.1.2.14), referred to and afore defined as edp4, need to be expressed in L. plantarum. Additionally, either endogenous genes for glucose-6-phosphate dehydrogenase (E.C. 1.1.1.49) and/or 6-phosphogluconolactonase (E.C. 3.1.1.31) are up-regulated, or heterogenous genes are expressed and/or up-regulated from the set of genes encoding glucose-6-phosphate dehydrogenase (E.C. 1.1.1.49) and 6-phosphogluconolactonase (E.C. 3.1.1.31), referred to and afore defined as edp1 and edp2 and encoding preferred EDP enzymes, respectively, either to augment or replace activity of the endogenous gene products.

Decreasing flux through competing carbon-metabolizing pathways is achieved through e.g. the disruption or down-regulation of EMP- and PPP-specific genes and their gene products. To diminish oxidative PPP, e.g. the 6-phosphogluconate dehydrogenase activity, in L. plantarum catalyzed by two isoenzymes encoded by the gnd1 (SEQ ID NO: 151) and gnd2 (SEQ ID NO: 153) genes, is reduced or completely eliminated by either down-regulation or deletion of at least one, preferably both of the genes.

To accomplish a diminished EMP in L. plantarum, the gene pgi (SEQ ID NO: 161), encoding glucose-6-phosphate isomerase (E.C. 5.3.1.9), is down-regulated or deleted. Alternatively, flux through 6-phosphofructokinase (E.C. 2.7.1.11), converting fructose-6-phosphate to fructose-1,6-bisphosphate is reduced or completely eliminated. In L. plantarum, an enzyme that catalyzes the reaction is encoded by the gene pfkA (SEQ ID NO: 175). Diminishing or completely eliminating flux through 6-phosphofructokinase reaction is achieved by deletion or down-regulation of the pfkA gene. Alternatively, the rate of the fructosebisphosphate aldolase reaction (E. C. 4.1.2.13) is diminished or completely eliminated through the down-regulation or deletion of gene fba in L. plantarum (SEQ ID NO: 187). However, reduced or eliminated fructose-bisphosphate aldolase reaction leads to elevated levels of fructose-1,6-bisphosphate in the cells that was found to activate flux through EMP enzymes, but inhibit flux through competing pathways like e.g. oxidative PPP. Consequently, deletion of 6-phosphofructokinase reaction (E.C. 2.7.1.11), converting fructose-6-phosphate to fructose-1,6-bisphosphate, in conjunction with reduction or complete elimination of the fructosebisphosphate aldolase reaction (E. C. 4.1.2.13) at the same time is preferred to optimize isobutanol production through enhancement of the EDP. In L. plantarum, this is achieved by the down-regulation and/or deletion of the gene set comprising pfkA (SEQ ID NO: 175) and fba (SEQ ID NO: 187). Provided herein are methods of decreasing flux through the competing carbon-metabolizing pathways EMP- and PPP is achieved through the disruption or down-regulation of one or more genes of the gene set comprising pgi, pfkA, fba, gnd1 and gnd2.

Provided herein are genotypes in L. plantarum including the following: L. plantarum pFP996-edp3-edp4 pDM1-ilvD-ilvC-kivD-sadB-alsS, L. plantarum Δpgi pFP996-edp3-edp4 pDM1-ilvD-ilvC-kivD-sadB-alsS, L. plantarum Δgnd1 Δpgi pFP996-edp3-edp4 pDM1-ilvD-ilvC-kivD-sadB-alsS, L. plantarum Δgnd1 Δgnd2 Δpgi pFP996-edp3-edp4 pDM1-ilvD-ilvC-kivD-sadB-alsS, L. plantarum Δgnd1 Δgnd2 Δpgi pFP996-edp3-edp4-edp1 pDM1-ilvD-ilvC-kivD-sadB-alsS, L. plantarum Δgnd1 Δgnd2 Δpgi pFP996-edp3-edp4-edp2 pDM1-ilvD-ilvC-kivD-sadB-alsS, L. plantarum Δgnd1 Δgnd2 Δpgi pFP996-edp3-edp4-edp1-edp2 pDM1-ilvD-ilvC-kivD-sadB-alsS, L. plantarum Δgnd1 pFP996-edp3-edp4 pDM1-ilvD-ilvC-kivD-sadB-alsS, L. plantarum Δgnd2 pFP996-edp3-edp4 pDM1-ilvD-ilvC-kivD-sadB-alsS, L. plantarum ΔpfkA pFP996-edp3-edp4 pDM1-ilvD-ilvC-kivD-sadB-alsS, L. plantarum Δfba pFP996-edp3-edp4 pDM1-ilvD-ilvC-kivD-sadB-alsS, L. plantarum pFP996-edp3-edp4-edp1 pDM1-ilvD-ilvC-kivD-sadB-alsS, L. plantarum pFP996-edp3-edp4-edp2 pDM1-ilvD-ilvC-kivD-sadB-alsS, L. plantarum Δgnd1 Δgnd2 pFP996-edp3-edp4 pDM1-ilvD-ilvC-kivD-sadB-alsS, L. plantarum Δgnd1 ΔpfkA pFP996-edp3-edp4 pDM1-ilvD-ilvC-kivD-sadB-alsS, L. plantarum Δgnd1 Δfba pFP996-edp3-edp4 pDM1-ilvD-ilvC-kivD-sadB-alsS, L. plantarum Δgnd1 pFP996-edp3-edp4-edp1 pDM1-ilvD-ilvC-kivD-sadB-alsS, L. plantarum Δgnd1 pFP996-edp3-edp4-edp2 pDM1-ilvD-ilvC-kivD-sadB-alsS, L. plantarum Δgnd1 Δgnd2 ΔpfkA pFP996-edp3-edp4 pDM1-ilvD-ilvC-kivD-sadB-alsS, L. plantarum Δgnd1 Δgnd2 Δfba pFP996-edp3-edp4 pDM1-ilvD-ilvC-kivD-sadB-alsS, L. plantarum Δgnd1 Δgnd2 pFP996-edp3-edp4-edp1 pDM1-ilvD-ilvC-kivD-sadB-alsS, L. plantarum Δgnd1 Δgnd2 pFP996-edp3-edp4-edp2 pDM1-ilvD-ilvC-kivD-sadB-alsS, L. plantarum Δgnd1 Δgnd2 ΔpfkA Δfba pFP996-edp3-edp4 pDM1-ilvD-ilvC-kivD-sadB-alsS, L. plantarum Δgnd1 Δgnd2 ΔpfkA pFP996-edp3-edp4-edp1 pDM1-ilvD-ilvC-kivD-sadB-alsS, L. plantarum Δgnd1 Δgnd2 ΔpfkA pFP996-edp3-edp-4-edp2 pDM1-ilvD-ilvC-kivD-sadB-alsS, L. plantarum Δgnd1 Δgnd2 ΔpfkA Δfba pFP996-edp3-edp4-edp1 pDM1-ilvD-ilvC-kivD-sadB-alsS, L. plantarum Δgnd1 Δgnd2 ΔpfkA Δfba pFP996-edp3-edp-4-edp2 pDM1-ilvD-ilvC-kivD-sadB-alsS, L. plantarum Δgnd1 Δgnd2 ΔpfkA Δfba pFP996-edp3-edp4-edp1-edp2 pDM1-ilvD-ilvC-kivD-sadB-alsS, L. plantarum Δgpd Δgnd1 Δgnd2 Δpgi pFP996-edp3-edp4-edp1 pDM1-ilvD-ilvC-kivD-sadB-alsS, L. plantarum Δgpd Δgnd1 Δgnd2 Δpgi pFP996-edp3-edp4-edp1-edp2 pDM1-ilvD-ilvC-kivD-sadB-alsS, L. plantarum Δgpd pFP996-edp3-edp4-edp1 pDM1-ilvD-ilvC-kivD-sadB-alsS, L. plantarum Δgpd Δgnd1 pFP996-edp3-edp4-edp1 pDM1-ilvD-ilvC-kivD-sadB-alsS, L. plantarum Δgpd Δgnd1 Δgnd2 pFP996-edp3-edp4-edp1 pDM1-ilvD-ilvC-kivD-sadB-alsS, L. plantarum Δgpd Δgnd1 Δgnd2 ΔpfkA pFP996-edp3-edp4-edp1 pDM1-ilvD-ilvC-kivD-sadB-alsS, L. plantarum Δgpd Δgnd1 Δgnd2 ΔpfkA Δfba pFP996-edp3-edp4-edp1 pDM1-ilvD-ilvC-kivD-sadB-alsS, L. plantarum Δgpd Δgnd1 Δgnd2 ΔpfkA Δfba pFP996-edp3-edp4-edp1-edp2 pDM1-ilvD-ilvC-kivD-sadB-alsS.

### Carbohydrate Metabolism and Carbon Substrates

Glucose- or fructose-derivatives, like e.g. glucose-1-phosphate, glucose-6-phosphate, fructose-1-phosphate or fructose-6-phosphate, are central and typically interconvertable metabolites in most of the common carbohydrate-metabolizing pathways and their substrates, including, but not limited to, monosaccharides such as glucose and fructose, oligosaccharides such as lactose or sucrose, polysaccharides/glucans such as starch or cellulose or mixtures thereof and unpurified mixtures from renewable feedstocks such as cheese whey permeate, cornsteep liquor, sugar beet molasses, and barley malt.

Recombinant bacteria or yeast hosts disclosed herein are contacted with fermentation media which contains suitable carbon substrates for isobutanol production. Suitable carbon substrates may include but are not limited to monosaccharides such as glucose and fructose, oligosaccharides such as lactose, maltose, galactose, or sucrose, polysaccharides/glucans such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol.

Methylotrophic organisms are also known to utilize a number of other carbon containing compounds such as methylamine, glucosamine and a variety of amino acids for metabolic activity. For example, methylotrophic yeasts are known to utilize the carbon from methylamine to form trehalose or glycerol (Bellion et al., Microb. Growth C1 Compd., [Int. Symp.], 7th (1993), 415-32, Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in the present invention may encompass a wide variety of carbon containing substrates and will only be limited by the choice of organism.

Although it is contemplated that all of the mentioned carbon substrates and mixtures thereof are suitable in the present invention, preferred carbon substrates are glucose, fructose, and sucrose, or mixtures of these with C5 sugars such as xylose and/or arabinose for yeasts cells modified to use C5 sugars. Sucrose may be derived from renewable sugar sources such as sugar cane, sugar beets, cassava, sweet sorghum, and mixtures thereof. Glucose and dextrose may be derived from renewable grain sources through saccharification of starch based feedstocks including grains such as corn, wheat, rye, barley, oats, and mixtures thereof. In addition, fermentable sugars may be derived from renewable cellulosic or lignocellulosic biomass (including hemicellulose) through processes of pretreatment and saccharification, as described, for example, in U.S. Patent Application Publication No. 20070031918A1, which is herein incorporated by reference. Biomass may include both five carbon (e.g., xylose, arabinose) and six carbon sugars. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass may comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, animal manure, and mixtures thereof.

In addition to an appropriate carbon source, fermentation media must contain suitable minerals, salts, cofactors, buffers and other components, known to those skilled in the art, suitable for the growth of the cultures and promotion of the enzymatic pathway necessary for isobutanol production.

### Determination of Flux

While not wishing to be bound by theory, it is believed that modification of the EDP, PPP, or EMP of a host cell as provided herein will provide increased flux through the EDP, and consequently will provide optimized production and utilization of reducing equivalents for isobutanol production. Enhanced EDP can be confirmed using ¹³C tracer analysis methodology known in the art and exemplified herein (see prophetic Example 17). In preferred embodiments, the microbial host cell comprises an enhanced EDP and an increased relative flux through the EDP under anaerobic conditions. In preferred embodiments, the relative flux through at least one reaction unique to the EDP under anaerobic conditions is at least 1% greater than that in the control host, demonstrating that isobutanol is produced with the help of a functional and/or enhanced ED pathway. In other preferred embodiments, the relative flux through at least one reaction unique to the EDP is at least about 10%, 50%, or 90% greater than that in the control host. In other embodiments, the relative flux through a reaction unique to the EMP or PPP is at least 1% less than that in the control host, demonstrating that isobutanol is produced with the help of a functional and/or enhanced EDP pathway. In preferred embodiments, microbial host cells comprise an increase in relative flux through the EDP with a concomitant decrease in the EMP and PPP.

### Aerobic and Anaerobic Conditions

While it is contemplated that microbial host cells provided herein are suitable for isobutanol production under aerobic conditions, it is believed that microbial host cells provided herein which produce isobutanol are particularly suitable for isobutanol production under anaerobic conditions because the production and subsequent utilization of reducing equivalents is optimized. Therefore, particularly preferred embodiments include microbial host cells comprising an enhanced EDP and/or a diminished EMP and/or PPP and which produce isobutanol under anaerobic conditions. Provided herein are methods of producing isobutanol comprising providing a microbial host cell disclosed herein and contacting the host cell with a fermentable carbon substrate under anaerobic conditions.

### Cofactor Preference

Although the descriptions of isobutanol pathways provided herein assume particular cofactor production and utilization specificities, it is also understood that useful enzymes with different preferences may be identified, engineered, and employed. For example, a KARI enzyme which utilizes NADH has been described in U.S. Patent Application Publication No. US20090163376, and may be employed in an isobutanol production pathway. It is contemplated herein that the EDP, EMP, and/or PPP can likewise be modified such that the cofactor specificity is coordinated. Thus, in one embodiment, provided herein are recombinant microbial host cells which produce isobutanol and comprise an alteration in the EDP, EMP, or PPP such that the reducing equivalents produced during the conversion of glucose to pyruvate are matched with the cofactors required for the conversion of pyruvate to isobutanol. In another embodiment, provided herein are methods of isobutanol production comprising altering the EDP, EMP, or PPP of a microbial host cell such that the reducing equivalents produced during the conversion of glucose to pyruvate are matched with the cofactors required for the conversion of pyruvate to isobutanol.

### Culture Conditions

Typically cells are grown at a temperature in the range of about 25° C. to about 40° C. in an appropriate medium. Suitable growth media in the present invention are common commercially prepared media such as Luria Bertani (LB) broth. Other defined or synthetic growth media may also be used, and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium.

Suitable pH ranges for the fermentation of yeast are typically between pH 3.0 to pH 9.0, where pH 5.0 to pH 8.0 is preferred as the initial condition. Suitable pH ranges for the fermentation of other microorganisms are between pH 3.0 to pH7.5, where pH 4.5.0 to pH 6.5 is preferred as the initial condition.

Production of isobutanol may be performed under aerobic or anaerobic conditions, where anaerobic or microaerobic conditions are preferred.

The amount of isobutanol produced in the fermentation medium can be determined using a number of methods known in the art, for example, high performance liquid chromatography (HPLC) or gas chromatography (GC).

### Batch and Continuous Fermentations

A batch method of fermentation may be used. A classical batch fermentation is a closed system where the composition of the medium is set at the beginning of the fermentation and not subject to artificial alterations during the fermentation. Thus, at the beginning of the fermentation the medium is inoculated with the desired organism or organisms, and fermentation is permitted to occur without adding anything to the system. Typically, however, a “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to an exponential phase and finally to a stationary phase where growth rate is diminished or halted. If untreated, cells in the stationary phase will eventually die. Cells in log phase generally are responsible for the bulk of production of end product or intermediate.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the media. Measurement of the actual substrate concentration in fed-batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Thomas D. Brock in (Biotechnology: A Textbook of Industrial Microbiology, Second Edition, 1989, Sinauer Associates, Inc., Sunderland, Mass.), or in Deshpande, Mukund V., (Appl. Biochem. Biotechnol., 36:227, 1992), herein incorporated by reference.

Although the present invention is performed in batch mode it is contemplated that the method would be adaptable to continuous fermentation methods. Continuous fermentation is an open system where a defined fermentation medium is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to the medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present invention may be practiced using either batch, fed-batch or continuous processes and that any known mode of fermentation would be suitable. Additionally, it is contemplated that cells may be immobilized on a substrate as whole cell catalysts and subjected to fermentation conditions for isobutanol production.

Methods for Isobutanol Isolation from the Fermentation Medium

The bioproduced isobutanol may be isolated from the fermentation medium using methods known in the art. For example, solids may be removed from the fermentation medium by centrifugation, filtration, decantation, or the like. Then, the isobutanol may be isolated from the fermentation medium, which has been treated to remove solids as described above, using methods such as distillation, liquid-liquid extraction, or membrane-based separation. Because isobutanol forms a low boiling point, azeotropic mixture with water, distillation can only be used to separate the mixture up to its azeotropic composition. Distillation may be used in combination with another separation method to obtain separation around the azeotrope. Methods that may be used in combination with distillation to isolate and purify isobutanol include, but are not limited to, decantation, liquid-liquid extraction, adsorption, and membrane-based techniques. Additionally, isobutanol may be isolated using azeotropic distillation using an entrainer (see for example Doherty and Malone, Conceptual Design of Distillation Systems, McGraw Hill, N.Y., 2001).

The isobutanol-water mixture forms a heterogeneous azeotrope so that distillation may be used in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase may be returned to the first distillation column as reflux. The isobutanol-rich decanted organic phase may be further purified by distillation in a second distillation column.

The isobutanol may also be isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the isobutanol from the solvent.

Distillation in combination with adsorption may also be used to isolate isobutanol from the fermentation medium. In this method, the fermentation broth containing the isobutanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245: 199-210, 2004).

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “hr” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “pmole” means micromole(s), “g” means gram(s), “μg” means microgram(s), “mg” means milligram(s), “g” means the gravitation constant, “rpm” means revolutions per minute, “U/mg protein” means unit per milligram of protein, “μg/mL” means microgram per milliliter, “kb” means kilobase, “id” means internal diameter, “° C./min” means degress Celsius per minute, “mL/min” means milliliter per minute, “Ω” means ohm, “sec” means second(s), “min” means minute(s), “μF” means micro Faraday.

### General Methods:

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987.

Materials and methods suitable for the maintenance and growth of bacterial cultures are also well known in the art. Techniques suitable for use in the following Examples may be found, for example, in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

Microbial strains were obtained from The American Type Culture Collection (ATCC), Manassas, Va., unless otherwise noted.

Gene deletions in E. coli can be carried out by standard molecular biology techniques appreciated by one skilled in the art. For example, to create an E. coli strain deleted in a particular gene activity, the gene is deleted by replacing it with an antibiotic resistance marker using the Lambda Red-mediated homologous recombination system as described by Datsenko and Wanner (Proc. Natl. Acad. Sci. USA, 97: 6640-6645, 2000). The Keio collection of E. coli strains (Baba et al., Mol. Syst. Biol., 2:1-11, 2006) is a library of single gene knockouts created in strain E. coli BW25113 by the method of Datsenko and Wanner (supra). In the collection, each deleted gene was replaced with a FRT-flanked kanamycin marker that was removable by Flp recombinase. Alternatively an antibiotic marker may be flanked by other site-specific recombination sequences such as loxP removable by the bacteriophage P1 Cre recombinase (Hoess, R. H. & Abremski, K., J Mol. Biol., 181:351-362, 1985).

### P1 Transduction

P1vir transductions were carried out as described by Miller with some modifications (Miller, J. H. 1992. A Short Course in Bacterial Genetics. Cold Spring Harbor Laboratory, Cold Spring Harbor, N.Y.). Briefly, to prepare a transducing lysate, cells of the donor strain were grown overnight in the Luria Broth (LB) medium at 37 C while shaking. An overnight growth of these cells was sub-cultured into the LB medium containing 0.005M CaCl₂ and placed in a 37 C water bath with no aeration. One hour prior to adding phage, the cells were placed at 37 C with shaking. After final growth of the cells, a 1.0 mL aliquot of the culture was dispensed into 14-ml Falcon tubes and approximately 10e7 P1vir phage/mL was added. These tubes were incubated in a 37 C water bath for 20 min before 2.5 mL of 0.8% LB top agar was added to each tube, the contents were spread on an LB agar plate and were incubated at 37 C. The following day the soft agar layer was scraped into a centrifuge tube. The surface of the plate was washed with the LB medium and added to the centrifuge tube followed by a few drops of CHCl₃ before the tube was vigorously agitated using a Vortex mixer. After centrifugation at 4,000 rpm for 10 min, the supernatant containing the P1vir lysate was collected.

For transduction, the recipient strain was grown overnight in 1-2 mL of the LB medium at 37 C with shaking. Cultures were pelleted by centrifugation in an Eppendorf Microcentrifuge at 10,000 rpm for 1 min at room temp. The cell pellet was resuspended in an equal volume of MC buffer (0.1 M MgSO4, 0.005 M CaCl₂), dispensed into tubes in 0.1 mL aliquots and 0.1 ml and 0.01 ml of P1vir lysate was added. A control tube containing no P1vir lysate was also included. Tubes were incubated for 20 min at 37 C before 0.2 mL of 0.1 M sodium citrate was added to stop the P1 infection. One mL of the LB medium was added to each tube before they were incubated at 37 C for 1 hr. After incubation the cells were pelleted as described above, resuspended in 50-200 μl of the LB prior to spreading on the LB plates containing 25 μg/mL kanamycin and were incubated overnight at 37 C Transductants were screened by colony PCR with chromosome specific primers flanking the region upstream and downstream of the kanamycin marker insertion.

### Marker Removal

Removal of the FRT-flanked kanamycin marker from the chromosome was obtained by transforming the kanamycin-resistant strain with plasmid pCP20 (Cherepanov, P. P. and Wackernagel, W., Gene, 158: 9-14, 1995; available from The Coli Genetic Stock Center at Yale, Cat. No. 7629) followed by spreading onto the LB ampicillin (100 μg/mL) plates and incubating at 30 C. The pCP20 plasmid carries the yeast FLP recombinase under the control of the γ PR promoter. Expression from this promoter is controlled by the cl857 temperature-sensitive repressor residing on the plasmid. The origin of replication of pCP20 is also temperature-sensitive. Ampicillin resistant colonies were streaked onto the LB agar plates and incubated at 42 C. The higher incubation temperature simultaneously induced expression of the FLP recombinase and cured the pCP20 plasmid from the cell. Isolated colonies were patched to grids onto the LB plates containing kanamycin (25 μg/mL), and LB ampicillin (100 μg/mL) plates and LB plates. The resulting kanamycin-sensitive, ampicillin-sensitive colonies were screened by colony PCR to confirm removal of the kanamycin marker from the chromosome.

Removal of the loxP-flanked kanamycin marker from the chromosome was performed by transforming the kanamycin-resistant strain with pJW168 an ampicillin-resistant plasmid (Wild et al., Gene. 223:55-66, 1998) harboring the bacteriophage P1 Cre recombinase. Cre recombinase (Hoess, R. H. & Abremski, K., supra) meditates excision of the kanamycin resistance gene via recombination at the loxP sites. Transformants are spread on LB ampicillin (100 μg/mL) plates and incubated at 30 C. Ampicillin resistant colonies were streaked onto the LB agar plates and incubated at 42 C. The higher incubation temperature cured the temperature-sensitive pJW168 plasmid from the cell. Isolated colonies were patched to grids onto the LB plates containing kanamycin (25 μg/mL), and LB ampicillin (100 μg/mL) plates and LB plates. The resulting kanamycin-sensitive, ampicillin-sensitive colonies were screened by colony PCR to confirm removal of the kanamycin marker from the chromosome.

For colony PCR amplifications the HotStarTaq Master Mix (Qiagen, Valencia, Calif.; catalog no. 71805-3) was used according to the manufacturer's protocol. Into a 25 μL Master Mix reaction containing 0.2 μM of each chromosome specific PCR primer, a small amount of a colony was added. Amplification was carried out in a DNA Thermocycler GeneAmp 9700 (PE Applied Biosystems, Foster City, Calif.). Typical colony PCR conditions were: 15 min at 95° C.; 30 cycles of 95° C. for 30 sec, annealing temperature ranging from 50-58° C. for 30 sec, primers extended at 72° C. with an extension time of approximately 1 min/kb of DNA; then 10 min at 72° C. followed by a hold at 4oC. PCR product sizes were determined by gel electrophoresis by comparison with known molecular weight standards.

Restriction enzymes, T4 DNA ligase and Phusion High Fidelity DNA Polymerase (New England Biolabs, Beverely, Mass.) were used according to manufacturer's recommendation.

Plasmid DNA was prepared using the QIAprep Spin Miniprep kit (Qiagen, Valencia, Calif.; catalog no. 27106) according to manufacturer's recommendations. DNA fragments were extracted from gels using the Zymoclean Gel Extraction Kit (Zymo Research Corp. Orange, Calif.) Gel electrophoresis used the RunOne electrophoresis system (Embi Tec, San Diego, Calif.) with precast Reliant® 1% agarose gels (Lonza Rockland, Inc. Rockland, Me.) according to manufacturer's protocols. Gels are typically run in TBE buffer (Invitrogen, Cat. No. 15581-044).

For transformations, electrocompetent cells of E. coli were prepared as described by Ausubel, F. M., et al., (Current Protocols in Molecular Biology, 1987, Wiley-Interscience,). Cells were grown in 25-50 ml the LB medium at 30-37° C. and harvested at anOD600 of 0.5-0.7 by centrifugation at 10,000 rpm for 10 minutes. These cells are washed twice in sterile ice-cold water in a volume equal to the original starting volume of the culture. After the final wash cells were resuspended in sterile water and the DNA to be transformed was added. The cells and DNA were transferred to chilled cuvettes and electroporated in a Bio-Rad Gene Pulser II according to manufacturer's instructions (Bio-Rad Laboratories, Inc Hercules, Calif.).

The oligonucleotide primers to use in the following Examples are given in Table 5. All the oligonucleotide primers were synthesized by Integrated DNA Technologies, Inc. (Coralville, Iowa).

### Methods for Determining Isobutanol Concentration in the Culture Medium

The concentration of isobutanol in the medium can be determined by a number of methods known in the art. For example, a specific high performance liquid chromatography (HPLC) method using a Shodex SH-1011 column with a Shodex SH-G guard column, (Waters Corporation, Milford, Mass.), with refractive index (RI) detection may be used. Chromatographic separation can be achieved using 0.01 M H₂SO₄ as the mobile phase with a flow rate of 0.5 mL/min and a column temperature of 50° C. Isobutanol has a retention time of 46.6 min under these conditions. Alternatively, gas chromatography (GC) methods are available. For example, isobutanol can be detected using an HP-INNOWax GC column (30 m×0.53 mm id, 1 μm film thickness, Agilent Technologies, Wilmington, Del.), with a flame ionization detector (FID) using the following method: The carrier gas helium at a flow rate of 4.5 mL/min, at 150° C. with constant head pressure; injector split of 1:25 at 200° C.; oven temperature of 45° C. for 1 min, 45 to 220° C. at 10° C./min, and 220° C. for 5 min; and FID detection at 240° C. with 26 mL/min helium makeup gas. The retention time of isobutanol under these conditions is 4.5 min.

### Examples

### Example 1

### Prophetic

### Deletion of 6-phosphogluconate Dehydrogenase Genes in E. coli

Gene deletions in E. coli can be carried out by standard molecular biology techniques appreciated by one skilled in the art. To create an E. coli strain Δgnd in E. coli K12 MG1655, the gene is deleted by replacing it with a kanamycin resistance marker using the Lambda Red-mediated homologous recombination system as described by Datsenko and Wanner (Proc. Natl. Acad. Sci. USA, 97: 6640-6645, 2000). PCR amplification with pKD13 (Datsenko and Wanner, supra) as template and primers GND H1 (SEQ ID NO: 227) and GND H2 (SEQ ID NO: 228) produces a 1.4 kb product. Primer GND H1 consists of the first 50 by of the CDS of gnd followed by 20 nucleotides homologous to the P1 site of pKD13. The GND H2 primer consists of the last 50 base pairs of the gnd CDS followed by 20 bps homologous to the P2 sequence of pKD13. PCR amplification uses the HotStarTaq Master Mix (Qiagen, Valencia, Calif.; catalog no. 71805-3) according to the manufacturer's protocol. Amplification is carried out in a DNA Thermocycler GeneAmp 9700 (PE Applied Biosystems, Foster City, Calif.). The PCR product is gel-purified from a 1% agarose gel with a Qiaquick Gel Extraction Kit (Qiagen Inc., Valencia, Calif.).

E. coli MG1655 harboring pKD46, the temperature sensitive Red recombinase plasmid (Datsenko and Wanner, supra), is grown in 50 mL LB medium with 100 μg/mL ampicillin and 20 mM L-arabinose at 30° C. to an OD600 of 0.5-0.7. Electrocompetent cells of E. coli MG1655/pKD46 are then prepared as described by Ausubel, F. M., et al., (Current Protocols in Molecular Biology, 1987, Wiley-Interscience,). E. coli MG1655/pKD46 is electrotransformed with up to 1 μg of the 1.4 kb PCR product in a Bio-Rad Gene Pulser II according to manufacturer's instructions (Bio-Rad Laboratories Inc, Hercules, Calif.). After electroporation cells are outgrown in SOC medium (2% Bacto Tryptone (Difco), 0.5% yeast extract (Difco), 10 mM NaCl, 2.5 mM KCL, 10 mM MgCl₂, 10 mM MgSO₄, 20 mM glucose) for 2 hours at 30° C. with shaking. Transformants are spread onto LB plates containing kanamycin (25 μg/mL) and incubated overnight at 37° C. to cure the temperature sensitive recombinase plasmid.

Transformants are patched to grids onto LB plates containing kanamycin (25 μg/mL), and LB ampicillin (100 μg/mL) to test for loss of the ampicillin resistant recombinase plasmid, pKD46. Ampillicin-sensitive kanamycin resistant transformants are further analyzed by colony PCR using primers GND Ck UP (SEQ ID NO: 229) and GND Ck Dn (SEQ ID NO: 230), for the expected 1.6 kb PCR fragment. For colony PCR amplifications the HotStarTaq Master Mix (Qiagen, Valencia, Calif.; catalog no. 71805-3) is used according to the manufacturer's protocol. Amplification is carried out in a DNA Thermocycler GeneAmp 9700 (PE Applied Biosystems, Foster City, Calif.). PCR product sizes are determined by gel electrophoresis by comparison with known molecular weight standards. This way strain E. coli K12 MG1655 Δgnd is obtained and validated to be E. coli K12 MG1655 Δgnd.

### Example 2

### Prophetic

### Expression of Isobutanol Production Pathway in E. coli

Expression of heterologous genes encoding an isobutanol production pathway in an E. coli gene deletion strain can be carried out by standard molecular biology techniques that can be appreciated by one skilled in the art. A DNA fragment encoding a butanol dehydrogenase (DNA SEQ ID NO:103; protein SEQ ID NO: 104) from Achromobacter xylosoxidans is amplified from A. xylosoxidans genomic DNA using standard conditions. The DNA is prepared using a Gentra Puregene kit (Gentra Systems, Inc., Minneapolis, Minn.; catalog number D-5500A) following the recommended protocol for gram negative organisms. PCR amplification is done using forward and reverse primers N473 and N469 (SEQ ID NOs: 231 and 232), respectively with Phusion high Fidelity DNA Polymerase (New England Biolabs, Beverly, Mass.). The PCR product is TOPO-Blunt cloned into pCR4 BLUNT (Invitrogen) to produce pCR4Blunt::sadB, which is transformed into E. coli Mach-1 cells. Plasmid is subsequently isolated from an obtained clone, and the sequence verified. The sadB coding region is then cloned into the vector pTrc99a (Amann et al., Gene 69: 301-315, 1988). The pCR4Blunt::sadB is digested with EcoRI, releasing the sadB fragment, which is ligated with EcoRI-digested pTrc99a to generate pTrc99a::sadB. This plasmid is transformed into E. coli Mach 1 cells and the resulting transformant is named Mach1/pTrc99a::sadB. The sadB gene is then subcloned into pTrc99A::budB-ilvC-ilvD-kivD as described below. The pTrc99A::budB-ilvC-ilvD-kivD is the pTrc-99a expression vector carrying an operon for isobutanol expression (described in Examples 9-14 of the U.S. Patent Application Publication No. 20070092957, which are incorporated herein by reference). The first gene in the pTrc99A::budB-ilvC-ilvD-kivD isobutanol operon is budB encoding acetolactate synthase from Klebsiella pneumoniae ATCC 25955, followed by the ilvC gene encoding acetohydroxy acid reductoisomerase from E. coli. This is followed by ilvD encoding acetohydroxy acid dehydratase from E. coli and lastly the kivD gene encoding the branched-chain keto acid decarboxylase from L. lactis. The sadB coding region is amplified from pTrc99a::sadB using primers N695A (SEQ ID NO: 233) and N696A (SEQ ID NO: 234) with Phusion High Fidelity DNA Polymerase (New England Biolabs, Beverly, Mass.). Amplification is carried out with an initial denaturation at 98° C. for 1 min, followed by 30 cycles of denaturation at 98° C. for 10 sec, annealing at 62° C. for 30 sec, elongation at 72° C. for 20 sec and a final elongation cycle at 72° C. for 5 min, followed by a 4° C. hold. Primer N695A containes an AvrII restriction site for cloning and a RBS upstream of the ATG start codon of the sadB coding region. The N696A primer includes an XbaI site for cloning. The 1.1 kb PCR product is digested with AvrII and XbaI (New England Biolabs, Beverly, Mass.) and gel purified using a Qiaquick Gel Extraction Kit (Qiagen Inc., Valencia, Calif.)). The purified fragment is ligated with pTrc99A::budB-ilvC-ilvD-kivD, that has been cut with the same restriction enzymes, using T4 DNA ligase (New England Biolabs, Beverly, Mass.). The ligation mixture is incubated at 16° C. overnight and then transformed into E. coli Mach 1™ competent cells (Invitrogen) according to the manufacturer's protocol. Transformants are obtained following growth on the LB agar plates with 100 μg/mL ampicillin. Plasmid DNA from the transformants is prepared with QIAprep Spin Miniprep Kit (Qiagen Inc., Valencia, Calif.) according to manufacturer's protocols. The resulting plasmid is called pTrc99A::budB-ilvC-ilvD-kivD-sadB. Electrocompetent E. coli K12 MG1655 Δgnd cells are prepared as described and transformed with pTrc99A::budB-ilvC-ilvD-kivD-sadB. Transformants are streaked onto LB agar plates containing 100 μg/mL ampicillin. The resulting strain is E. coli K12 MG1655 Δgnd carrying plasmid pTrc99A::budB-ilvC-ilvD-kivD-sadB, and is designated E. coli K12 MG1655 Δgnd iso⁺.

### Example 3

### Prophetic

### Expression of gsda from A. niger in E. coli K12 MG1655 Δgnd iso⁺

Expression from of a set of heterologous genes on a second plasmid in addition to genes encoding an isobutanol production pathway in an E. coli or E. coli gene deletion strain can be carried out by standard molecular biology techniques known in the art. As an example it is described how to clone and express in E. coli K12 MG1655 Δgnd pTrc99A::budB-ilvC-ilvD-kivD-sadB the gsdA gene that encodes a glucose-6-phosphate dehydrogenase enzyme (EC 1.1.1.49) from Aspergillus niger. The gene is codon-optimized and synthesized by DNA 2.0 based on the provided amino acid sequence (SEQ ID No118). Restriction sites are added to the sequence during synthesis to allow facile subcloning of the gene into the expression vector. Immediately upstream and adjacent to the translational ATG start codon a HindIII restriction site (AAGCTT) and immediately downstream and adjacent to the TAA translational stop codon Agel restriction sites (ACCGGT) are included. The expression vector is a spectinomycin-resistant plasmid pCL1925 (U.S. Pat. No. 7,074,608) containing the glucose isomerase promoter from Streptomcyes. Vector pCL1925 is digested with HindIII and Agel and the 4.5 kbp vector fragment gel-purified. The gsdA plasmid from DNA 2.0 is digested with the same enzymes to release a 1.5 kbp insert fragment that is gel purified. The vector DNA and insert DNA fragments are ligated with T4 DNA ligase (New England Biolabs, Beverly, Mass.) overnight at 16° C. The ligation is transformed into E. coli K12 MG1655 Δgnd and spread onto LB plates containing 50 μg/mL spectinomycin at 37° C. Transformants are screened by colony PCR as described previously with primers to the vector that flank the insert, pCL1925 vec F (SEQ ID No. 235) and pCL1925 vec R1 (SEQ ID No 236). Plasmids that produce the expected 1.9 kbp product are named pCL1925-gsdA. E. coli K12 MG1655 Δgnd carrying pTrc99A::budB-ilvC-ilvD-kivD-sadB is grown in LB medium containing ampicillin (100 μg/mL) overnight with shaking at 37° C. Overnight cultures are subcultured into the same medium and grown to an OD₆₀₀ of 0.5-0.7 and then harvested by centrifugation to prepare electrocompetent cells. Electrocompetent cells of E. coli K12 MG1655 Δgnd carrying pTrc99A::budB-ilvC-ilvD-kivD-sadB are prepared as described by Ausubel, F. M., et al. (Current Protocols in Molecular Biology, 1987, Wiley-Interscience). Electrocompetent cells are transformed with pCL1925-gsdA. Transformants are spread onto LB agar plates containing 100 μg/mL ampicillin and 50 μg/mL spectinomycin. The resulting strain E. coli K12 MG1655 Δgnd is carrying the isobutanol production plasmid, pTrc99A::budB-ilvC-ilvD-kivD-sadB and the vector pCL1925-gsdA.

### Example 4

### Prophetic

### Production of Isobutanol in E. coli Expressing EDP Genes

Following construction of an E. coli K12 MG1655 strain carrying the isobutanol production plasmid, pTrc99A::budB-ilvC-ilvD-kivD-sadB, in another step genes encoding enzymes that catalyze phosphogluconate dehydratase reaction (EC 4.2.1.12) and 2-dehydro-3-deoxy-phosphogluconate aldolase reaction (EC 4.1.2.14) are cloned and expressed in E. coli K12 MG1655 pTrc99A::budB-ilvC-ilvD-kivD-sadB using methods described above.

The gene that encodes phosphogluconate dehydratase reaction (EC 4.2.1.12) is chosen from E. coli, specifically GenBank No: NP_{—}416365.1 (DNA SEQ ID NO:139, Protein SEQ ID: 140 (str. K12 substr. MG1655), and is designated edp3.

The gene that encodes 2-dehydro-3-deoxy-phosphogluconate aldolase reaction (EC 4.1.2.14) is chosen from E. coli, specifically GenBank No: NP_{—}416364.1 (DNA SEQ ID NO: 208, Protein SEQ ID NO: 209), and is designated edp4.

In another step, endogenous E coli K12 MG1655 genes encoding 6-phosphofructokinase reaction (EC 2.7.1.11), especially genes pfkA (DNA SEQ ID NO: 165, Protein SEQ ID NO: 166)) and pfkB (DNA SEQ ID NO: 163, Protein SEQ ID NO: 164), fructose-bisphosphate aldolase reaction (EC 4.1.2.13), especially genes fbaA (DNA SEQ ID NO: 179, Protein SEQ ID NO: 180) and fbaB (DNA SEQ ID NO: 177, Protein SEQ ID NO: 178), and 6-phosphogluconate reaction (EC 1.1.1.44), especially gnd (DNA SEQ ID NO: 143, Protein SEQ ID NO: 144), are deleted by tools described above.

Strain E. coli K12 MG1655 Δgnd ΔpfkA ΔpfkB ΔfbaA ΔfbaB pTrc99A::budB-ilvC-ilvD-kivD-sadB pCL1925-edp3-edp4 is constructed by methods and tools described above. Strain E. coli K12 MG1655 Δgnd ΔpfkA ΔpfkB ΔfbaA ΔfbaB pTrc99A::budB-ilvC-ilvD-kivD-sadB pCL1925-edp3-edp4 is inoculated into a 250 mL shake flask containing 50 mL of LB-medium, 100 μg/mL ampicillin and 50 μg/mL spectinomycin and shaken at 250 rpm and 37° C. The shake flask is closed with a screw cap to prevent gas exchange with environment. After 24 hours, an aliquot of the broth is analyzed by HPLC (as described above for isobutanol content. Isobutanol is detected.

### Example 5

### Prophetic

### Deletion of 6-phosphogluconate Dehydrogenase Genes in Saccharomyces cerevisiae

The GND1 gene, encoding a first isozyme of 6-phosphogluconate dehydrogenase, is disrupted by insertion of a LEU2 marker cassette by homologous recombination, which completely removes the endogenous GND1 coding sequence. The LEU2 marker in pRS425 (ATCC No. 77106) is PCR-amplified from plasmid DNA using Phusion DNA polymerase (New England Biolabs Inc., Beverly, Mass.; catalog no. F-540S) using primers 4219-T7 and 4219-T8, given as SEQ ID NOs: 237 and 238 which generates a ˜1.8 kb PCR product. The GND1 portion of each primer is derived from the 5′ region upstream of the GND2 promoter and 3′ region downstream of the transcriptional terminator, such that integration of the LEU2 marker results in replacement of the GND1 coding region. The PCR product is transformed into S. cerevisiae BY4741 (ATCC # 201388) using standard genetic techniques (Methods in Yeast Genetics, 2005, Cold Spring Harbor Laboratory Press, Cold Spring Harbor, N.Y., pp. 201-202) and transformants are selected on synthetic complete media lacking leucine and supplemented with 2% glucose at 30° C. Transformants are screened by PCR using primers 4219-T9 and 4219-T10, given as SEQ ID NOs: 239 and 240, to verify integration at the GND1 chromosomal locus with replacement of the GND1 coding region. The identified correct transformants have the genotype: BY4741 gnd1::LEU2.

The GND2 gene, encoding the second isozyme of 6-phosphogluconate dehydrogenase, is disrupted by insertion of a URA3 marker cassette by homologous recombination, which completely removes the endogenous GND2 coding sequence. The URA3 marker in pRS426 (ATCC No. 77107) is PCR-amplified from plasmid DNA using Phusion DNA polymerase (New England Biolabs Inc., Beverly, Mass.; catalog no. F-5405) using primers 4219-T11 and 4219-T12, given SEQ ID NOs 241 and 242, which generates a ˜1.4 kb PCR product. The GND2 portion of each primer is derived from the 5′ region upstream of the GND2 promoter and 3′ region downstream of the transcriptional terminator, such that integration of the URA3 marker results in replacement of the GND2 coding region. The PCR product is transformed into S. cerevisiae BY4741 (ATCC #201388) using standard genetic techniques (Methods in Yeast Genetics, 2005, Cold Spring Harbor Laboratory Press, Cold Spring Harbor, N.Y., pp. 201-202) and transformants are selected on synthetic complete media lacking uracil and supplemented with 2% glucose at 30° C. Transformants are screened by PCR using primers 4219-T13 and 4219-T14, given as SEQ ID NO: 243 and 244, to verify integration at the GND2 chromosomal locus with replacement of the GND2 coding region. The identified correct transformants have the genotype: BY4741 gnd2::URA3. The URA3 marker is disrupted by plating on 5-fluorootic acid (5FOA; Zymo Research, Orange, Calif.) using standard yeast techniques (Methods in Yeast Genetics, 2005, Cold Spring Harbor Laboratory Press, Cold Spring Harbor, N.Y.) producing strains BY4741 Δgnd2.

### Example 6

### Prophetic

### Expression of Isobutanol Production Pathway in S. cerevisiae

The purpose of this prophetic example is to describe how to obtain isobutanol production in a yeast strain in which the 6-phosphogluconate dehydrogenase activity has been disrupted. Construction of vectors pRS423::CUP1p-alsS+FBAp-ILV3 and pHR81::FBAp-ILV5-GPMp-kivD is described in US Patent Publication # US20070092957 A1, Example 17. pRS423::CUP1p-alsS+FBAp-ILV3 has a chimeric gene containing the CUP1 promoter (SEQ ID NO:218), the alsS coding region from Bacillus subtilis (SEQ ID NO:1), and CYC1 terminator (SEQ ID NO:219) as well as a chimeric gene containing the FBA promoter (SEQ ID NO: 220), the coding region of the ILV3 gene of S. cerevisiae (SEQ ID NO:7), and the ADH1 terminator (SEQ ID NO:222). pHR81::FBAp-ILV5+GPMp-kivD is the pHR81 vector (ATCC #87541) with a chimeric gene containing the FBA promoter, the coding region of the ILV5 gene of S. cerevisiae (SEQ ID NO:223), and the CYC1 terminator as well as a chimeric gene containing the GPM promoter (SEQ ID NO:224), the coding region from kivD gene of Lactococcus lactis (DNA SEQ ID NO:225, Protein SEQ ID NO: 226), and the ADH1 terminator. pHR81 has URA3 and leu2-d selection markers.

Plasmid vector pRS423::CUP1p-alsS+FBAp-ILV3, pHR81::FBAp-ILV5+GPMp-kivD is transformed into strain BY4741 using standard genetic techniques (Methods in Yeast Genetics, 2005, Cold Spring Harbor Laboratory Press, Cold Spring Harbor, N.Y.) and maintained on synthetic complete media lacking histidine and uracil, and supplemented with 2% glucose. Aerobic cultures are grown in 250 mL flasks containing 50 mL synthetic complete media lacking histidine and uracil, and supplemented with 2% glucose in an Innova4000 incubator (New Brunswick Scientific, Edison, N.J.) at 30° C. and 225 rpm. The strain is referred to as S. cerevisiae iso⁺.

### Example 7

### Prophetic

### Expression of GSDA from Aspeqillus niger in Saccharomyces cerevisiae iso⁺

The purpose of this prophetic example is to describe how to obtain an isobutanol producing yeast strain that is disrupted for 6-phosphogluconate dehydrogenase activity, and expresses glucose-6-phosphate dehydrogenase GSDA of Aspergillus niger in the cytosol of S. cerevisiae. Plasmid pRS411 (Brachmann, C B, et al. 1998, Yeast 14:115-132; available from American Type Culture Collection (“ATCC”), Manassas, Va., #87474) will be used for expression of the enzyme.

The codon-optimized nucleotide sequence encoding the glucose-6-phosphate dehydrogenase from A. niger gsdA protein (SEQ ID NO: 118) is synthesized by DNA 2.0 (Menlo Park, Calif.), based on the provided amino acid sequence (SEQ ID NO: 117). A cloned DNA fragment containing the optimized coding region called gsdA_opt is received from DNA 2.0.

Next a chimeric gene containing the GPM promoter-gsdA_opt coding region-ADH1terminator is constructed as follows. The gsdA_opt coding region is PCR amplified from plasmid template (supplied from DNA 2.0) using primers 4219-T3 and 4219-T4 (SEQ ID NOs: 245 and 246) that contain additional 5′ sequences that overlap with the yeast GPM1 promoter and ADH1 terminator. The S. cerevisiae GPM1 promoter is PCR amplified from BY4743 genomic DNA (ATCC 201390) using primers 4219-T1 and 4219-T2 (SEQ ID NOs: 247 and 248) that contain additional 5′ sequences that overlap with the pRS411 vector and the gsdA_opt coding region. The S. cerevisiae ADH1 terminator is PCR amplified from BY4743 genomic DNA using primers 4219-T5 and 4219-T6 (SEQ ID NOs: 249 and 225) that contain additional 5′ sequences that overlap with the gsdA_opt coding region and pRS411 vector sequence. The PCR products are then assembled using “gap repair” methodology in S. cerevisiae (Ma et al., Gene, 58: 201-216, 1987).

The yeast-E. coli shuttle vector pRS411 is linearized by digestion with KpnI SacI restriction enzymes and purified by gel electrophoresis. Approximately 1.0 μg of the purified pRS411 backbone is co-transformed with 1.0 μg of gsdA_opt PCR product and 1.0 μg of GPM1 promoter PCR product, and 1 μg of ADH1 terminator PCR product into S. cerevisiae BY4641. Transformants are selected on the synthetic complete medium lacking methionine and supplemented with 2% glucose at 30° C. The proper recombination event, generating pRS411::GPM-gsdA-ADH1t, is confirmed by DNA sequencing (SEQ ID NO: 226).

### Example 8

### Prophetic

### Production of Isobutanol in S. cerevisiae Expressing EDP

Following construction of a S. cerevisiae iso⁺ strain carrying the isobutanol production plasmid, pRS423::CUP1p-alsS+FBAp-ILV3 and pHR81::FBAp-ILV5+GPMp-kivD as described above, in another step, genes encoding enzymes that catalyze glucose-6-phosphate dehydrogenase reaction (EC 1.1.1.49), phosphogluconate dehydratase reaction (EC 4.2.1.12), and 2-dehydro-3-deoxy-phosphogluconate aldolase reaction (EC 4.1.2.14) are cloned and expressed in S. cerevisiae iso⁺ by methods and tools described above.

The gene that encodes glucose-6-phosphate dehydrogenase reaction (EC 1.1.1.49) is from Aspergillus nidulans, specifically GenBank No: XP_{—}660585.1 (DNA SEQ ID NO: 119, Protein SEQ ID NO:120), and is referred to as edp1.

The gene that encodes phosphogluconate dehydratase reaction (EC 4.2.1.12) is chosen from Pseudomonas putida, specifically GenBank No: NP_{—}743171.1 (DNA SEQ ID NO: 137, Protein SEQ ID NO:138), and is referred to as edp3.

The gene that encodes 2-dehydro-3-deoxy-phosphogluconate aldolase reaction (EC 4.1.2.14) is chosen from Pseudomonas fluorescens, specifically GenBank No: YP_{—}261692.1 (DNA SEQ ID NO: 204, Protein SEQ ID NO: 205), and is referred to as edp4.

In another step, endogenous genes of S. cerevisiae encoding 6-phosphofructokinase reaction (EC 2.7.1.11), especially genes PFK1 (DNA SEQ ID NO: 171, Protein SEQ ID NO:172) and PFK2 (DNA SEQ ID NO: 173, Protein SEQ ID NO:174), fructose-bisphosphate aldolase reaction (EC 4.1.2.13), especially gene FBA1 (DNA SEQ ID NO: 185, Protein SEQ ID NO:186-phosphogluconate dehydrogenase reaction (EC 1.1.1.44), especially genes GND1 (DNA SEQ ID NO: 149, Protein SEQ ID NO:150) and GND2 (DNA SEQ ID NO: 147, Protein SEQ ID NO:148), are deleted by methods well known in the art.

Strain S. cerevisiae ΔGND1 ΔGND2 ΔPFK1 ΔPFK2 ΔFBA1 iso+ pRS411::GPM-edp3-edp4-edp2 is constructed by methods and tools described above. Strains were maintained on standard S. cerevisiae synthetic complete medium (Methods in Yeast Genetics, 2005, Cold Spring Harbor Laboratory Press, Cold Spring Harbor, N.Y., pp. 201-202) containing 2% glucose but lacking methionine, uracil and histidine to ensure maintenance of plasmids. The strain is inoculated into an aerobic 250 mL flasks containing 50 ml synthetic complete media lacking histidine and methionine, and supplemented with 2% glucose in an Innova 4000 incubator (New Brunswick Scientific, Edison, N.J.) at 30° C. and 225 rpm. Low oxygen cultures are prepared by adding 45 mL of medium to 60 mL serum vials that are sealed with crimped caps after inoculation and kept at 30° C. Approximately 24 and 48 hours after induction with 0.03 mM CuSO₄ (final concentration), an aliquot of the broth is analyzed by HPLC as described above for isobutanol content. Isobutanol is detected.

### Example 9

### Prophetic

### Deletion of 6-phosphogluconate Dehydrogenase in Lactobacillus plantarum PN0512

The purpose of this section is to describe the deletion of the gnd1 gene (SEQ NO: 151) in Lactobacillus plantarum PN0512 to create strain Lactobacillus plantarum PN0512 Δgnd1.

The Δgnd1 deletion is constructed by a two-step homologous recombination procedure, described above, utilizing a shuttle vector, pFP996. The homologous DNA arms are 1200 bp each and are designed such that the deletion would encompass 497 nucleotides of the gnd1 gene, leaving the first and last 200 nucleotides of the gene intact. The gnd1 left homologous arm is amplified from L. plantarum PN0512 genomic DNA with primers gnd-left-arm-up (SEQ ID NO: 183), containing a BglII site, and gnd-left-arm-down (SEQ ID NO: 158), containing a KpnI site. The gnd1 right homologous arm is amplified from L. plantarum PN0512 genomic DNA with primers gnd-right-arm-up [SEQ ID NO: 182], containing a KpnI site, and gnd-right-arm-down [SEQ ID NO: 181], containing a BsrGI site. The gnd1 left homologous arm is digested with BglII and KpnI and the gnd1 right homologous arm is digested with KpnI and BsrGI. The two homologous arms are ligated with T4 DNA Ligase into the corresponding restriction sites of pFP996, after digestion with the appropriate restriction enzymes, to generate the vector pFP996-gnd1-arms.

The following procedure is used to generate the deletion: Lactobacillus plantarum PN0512 is transformed with the pFP996-gnd1-arms construct by the following procedure. 5 mL of Lactobacilli MRS medium (Accumedia, Neogen Corporation, Lansing, Mich.) is inoculated with PN0512 and grown overnight at 30° C. 100 mL MRS medium is inoculated with overnight culture to an OD₆₀₀ 0.1 and grown to an OD₆₀₀ 0.7 at 30° C. Cells are harvested at 3700×g for 8 min at 4° C., washed with 100 mL cold 1.0 mM MgCl₂ (Sigma-Aldrich, St. Louis, Mo.), centrifuged at 3700×g for 8 min at 4° C., washed with 100 mL cold 30% PEG-1000 (Sigma-Aldrich, St. Louis, Mo.), recentrifuged at 3700×g for 20 min at 4° C., then resuspended in 1.0 mL cold 30% PEG-1000. 60 μL cells are mixed with ˜100 ng plasmid DNA in a cold 1 mm gap electroporation cuvette and electroporated in a BioRad Gene Pulser (Hercules, Calif.) at 1.7 kV, 25 μF, and 400Ω. Cells are resuspended in 1.0 mL MRS medium containing 500 mM sucrose (Sigma-Aldrich, St. Louis, Mo.) and 100 mM MgCl₂, incubated at 30° C. for 2 hours, and then plated on MRS medium plates containing 2 μg/mL of erythromycin (Sigma-Aldrich, St. Louis, Mo.).

The presence of the plasmid in transformants is confirmed by colony PCR using plasmid specific primers oBP42 [SEQ ID 170] and oBP57 [SEQ ID 169].

Transformants are grown at 30° C. in Lactobacilli MRS medium with erythromycin (3 μg/mL) for approximately 10 generations. Transformants are then grown at 37° C. for approximately 50 generations by serial inoculations in Lactobacilli MRS medium. Cultures are plated on Lactobacilli MRS medium with erythromycin (1 μg/mL). Isolates are screened by colony PCR for a single crossover with chromosomal specific primer gnd1-check-up [SEQ ID 168] and plasmid specific primer oBP42 [SEQ ID 170]. Single crossover integrants are grown at 37° C. for approximately 40 generations by serial inoculations in Lactobacilli MRS medium.

Cultures are streaked on the MRS-containing plates and isolates are patched to MRS plates, grown at 37° C., and then patched onto MRS medium with erythromycin (1 μg/mL).

Erythromycin sensitive isolates are screened by colony PCR for the presence of a wild-type or deletion second crossover using chromosomal specific primers gnd1-check-up [SEQ ID 168] and gnd1-check-down [SEQ ID 167]. A wild-type sequence yields a 3097 by product and a deletion sequence yields a 2600 by product. The deletion is confirmed by sequencing the PCR product. The absence of plasmid is tested by colony PCR using plasmid specific primers oBP42 [SEQ ID 170] and oBP57 [SEQ ID 169].

### Example 10

### Prophetic

### Expression of Isobutanol Production Pathway in Lactobacillus plantarum PN0512

The purpose of this section is to describe the construction of an isobutanol production plasmid expressing a heterologous dihydroxyacid dehydratase, ketol-acid reductoisomerase, α-ketoisovalerate decarboxylase, alcohol dehydrogenase, and acetolactate synthase. The genes are expressed on a shuttle vector pDM1 (SEQ 157). Plasmid pDM1 contains a minimal pLF1 replicon (˜0.7 Kbp) and pemK-peml toxin-antitoxin(TA) from Lactobacillus plantarum ATCC14917 plasmid pLF1, a P15A replicon from pACYC184, chloramphenicol resistance marker for selection in both E. coli and L. plantarum, and P30 synthetic promoter [Rud et al, Microbiology, 152:1011-1019, 2006].

Genomic DNA for PCR is prepared with MasterPure DNA Purification Kit (Epicentre, Madison, Wis.) following the recommended protocol. Codon-optimized nucleotide sequences, supplied on plasmids, are synthesized by DNA 2.0 (Menlo Park, Calif.), based on provided amino acid sequences.

The ilvD gene from Lactococcus lactis subsp. lactis (SEQ ID 109) encoding dihydroxyacid dehydratase (SEQ ID 110) is amplified from genomic DNA with primer ilvD-up (SEQ ID 129), containing a PstI restriction site and ribosome binding site, and primer ilvD-down (SEQ ID 130), containing a DrdI restriction site. The resulting PCR product and pDM1 are ligated after digestion with PstI and DrdI to yield vector pDM1-ilvD with the ilvD gene immediately downstream of the P30 promoter. The IdhL1 promoter region of Lactobacillus plantarum PN0512 (SEQ ID 250) is amplified from genomic DNA with primer PldhL1-up (SEQ ID 145), containing a DrdI restriction site, and primer PldhL1-down (SEQ ID 146), containing BamHI, SacI, PacI, NotI, SalI, and DrdI restriction sites. The resulting PCR product and vector pDM1-ilvD are ligated after digestion with DrdI. Clones are screened by PCR for inserts that are in the same orientation as the ilvD gene using primers ilvD-up (SEQ ID 129) and PldhL1-down (SEQ ID 130). A clone that has the correctly oriented insert is designated pDM1-ilvD-PldhL1. The ilvC gene from Bacillus subtilis, codon optimized for expression in Lactobacillus plantarum (SEQ ID 251), encoding ketol-acid reductoisomerase (SEQ ID 14) is amplified from plasmid DNA (DNA 2.0, see above) with primers ilvC-up (SEQ ID 192), containing a BamHI restriction site and ribosome binding site, and ilvC-down (SEQ ID 193), containing a SacI restriction site. The resulting PCR product and vector pDM1-ilvD-PldhL1 are ligated after digestion with BamHI and SacI to yield vector pDM1-ilvD-PldhL1-ilvC. The kivD gene from Lactococcus lactis subsp. lactis (SEQ ID 189) encoding α-ketoisovalerate decarboxylase (SEQ ID 26) is amplified from genomic DNA with primers kivD-up (SEQ ID 252), containing a SacI restriction site and ribosome binding site, and kivD-down (SEQ ID:253) containing a PacI restriction site. The resulting PCR product and pDM1-ilvD-PldhL1-ilvC are ligated after digestion with SacI and PacI to yield vector pDM1-ilvD-PldhL1-ilvC-kivD. The sadB gene from Achromobacter xylosoxidans (SEQ ID 103) encoding a secondary alcohol dehydrogenase (SEQ ID 104) is amplified from genomic DNA with primers sadB-up (SEQ ID 210), containing a PacI restriction site and ribosome binding site, and sadB-down (SEQ ID 211), containing a NotI restriction site. The resulting PCR product and pDM1-ilvD-PldhL1-ilvC-kivD are ligated after digestion with PacI and NotI to yield vector pDM1-ilvD-PldhL1-ilvC-kivD-sadB. The alsS gene from Bacillus subtilis, codon optimized for expression in Lactobacillus plantarum (SEQ ID 254), encoding acetolactate synthase (SEQ ID 2) is amplified from plasmid DNA (DNA 2.0, see above) with primers alsS-up (SEQ ID 255), containing a NotI restriction site and ribosome binding site, and alsS-down (SEQ ID 256), containing a SalI restriction site. The resulting PCR product and pDM1-ilvD-PldhL1-ilvC-kivD-sadB are ligated after digestion with NotI and SalI to yield the isobutanol vector pDM1-ilvD-PldhL1-ilvC-kivD-sadB-alsS. Lactobacillus plantarum strain PN0512 is transformed with vector pDM1-ilvD-PldhL1-ilvC-kivD-sadB-alsS as described above. Transformants are selected on MRS medium containing chloramphenicol (10 μg/ml) and result in strain PN0512 pDM1-ilvD-PldhL1-ilvC-kivD-sadB-alsS. The strain contains all five genes of the isobutanol pathway on the plasmid.

### Example 11

### Prophetic

### Expression of gsdA from Aspergillus niger in Lactobacillus plantarum PN0512 iso⁺

The purpose of this prophetic example is to describe how to obtain an isobutanol producing Lactobacillus plantarum strain that expresses glucose-6-phosphate dehydrogenase GSDA of Aspergillus niger.

Vector pFP996PIdhL1 (SEQ ID NO: 142) is a shuttle vector with two origins of replication and two selectable markers which allow for replication and selection in both E. coli and L. plantarum. The vector contains the promoter region from the Lactobacillus plantarum PN0512 IdhL1 gene for expression of genes in L. plantarum. The A. niger gsdA gene encoding glucose-6-phosphate dehydrogenase is amplified with primers FP996-gsdA-up [SEQ ID 141], containing an XmaI site and a ribosome binding site, and FP996-gsdA-down [SEQ ID 184], containing a KpnI site. The template for the PCR reaction is plasmid DNA containing the A. niger gsdA coding sequence [SEQ ID 117] which is synthesized by DNA 2.0 (Manlo Park, Calif.). The resulting PCR fragment and pFP996PIdhL1 are ligated after digestion with XmaI and KpnI to create vector pFP996PIdhL1-gsdA(An).

L. plantarum strain PN0512 pDM1-ilvD-PldhL1-ilvC-kivD-sadB-alsS is transformed with vector pFP996PIdhL1-gsdA(An) as described above. Transformants are selected on MRS medium containing erythromycin (3 μg/mL) and chloramphenicol (10 μg/mL) and result in strain L. plantarum PN0512 pDM1-ilvD-PldhL1-ilvC-kivD-sadB-alsS pFP996PldhL1-gsdA(An).

### Example 12

### Prophetic

### Production of Isobutanol in L. plantarum Expressing EDP

Following construction of an L. plantarum PN0512 strain carrying the isobutanol production plasmid, pDM1-ilvD-PldhL1-ilvC-kivD-sadB-alsS, in another step genes encoding enzymes that catalyze glucose-6-phosphate dehydrogenase reaction (EC 1.1.1.49), phosphogluconate dehydratase reaction (EC 4.2.1.12), and 2-dehydro-3-deoxy-phosphogluconate aldolase reaction (EC 4.1.2.14) are cloned and expressed in L. plantarum PN0512 pDM1-ilvD-PldhL1-ilvC-kivD-sadB-alsS by tools described above.

The gene encoding glucose-6-phosphate dehydrogenase reaction (EC 1.1.1.49) is from Aspergillus niger, specifically GenBank No: CAA61194.1 (DNA SEQ ID NO:117, Protein SEQ ID NO:118) and is referred to as edp1.

The gene that encodes phosphogluconate dehydratase reaction (EC 4.2.1.12) is chosen from Zymomonas mobilis, specifically GenBank No: YP_{—}162103.1 (DNA SEQ ID NO:135, Protein SEQ ID: 136) and are referred to as edp3.

The gene that encodes 2-dehydro-3-deoxy-phosphogluconate aldolase reaction (EC 4.1.2.14) is from Pseudomonas putida, specifically GenBank No: NP_{—}743185.1 (DNA SEQ ID NO: 202, Protein SEQ ID NO: 203) and is referred to as edp4.

In another step, genes that encode the endogenous, chromosomal glucose-6-phosphate dehydrogenase reaction (EC 1.1.1.49), especially gene zwf (DNA SEQ ID NO: 131, Protein SEQ ID NO: 132), 6-phosphofructokinase reaction (EC 2.7.1.11), especially gene pfkA (DNA SEQ ID NO: 175, Protein SEQ ID NO: 176), fructose-bisphosphate aldolase reaction (EC 4.1.2.13), especially genes fba (DNA SEQ ID NO: 187, Protein SEQ ID NO: 188), 6-phospho-gluconate dehydrogenase reaction (EC 1.1.1.44), especially genes gnd1 (DNA SEQ ID NO: 151, Protein SEQ ID NO: 152) and gnd2 (DNA SEQ ID NO: 153, Protein SEQ ID NO: 154), are deleted by tools described above.

Strain L. plantarum PN0512 Δgnd1 Δgnd2 Δzwf ΔpfkA Δfba pDM1-ilvD-PldhL1-ilvC-kivD-sadB-alsS pFP996PldhL1-edp1-edp3-edp4 is constructed by methods and tools described above. Strain L. plantarum PN0512 Δgnd1 Δgnd2 Δzwf ΔpfkA Δfba pDM1-ilvD-PldhL1-ilvC-kivD-sadB-alsS pFP996PIdhL1-edp1-edp3-edp4 is grown overnight in Lactobacilli MRS medium with 10 μg/ml chloramphenicol and 3 μg/ml erythromycin at 30° C. as described above and the culture supernatant is analyzed by HPLC for isobutanol content. Isobutanol is detected.

### Example 13

### Construction of an E. coli Strain Having Deletions of pfIB, frdB, IdhA, adhE, qnd, pfkA, pfkB, fbaA and fbaB Genes

This Example describes engineering of an E. coli strain in which nine genes were inactivated. The Keio collection of E. coli strains (Baba et al., Mol. Syst. Biol., 2:1-11, 2006) was used for production of eight of the knockouts. The Keio collection (available from NBRP at the National Institute of Genetics, Japan) is a library of single gene knockouts created in strain E. coli BW25113 by the method of Datsenko and Wanner (Datsenko, K. A. & Wanner, B. L., Proc Natl Acad. Sci., USA, 97: 6640-6645, 2000). In the collection, each deleted gene was replaced with a FRT-flanked kanamycin marker that was removable by Flp recombinase. The E. coli strain carrying multiple knockouts was constructed by moving the knockout-kanamycin marker from the Keio donor strain by bacteriophage P1 transduction to a recipient strain. After each P1 transduction to produce a knockout, the kanamycin marker was removed by Flp recombinase. This markerless strain acted as the new recipient strain for the next P1 transduction. One of the described knockouts was constructed directly in the strain using the method of Datsenko and Wanner (supra) rather than by P1 transduction.

The 4KO E. coli strain was constructed in the Keio strain JW0886 by P1ᵥᵢᵣ transductions with P1 phage lysates prepared from three Keio strains. The Keio strains used are listed below:
JW0886: the kan marker is inserted in the pflB
JW4114: the kan marker is inserted in the frdB
JW1375: the kan marker is inserted in the IdhA
JW1228: the kan marker is inserted in the adhE

To construct the final strain the Keio strains listed below were also utilized as a source of the inactivated genes:
JW2011: the kan marker is inserted in the gnd
JW3887: the kan marker is inserted in the pfkA
JW5280: the kan marker is inserted in the pfkB
JW5344: the kan marker is inserted in the fbaB

[Sequences corresponding to the inactivated genes are: pflB (SEQ ID NO: 260), frdB (SEQ ID NO: 264), IdhA (SEQ ID NO: 272), adhE (SEQ ID NO: 270), gnd (SEQ ID NO:143), pfkA (Seq ID NO: 165), pfkB (SEQ ID NO: 163), and fbaB (SEQ ID NO: 177).] Additionally the fbaA gene (SEQ ID NO: 179) was inactivated in the final strain. The fbaA gene deletion is not in the Keio collection. The fbaA gene was inactivated directly in the final strain using the Datsenko and Wanner method (supra), except a loxP-flanked kanamycin marker was used instead of a FRT flanked kanamycin marker to replace the native gene.

Removal of the FRT-flanked kanamycin marker from the chromosome was performed by transforming the kanamycin-resistant strain with pCP20 an ampicillin-resistant plasmid (Cherepanov, and Wackernagel, supra)). Transformants were spread onto LB plates containing 100 μg/mL ampicillin. Plasmid pCP20 carries the yeast FLP recombinase under the control of the γ_{P}_{_{R}} promoter and expression from this promoter is controlled by the c1857 temperature-sensitive repressor residing on the plasmid. The origin of replication of pCP20 is also temperature-sensitive.

Removal of the loxP-flanked kanamycin marker from the chromosome was performed by transforming the kanamycin-resistant strain with pJW168 an ampicillin-resistant plasmid (Wild et al., Gene. 223:55-66, 1998) harboring the bacteriophage P1 Cre recombinase. Cre recombinase (Hoess, R. H. & Abremski, K., supra) meditates excision of the kanamycin resistance gene via recombination at the loxP sites. The origin of replication of pJW168 is the temperature-sensitive pSC101. Transformants were spread onto LB plates containing 100 μg/mL ampicillin.

Strain JW0886 (ΔpflB::kan) was transformed with plasmid pCP20 and spread on the LB plates containing 100 μg/mL ampicillin at 30° C. Ampicillin resistant transformants were then selected, streaked on the LB plates and grown at 42° C. Isolated colonies were patched onto the ampicillin and kanamycin selective medium plates and LB plates. Kanamycin-sensitive and ampicillin-sensitive colonies were screened by colony PCR with primers pflB CkUp (SEQ ID NO: 297) and pflB CkDn (SEQ ID NO: 298). A 10 μL aliquot of the PCR reaction mix was analyzed by gel electrophoresis. The expected approximate 0.4 kb PCR product was observed confirming removal of the marker and creating the “JW0886 markerless” strain. This strain has a deletion of the pflB gene.

The “JW0886 markerless” strain was transduced with a P1ᵥᵢᵣ lysate from JW4114 (frdB::kan) and streaked onto the LB plates containing 25 μg/mL kanamycin. The kanamycin-resistant transductants were screened by colony PCR with primers frdB CkUp (SEQ ID NO: 299) and frdB CkDn (SEQ ID NO: 300). Colonies that produced the expected approximate 1.6 kb PCR product were made electrocompetent and transformed with pCP20 for marker removal as described above. Transformants were first spread onto the LB plates containing 100 μg/mL ampicillin at 30° C. and ampicillin resistant transformants were then selected and streaked on LB plates and grown at 42° C. Isolated colonies were patched onto ampicillin and the kanamycin selective medium plates and LB plates. Kanamycin-sensitive, ampicillin-sensitive colonies were screened by PCR with primers frdB CkUp (SEQ ID NO: 299) and frdB CkDn (SEQ ID NO: 300). The expected approximate 0.4 kb PCR product was observed confirming marker removal and creating the double knockout strain, “ΔpflB frdB”.

The double knockout strain was transduced with a P1ᵥᵢᵣ lysate from JW1375 (ΔldhA::kan) and spread onto the LB plates containing 25 μg/mL kanamycin. The kanamycin-resistant transductants were screened by colony PCR with primers IdhA CkUp (SEQ ID NO: 301) and IdhA CkDn (SEQ ID NO: 302). Clones producing the expected 1.5 kb PCR product were made electrocompetent and transformed with pCP20 for marker removal as described above. Transformants were spread onto LB plates containing 100 μg/mL ampicillin at 30° C. and ampicillin resistant transformants were streaked on LB plates and grown at 42° C. Isolated colonies were patched onto ampicillin and kanamycin selective medium plates and LB plates. Kanamycin-sensitive, ampicillin-sensitive colonies were screened by PCR with primers IdhA CkUp (SEQ ID NO: 301) and IdhA CkDn (SEQ ID NO: 302) for a 0.3 kb product. Clones that produced the expected approximate 0.3 kb PCR product confirmed marker removal and created the triple knockout strain designated “3KO” (ΔpflB frdB IdhA).

Strain “3 KO” was transduced with a P1ᵥᵢᵣ lysate from JW1228 (ΔadhE::kan) and spread onto the LB plates containing 25 μg/mL kanamycin. The kanamycin-resistant transductants were screened by colony PCR with primers adhE CkUp (SEQ ID NO: 303) and adhE CkDn (SEQ ID NO: 304). Clones that produced the expected 1.6 kb PCR product were named 3KO adhE::kan. Strain 3KO adhE::kan was made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers adhE CkUp (SEQ ID NO: 303) and adhE CkDn (SEQ ID NO: 304). Clones that produced the expected approximate 0.4 kb PCR product were named “4KO” (ΔpflB frdB IdhA adhE).

Strain “4 KO” was transduced with a P1ᵥᵢᵣ lysate from JW2011 (Δgnd::kan) and spread onto the LB plates containing 25 μg/mL kanamycin. The kanamycin-resistant transductants were screened by colony PCR with primers gnd CkF (SEQ ID NO: 305) and gnd CkR (SEQ ID NO: 306). Clones that produced the expected 1.6 kb PCR product were named 4KO gnd::kan and were made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers gnd CkF (SEQ ID NO: 305) and gnd CkR (SEQ ID NO: 306). Clones that produced the expected approximate 0.4 kb PCR product were named “4KO gnd” (ΔpflB frdB IdhA adhE gnd).

Strain “4 KO gnd” was transduced with a P1ᵥᵢᵣ lysate from JW3887 (ΔpfkA::kan) and spread onto the LB plates containing 25 μg/mL kanamycin. The kanamycin-resistant transductants were screened by colony PCR with primers pfkA CkF (SEQ ID NO: 307) and pfkA CkR2 (SEQ ID NO: 308). Clones that produced the expected 1.6 kb PCR product were named 5KO pfkA::kan (ΔpflB frdB IdhA adhE gnd pfkA::kan) and were made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers pfkA CkF (SEQ ID NO: 307) and pfkA CkR2 (SEQ ID NO: 308). Clones that produced the expected approximate 0.3 kb PCR product were named “5 KO pfkA” (ΔpflB frdB IdhA adhE gnd pfkA).

Strain “5KO pfkA” was transduced with a P1ᵥᵢᵣ lysate from JW5280 (ΔpfkB::kan) and spread onto the LB plates containing 25 μg/mL kanamycin. The kanamycin-resistant transductants were screened by colony PCR with primers pfkB CkF2 (SEQ ID NO:309) and pfkB CkR2 (SEQ ID NO: 310). Clones that produced the expected 1.7 kb PCR product were named 6KO pfkB::kan (ΔpflB frdB IdhA adhE gnd pfkA pfkB::kan). and made electrocompetent and transformed with pCP20 for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR with the primers pfkB CkF2 (SEQ ID NO: 309) and pfkB CkR2 (SEQ ID NO: 310). Clones that produced the expected approximate 0.5 kb PCR product were named “6KO pfkB::” (ΔpflB frdB IdhA adhE gnd pfkA pfkB).

Gene deletions in E. coli can be carried out by standard molecular biology techniques appreciated by one skilled in the art. To create an fbaA deletion in E. coli “4KO gpp”, the gene is deleted by replacing it with a kanamycin resistance marker using the Lambda Red-mediated homologous recombination system as described by Datsenko and Wanner (supra). PCR amplification with pLoxKan2 (Palmeros et al., Gene 247:255-264, 2000) as template and primers fbaA H1 P1 lox (SEQ ID NO: 311) and fbaA H2 P4 lox (SEQ ID NO: 312) produces a 1.4 kb product. Primer H1 consists of the first 50 by of the CDS of fbaA followed by 22 nucleotides homologous to a binding site upstream of a loxP site in pLoxKan2. The H2 primer consists of the last 43 base pairs of the fbaA CDS and 7 by downstream of the CDS followed by 20 bps homologous to binding site downstream of a loxP site in pLoxKan2. PCR amplification uses the HotStarTaq Master Mix (Qiagen, Valencia, Calif.; catalog no. 71805-3) according to the manufacturer's protocol. Amplification is carried out in a DNA Thermocycler GeneAmp 9700 (PE Applied Biosystems, Foster City, Calif.). PCR conditions were: 15 min at 95° C.; 30 cycles of 95° C. for 30 sec, annealing temperature of 63° C. for 30 sec, an extension time of approximately 1 min/kb of DNA at 72° C.; then 10 min at 72° C. followed by a hold at 4° C. After amplification the PCR reaction is loaded onto a 1% agarose gel in TBE buffer and electrophoresed at 50 volts for approximately 30 minutes. The PCR product is gel-purified from a 1% agarose gel with a Zymoclean Gel Extraction Kit (Zymo Research Corp. Orange, Calif.).

E. coli “6KO pfkB” is made electrocompetent as described by Ausubel, F. M., et al., (Current Protocols in Molecular Biology, 1987, Wiley-Interscience,). and transformed with pKD46, the temperature sensitive Red recombinase plasmid (Datsenko and Wanner, supra). For electroporation a Bio-Rad Gene Pulser II was used according to the manufacturer's instructions (Bio-Rad Laboratories Inc, Hercules, Calif.). After electroporation cells are outgrown in SOC medium (2% Bacto Tryptone (Difco), 0.5% yeast extract (Difco), 10 mM NaCl, 2.5 mM KCL, 10 mM MgCl₂, 10 mM MgSO₄, 20 mM glucose) for 2 hours at 30° C. with shaking. Transformants are spread on LB plates containing 50 μg/ml ampicillin and incubated overnight at 30° C. Transformants are streaked on LB plates containing 50 μg/ml ampicillin and incubated overnight at 30° C. An isolated colony of E. coli “6KO pfkB” carrying pKD46 was grown in 3 ml LB medium with 50 μg/mL ampicillin overnight at 30 C with shaking. One-half milliliter of the overnight culture was diluted into 50 ml LB medium with 50 μg/mL ampicillin and grown at 30 C with shaking. At an OD600 of approximately 0.2. L-arabinose was added to a final concentration of 20 mM and incubation with shaking continued at 30° C. Cells were harvested by centrifugation at an OD600 of 0.5-0.7. Electrocompetent cells of E. coli “6KO pfkB”/pKD46 are then prepared as described above and electrotransformed with up to 1 μg of the 1.4 kb PCR product of the kanamycin marker flanked by loxP sites and homology to fbaA. For electroporation a Bio-Rad Gene Pulser II was used according to the manufacturer's instructions (Bio-Rad Laboratories Inc, Hercules, Calif.). After electroporation cells are outgrown in SOC medium (2% Bacto Tryptone (Difco), 0.5% yeast extract (Difco), 10 mM NaCl, 2.5 mM KCL, 10 mM MgCl₂, 10 mM MgSO₄, 20 mM glucose) for 2 hours at 30° C. with shaking. Transformants are spread onto LB plates containing 25 μg/mL kanamycin and incubated overnight at 42° C. to cure the temperature sensitive Red recombinase plasmid.

Transformants are patched to grids onto LB plates containing kanamycin (25 μg/mL), and LB ampicillin (100 μg/mL) to test for loss of the ampicillin resistant recombinase plasmid, pKD46. Ampillicin-sensitive kanamycin resistant transformants are further analyzed by colony PCR using primers fbaA Ck UP (SEQ ID NO: 313) and fbaA Ck Dn (SEQ ID NO: 314), for the expected 1.5 kb PCR fragment. Clones producing the expected size PCR product were designated E. coli K12 7KO fbaA::kan ((ΔpflB frdB IdhA adhE gnd pfkA pfkB fba::kan).

E. coli K12 7KO fbaA::kan were made electrocompetent and transformed with pJW168 (Wild, et al., supra) for marker removal. Transformants were spread onto the LB plates containing 100 μg/mL ampicillin at 30° C. Ampicillin resistant transformants were streaked on the LB plates and grown at 42° C. Isolated colonies were patched onto ampicillin and kanamycin selective plates and LB plates. Kanamycin-sensitive, ampicillin-sensitive colonies were screened by PCR amplification with the primers fbaA Ck UP (SEQ ID NO: 313) and fbaA Ck Dn (SEQ ID NO: 314) A 10 μL aliquot of the PCR reaction mix was analyzed by gel electrophoresis. Clones that produced the expected approximate 0.3 kb PCR product were named “7KO fbaA” (ΔpflB frdB IdhA adhE gnd pfkA pfkB fbaA).

Strain “7KO fbaA” was transduced with a P1ᵥᵢᵣ lysate from JW5344 (ΔfbaB::kan) and spread onto the LB plates containing 25 μg/mL kanamycin. The kanamycin-resistant transductants were screened by colony PCR with primers fbaB CkF2 (SEQ ID NO: 315) and fbaB CkR2 (SEQ ID NO: 316). Clones that produced the expected 1.6 kb PCR product were named “8KO fbaB::kan (ΔpflB frdB IdhA adhE gnd pfkA pfkB fbaA fbaB::kan).

### Example 14

### Construction of an Isobutanol Biosynthetic Pathway

A DNA fragment encoding sad B, a butanol dehydrogenase, (DNA SEQ ID NO:103; protein SEQ ID NO: 104) from Achromobacter xylosoxidans was amplified from A. xylosoxidans genomic DNA using standard conditions. The DNA was prepared using a Gentra Puregene kit (Gentra Systems, Inc., Minneapolis, Minn.; catalog number D-5500A) following the recommended protocol for gram negative organisms. PCR amplification was done using forward and reverse primers N473 and N469 (SEQ ID NOs: 231 and 232), respectively with Phusion High Fidelity DNA Polymerase (New England Biolabs, Beverly, Mass.). The PCR product was TOPO-Blunt cloned into pCR4 BLUNT (Invitrogen) to produce pCR4Blunt::sadB, which was transformed into E. coli Mach-1 cells. Plasmid was subsequently isolated from four clones, and the sequence verified.

The sadB coding region was then cloned into the vector pTrc99a (Amann et al., Gene 69: 301-315, 1988). The pCR4Blunt::sadB was digested with EcoRI, releasing the sadB fragment, which was ligated with EcoRI-digested pTrc99a to generate pTrc99a::sadB. This plasmid was transformed into E. coli Mach 1 cells and the resulting transformant was named Mach1/pTrc99a::sadB. The activity of the enzyme expressed from the sadB gene in these cells was determined to be 3.5 mmol/min/mg protein in cell-free extracts when analyzed using isobutyraldehyde as the standard.

The sadB gene was then subcloned into pTrc99A::budB-ilvC-ilvD-kivD as described below. The pTrc99A::budB-ilvC-ilvD-kivD is the pTrc-99a expression vector carrying an operon for isobutanol expression (described in Examples 9-14 the of U.S. Published Patent Application No. 20070092957, which are incorporated herein by reference). The first gene in the pTrc99A::budB-ilvC-ilvD-kivD isobutanol operon is budB encoding acetolactate synthase from Klebsiella pneumoniae ATCC 25955, followed by the ilvC gene encoding acetohydroxy acid reductoisomerase from E. coli. This is followed by ilvD encoding acetohydroxy acid dehydratase from E. coli and lastly the kivD gene encoding the branched-chain keto acid decarboxylase from L. lactis.

The sadB coding region was amplified from pTrc99a::sadB using primers N695A (SEQ ID NO: 233) and N696A (SEQ ID NO: 234) with Phusion High Fidelity DNA Polymerase (New England Biolabs, Beverly, Mass.). Amplification was carried out with an initial denaturation at 98 C. for 1 min, followed by 30 cycles of denaturation at 98° C. for 10 sec, annealing at 62° C. for 30 sec, elongation at 72° C. for 20 sec and a final elongation cycle at 72° C. for 5 min, followed by a 4° C. hold. Primer N695A contained an AvrII restriction site for cloning and a RBS upstream of the ATG start codon of the sadB coding region. The N696A primer included an XbaI site for cloning. The 1.1 kb PCR product was digested with AvrII and XbaI (New England Biolabs, Beverly, Mass.) and gel purified using a Qiaquick Gel Extraction Kit (Qiagen Inc., Valencia, Calif.)). The purified fragment was ligated with pTrc99A::budB-ilvC-ilvD-kivD, that had been cut with the same restriction enzymes, using T4 DNA ligase (New England Biolabs, Beverly, Mass.). The ligation mixture was incubated at 16° C. overnight and then transformed into E. coli Mach 1™ competent cells (Invitrogen) according to the manufacturer's protocol. Transformants were obtained following growth on the LB agar with 100 μg/ml ampicillin. Plasmid DNA from the transformants was prepared with QIAprep Spin Miniprep Kit (Qiagen Inc., Valencia, Calif.) according to manufacturer's protocols. The resulting plasmid was called pTrc99A::budB-ilvC-ilvD-kivD-sadB.

Electrocompetent cells of the strains listed in Table 8 were prepared as described and transformed with pTrc99A::budB-ilvC-ilvD-kivD-sadB (“pBCDDB”). Transformants were streaked onto LB agar plates containing 100 μg/mL ampicillin.

### Example 15

### Construction of an E. coli Production Host Containing an Isobutanol Biosynthetic Pathway and an Overexpression Plasmid Containing edp3-edp4

A DNA fragment encoding Phosphogluconate dehydratase (EC 4.2.1.12) (6-phosphogluconate dehydratase, (edp3)) (DNA SEQ ID NO: 139; protein SEQ ID NO: 140) and 2-dehydro-3-deoxy-phosphogluconate aldolase, (edp4) (EC 4.1.2.14) (protein SEQ ID NO: 209) (DNA SEQ ID NO 208 (edd-eda operon)) from E. coli MG1655 was amplified from E. coli genomic DNA using standard conditions. The DNA was prepared using a Gentra Puregene kit (Gentra Systems, Inc., Minneapolis, Minn.; catalog number D-5500A) following the recommended protocol for gram negative organisms. PCR amplification was done using forward and reverse primers EE F and EE R (SEQ ID NOs:317 and 318), respectively with Phusion High Fidelity DNA Polymerase (New England Biolabs, Beverly, Mass.). The forward primer incorporated an optimized E. coli RBS and a HindIII restriction site. The reverse primer included an XbaI restriction site. The 2.5 kb PCR product was cloned into pCR® 4Blunt-TOPO® (Invitrogen Corp. (Carlsbad, Calif.) to produce pCR4Blunt::edd-eda (edp3-edp4). The plasmid was transformed into E. coli Top10 cells. Plasmids from three clones were sequenced with primers EE Seq F2 (SEQ ID NO: 319) EE Seq F4 (SEQ ID NO: 320), EE Seq R4 (SEQ ID NO: 321) and EE Seq R3 (SEQ ID NO: 322) and the sequence verified.

The edd-eda coding region was then cloned into the vector pCL1925 (described in U.S. Pat. No. 7,074,608), a low copy plasmid carrying the glucose isomerase promoter from Streptomyces. The pCR4Blunt::edd-eda was digested with HindIII and XbaI and the 2.5 kb edd-eda fragment gel purified. The vector pCL1925 was cut with HindIII and XbaI and the 4.5 kb vector fragment gel purified. The edd-eda fragment was ligated with the pCL1925 vector fragment using T4 DNA ligase (New England Biolabs, Beverly, Mass.). The ligation mixture was incubated at 16° C. overnight and then transformed into E. coli Top10 cells creating pCL1925-edp3-edp4 (pED). Transformants were plated onto LB agar containing 50 μg/ml spectinomycin. A transformant was grown in LB 50 μg/ml spectinomycin and plasmid prepared using the QIAprep Spin Miniprep kit (Qiagen, Valencia, Calif.) according to manufacturer's recommendation.

The strain 8KO fbaB::kan containing pTrc99A::budB-ilvC-ilvD-kivD-sadB (pBCDDB) was made electrocompetent as previously described and transformed with pCL1925-edp3-edp4, also named “pED”. Transformants were plated onto LB agar containing 50 μg/ml spectinomycin and 100 μg/mL ampicillin.

### Example 16

### Production of Isobutanol in E. coli with Diminished Oxidative Pentose Phosphate and/or EMP and Functional EDP

E. coli K12 strains “E. coli 3KO adhE::kan” (E. coli K12 ΔpflB ΔfrdB ΔldhA ΔadhE::kan), “E. coli 4KO gnd::kan” (E. coli K12 ΔpflB ΔfrdB ΔldhA ΔadhE Δgnd::kan), “E. coli 5KO pfkA::kan” (E. coli K12 ΔpflB ΔfrdB ΔldhA ΔadhE Δgnd ΔpfkA::kan), “E. coli 6KO pfkB::kan” (E. coli K12 ΔpflB ΔfrdB ΔldhA ΔadhE Δgnd ΔpfkA ΔpfkB::kan), “E. coli 7KO fbaA::kan” (E. coli K12 ΔpflB ΔfrdB ΔldhA ΔadhE Δgnd ΔpfkA ΔpfkB ΔfbaA::kan), “E. coli 8KO fbaB::kan” (E. coli K12 ΔpflB ΔfrdB ΔldhA ΔadhE Δgnd ΔpfkA ΔpfkB ΔfbaA ΔfbaB::kan) were constructed as described in Example 13 in E. coli K-12 BW25113 and transformed with the isobutanol pathway plasmid, “+pBCDDB”. Additionally strain “E. coli 8KO fbaB::kan+pBCDDB” was also transformed with an overexpression plasmid “pED” containing “edp3” and “edp4” (pCL1925-edp3-edp4), described in more detail in Example 15, creating “E. coli 8KO fbaB::kan+pBCDDB+pED”. The Keio collection host strain, E. coli K-12 BW25113 was transformed with pTrc99a as an empty vector control, creating “E. coli BW25113+pTrc”.

Frozen glycerol stock cultures of the strains were generated by inoculating a single colony from selective antibiotic LB plates into 100 ml baffled Erlenmeyer shake flasks, filled with 20 ml LB medium and 100 μg/ml carbenicillin. Additionally 50 μg/ml spectinomycin had been added to the E. coli 8KO fbaB::kan+pBCDDB+pED culture. When the cultures reached an optical density of approximately 1.000 at λ=600 nm, 0.7 ml portions of the respective culture were transferred into 2 ml cryogenic vials (Nalgene, Rochester, N.Y.), 0.3 ml of sterile glycerol added, the cap closed and the vial vortexed for about 20 seconds. Subsequently the vials were immediately stored in the freezer at −80° C.

10 μl of frozen glycerol stocks from strains E. coli BW25113+pTrc, E. coli 3KO adhE::kan+pBCDDB, E. coli 4KO gnd::kan+pBCDDB, E. coli 5KO pfkA::kan+pBCDDB, E. coli 6KO pfkB::kan+pBCDDB, E. coli 7KO fbaA::kan+pBCDDB, E. coli 8KO fbaB::kan+pBCDDB and 15 μl of frozen glycerol stock from strain E. coli 8KO fbaB::kan+pBCDDB+pED were each inoculated into 15 ml culture tubes filled with 3.5 ml LB medium and 100 μg/ml carbenicillin. Additionally 50 μg/ml spectinomycin had been added to the E. coli 8KO fbaB::kan+pBCDDB+pED culture. The aerobic cultures were incubated over night at 30° C. and 250 rpm in an Innova Laboratory Shaker (New Brunswick Scientific, Edison, N.J.).

The next day 0.26 ml of the overnight culture from strain E. coli BW25113+pTrc, 0.28 ml of the overnight culture from strain E. coli 3KO adhE::kan+pBCDDB, 0.28 ml of the overnight culture from strain E. coli 4KO gnd::kan+pBCDDB, 0.30 ml of the overnight culture from strain E. coli 5KO pfkA::kan+pBCDDB, 0.30 ml of the overnight culture from strain E. coli 6KO pfkB::kan+pBCDDB, 0.32 ml of the overnight culture from strain E. coli 7KO fbaA::kan+pBCDDB, 0.30 ml of the overnight culture from strain E. coli 8KO fbaB::kan+pBCDDB and 0.30 ml of the overnight culture from strain E. coli 8KO fbaB::kan+pBCDDB+pED were transferred under anaerobic conditions (anaerobic chamber from Coy Laboratory Products, Grass Lake, Mich.) into 25 ml Balch tubes filled with 12 ml growth medium. For each strain 4 cultures (n =4) were inoculated and analyzed accordingly, with the exception of strain E. coli 3KO adhE::kan+pBCDDB, for which only 3 cultures (n=3) were inoculated and analyzed.

Initial optical densities at λ=600 nm measured with an Ultrospec 3000 spectrophotometer (Pharmacia Biotech, Piscataway, N.J.) were in average 0.144±0.005, 0.084±0.002, 0.088±0.007, 0.090±0.004, 0.099±0.001, 0.099±0.002, 0.104±0.002 and 0.093±0.002, respectively (see Table 8). The growth medium consisted of LB medium with about 20 g/l glucose, 0.1 M MOPs buffer at pH=7.0 and 100 μg/ml carbenicillin added. Growth medium of strain E. coli 8KO fbaB::kan+pBCDDB+pED contained in addition 50 μg/ml spectinomycin. Each Balch tube was fitted with a butyl rubber septum which allowed periodic gas and liquid sampling via syringe. The stopper was cramped to the tube with a sheet metal with circular opening on top for sampling with a syringe with needle through the rubber septum. The tubes were fixed at an angle of about 60° relative to the shaker plate and incubated in an Innova Laboratory Shaker (New Brunswick Scientific, Edison, N.J.) at 37° C. and 250 rpm.

In one of the cultivations of each strain initial concentrations of glucose, succinic acid, lactic acid, glycerol, acetic acid, ethanol and isobutanol were analyzed by HPLC as described previously and results are provided in Table 7a and 7b, “n.d.” indicates that the respective compound was not detected. Samples of each of the cultivations were withdrawn at 24 h and 48 h of the process and analyzed accordingly. Results are provided in Tables 9a and 9b. First number in a compound column is the average measured concentration value c(av) from quatriplicate (strain E. coli 3KO adhE::kan+pBCDDB: triplicate) experiments, the second indicates the standard deviation SD found in these experiments.

Also average optical density ODav from quatriplicate experiments (strain E. coli 3KO adhE::kan+pBCDDB: triplicate) as an indicator for the biomass dry weight concentration was analyzed for the strains not only at process time t=0 h, but also at t=24 h and t=48 h and is provided in Table 8, together with the standard deviation SD.

Table 10 shows the yields of isobutanol, Y(isobutanol, defined as the absolute difference of isobutanol concentrations measured at the beginning and the end of the 48 h experiment in [g/l], divided by the absolute difference of the glucose concentrations measured in [g/l] at Oh and 48 h of the experiments. Average values from the quatriplicate (strain E. coli 3KO adhE::kan+pBCDDB: triplicate) experiments between 0.31 g/g (E. coli 4KO gnd::kan+pBCDDB) and 0.40 g/g (E. coli 6KO pfkB::kan+pBCDDB) were achieved with the isobutanol producing strains. Maximum stoichiometric yield assuming 100% conversion of glucose through EDP with the given isobutanol pathway is 0.41 g/g. Thus, the isobutanol yields ranged from greater than about 75% of theoretical to over 95% of theoretical. Average standard deviations of the yield values, SD(Y), were calculated from the quatriplicate (strain E. coli 3KO adhE::kan+pBCDDB: triplicate) experiments applying error propagation to the averaged input values (see Table 10).

Also shown in Table 10 is the average volumetric productivity Qp (48) determined for the different strain cultivations after 48 h from the quatriplicate (strain E. coli 3KO adhE::kan+pBCDDB: triplicate) experiments. Average volumetric productivity was calculated as the absolute difference of the average isobutanol concentrations measured at the beginning and the end of the experiment in [mmol/l], divided by the time of the cultivation, 48 h. Average volumetric productivities were found to be between 0.16 mmol/1 h (E. coli 3KO adhE::kan+pBCDDB) and 0.50 mmol/1 h (E. coli 5KO pfkA::kan+pBCDDB) (see table 10). Average standard deviations of the average volumetric productivity, SD(Qp), were calculated from the quatriplicate (strain E. coli 3KO adhE::kan+pBCDDB: triplicate) experiments applying error propagation to the averaged input values.

TABLE 7a
Initial Product Concentrations

| | | Succinic | Lactic | | Acetic |
|---|---|---|---|---|---|
| | Glucose | acid | acid | Glycerol | acid |
| E. coli strain | [mM] | [mM] | [mM] | [mM] | [mM] |
| BW25113 + | 106.8 | 0.82 | 0.45 | 0.38 | 1.40 |
| pTrc | | | | | |
| 3KO adh::kan + | 106.4 | 0.72 | 0.46 | 0.41 | 1.19 |
| pBCDDB | | | | | |
| 4KO gnd::kan + | 106.5 | 0.73 | 0.46 | 0.42 | 1.27 |
| pBCDDB | | | | | |
| 5KO pfkA::kan + | 106.2 | 0.72 | 0.47 | 0.42 | 1.23 |
| pBCDDB | | | | | |
| 6KO pfkB::kan + | 105.7 | 0.72 | 0.43 | 0.39 | 1.25 |
| pBCDDB | | | | | |
| 7KO fbaA::kan + | 106.3 | 0.71 | 0.43 | 0.40 | 1.35 |
| pBCDDB | | | | | |
| 8KO fbaB::kan + | 106.3 | 0.70 | 0.44 | 0.42 | 1.39 |
| pBCDDB | | | | | |
| 8KO fbaB::kan + | 106.1 | 0.71 | 0.44 | 0.49 | 1.25 |
| pBCDDB + pED | | | | | |

TABLE 7b
Initial Product Concentrations

| | | | Ketoisovaleric | |
|---|---|---|---|---|
| | Ethanol | Pyruvic acid | acid | iso-Butanol |
| E. coli strain | [mM] | [mM] | [mM] | [mM] |
| BW25113 + | 2.54 | 0.25 | n.d. | n.d. |
| pTrc | | | | |
| 3KO adh::kan + | n.d. | 0.10 | n.d. | n.d. |
| pBCDDB | | | | |
| 4KO gnd::kan + | n.d. | 0.10 | n.d. | n.d. |
| pBCDDB | | | | |
| 5KO pfkA::kan + | n.d. | 0.12 | n.d. | n.d. |
| pBCDDB | | | | |
| 6KO pfkB::kan + | n.d. | 0.18 | n.d. | n.d. |
| pBCDDB | | | | |
| 7KO fbaA::kan + | n.d. | 0.20 | n.d. | n.d. |
| pBCDDB | | | | |
| 8KO fbaB::kan + | n.d. | 0.18 | n.d. | n.d. |
| pBCDDB | | | | |
| 8KO fbaB::kan + | n.d. | 0.14 | n.d. | n.d. |
| pBCDDB + pED | | | | |

TABLE 8
OD during the experiments

| | samples t = 0 h | | samples t = 24 h | | samples t = 48 h | |
|---|---|---|---|---|---|---|
| E. coli strain | ODav [ ] | SD [ ] | ODav [ ] | SD [ ] | ODav [ ] | SD [ ] |
| BW25113 + | 0.144 | 0.005 | 3.354 | 0.041 | 3.304 | 0.041 |
| pTrc | | | | | | |
| 3KO adh::kan + | 0.084 | 0.002 | 0.218 | 0.006 | 0.188 | 0.004 |
| pBCDDB | | | | | | |
| 4KO gnd::kan + | 0.088 | 0.007 | 0.374 | 0.008 | 0.312 | 0.008 |
| pBCDDB | | | | | | |
| 5KO pfkA::kan + | 0.090 | 0.004 | 0.563 | 0.032 | 0.612 | 0.050 |
| pBCDDB | | | | | | |
| 6KO pfkB::kan + | 0.099 | 0.001 | 0.543 | 0.023 | 0.703 | 0.030 |
| pBCDDB | | | | | | |
| 7KO fbaA::kan + | 0.099 | 0.002 | 0.510 | 0.011 | 0.657 | 0.018 |
| pBCDDB | | | | | | |
| 8KO fbaB::kan + | 0.104 | 0.002 | 0.562 | 0.013 | 0.799 | 0.009 |
| pBCDDB | | | | | | |
| 8KO fbaB::kan + | 0.093 | 0.002 | 0.345 | 0.021 | 0.511 | 0.044 |
| pBCDDB + pED | | | | | | |

TABLE 9a
Products from Fermentation with Microbial Host Cells

| | | | | | Succinic | | Lactic | | | |
|---|---|---|---|---|---|---|---|---|---|---|
| | | | Glucose | | acid | | acid | | Glycerol | |
| | | | [mM] | | [mM] | | [mM] | | [mM] | |
| Cultures | | Sample | c (av) | SD | c (av) | SD | c (av) | SD | c (av) | SD |
| E. coli | BW25113 + | 24 h | 59.3 | 0.2 | 6.4 | 0.0 | 50.1 | 0.2 | 0.3 | 0.0 |
| | pTrc | | | | | | | | | |
| | | 48 h | 59.3 | 0.2 | 6.4 | 0.0 | 50.1 | 0.3 | 0.4 | 0.1 |
| E. coli | 3KO adh::kan + | 24 h | 98.5 | 0.1 | 1.0 | 0.0 | 0.7 | 0.0 | 0.5 | 0.0 |
| | pBCDDB | | | | | | | | | |
| | | 48 h | 98.2 | 0.2 | 1.2 | 0.0 | 0.7 | 0.0 | 0.5 | 0.0 |
| E. coli | 4KO gnd::kan + | 24 h | 94.6 | 0.5 | 1.8 | 0.0 | 0.8 | 0.0 | 0.4 | 0.0 |
| | pBCDDB | | | | | | | | | |
| | | 48 h | 92.5 | 0.1 | 0.0 | 0.0 | 1.0 | 0.0 | 0.4 | 0.0 |
| E. coli | 5KO pfkA::kan + | 24 h | 94.0 | 0.8 | 1.2 | 0.0 | 0.5 | 0.1 | 0.0 | 0.0 |
| | pBCDDB | | | | | | | | | |
| | | 48 h | 81.0 | 1.7 | 1.4 | 0.1 | 0.7 | 0.0 | 0.0 | 0.0 |
| E. coli | 6KO pfkB::kan + | 24 h | 95.6 | 0.3 | 1.2 | 0.0 | 0.7 | 0.0 | 0.0 | 0.0 |
| | pBCDDB | | | | | | | | | |
| | | 48 h | 84.1 | 0.7 | 1.3 | 0.0 | 0.7 | 0.1 | 0.0 | 0.0 |
| E. coli | 7KO fbaA::kan + | 24 h | 96.2 | 0.2 | 1.2 | 0.1 | 0.7 | 0.0 | 0.0 | 0.0 |
| | pBCDDB | | | | | | | | | |
| | | 48 h | 85.3 | 0.3 | 1.3 | 0.1 | 0.7 | 0.0 | 0.0 | 0.0 |
| E. coli | 8KO fbaB::kan + | 24 h | 95.8 | 0.3 | 1.1 | 0.0 | 0.7 | 0.0 | 0.2 | 0.0 |
| | pBCDDB | | | | | | | | | |
| | | 48 h | 86.1 | 0.1 | 1.3 | 0.0 | 0.6 | 0.0 | 0.0 | 0.0 |
| E. coli | 8KO fbaB::kan + | 24 h | 99.2 | 0.6 | 0.9 | 0.0 | 0.7 | 0.0 | 0.4 | 0.0 |
| | pBCDDB + | | | | | | | | | |
| | pED | | | | | | | | | |
| | | 48 h | 93.2 | 0.8 | 1.0 | 0.0 | 0.7 | 0.0 | 0.3 | 0.0 |

TABLE 9b
Products from Fermentation with Microbial Host Cells

| | | | Acetic | | | | Pyruvic | | Ketoisovaleric | | Iso- | |
|---|---|---|---|---|---|---|---|---|---|---|---|---|
| | | | acid | | Ethanol | | acid | | acid | | Butanol | |
| | | | [mM] | | [mM] | | [mM] | | [mM] | | [mM] | |
| Cultures | | Sample | c (av) | SD | c (av) | SD | c (av) | SD | c (av) | SD | c (av) | SD |
| E. coli | BW25113 + | 24 h | 21.9 | 0.1 | 22.5 | 0.3 | 0.3 | 0.0 | 0.0 | 0.0 | 0.0 | 0.0 |
| | pTrc | | | | | | | | | | | |
| | | 48 h | 21.7 | 0.1 | 22.6 | 0.3 | 0.4 | 0.0 | 0.0 | 0.0 | 0.0 | 0.0 |
| E. coli | 3KO adh::kan + | 24 h | 1.2 | 0.1 | 0.0 | 0.0 | 0.4 | 0.0 | 0.5 | 0.0 | 7.2 | 0.1 |
| | pBCDDB | | | | | | | | | | | |
| | | 48 h | 1.3 | 0.1 | 0.0 | 0.0 | 0.5 | 0.0 | 0.5 | 0.0 | 7.9 | 0.2 |
| E. coli | 4KO gnd::kan + | 24 h | 1.4 | 0.0 | 0.0 | 0.0 | 0.5 | 0.0 | 0.4 | 0.0 | 8.6 | 0.4 |
| | pBCDDB | | | | | | | | | | | |
| | | 48 h | 1.1 | 0.2 | 0.0 | 0.0 | 0.6 | 0.0 | 0.3 | 0.2 | 10.8 | 0.3 |
| E. coli | 5KO pfkA::kan + | 24 h | 1.8 | 0.0 | 0.0 | 0.0 | 0.6 | 0.0 | 0.4 | 0.0 | 11.2 | 0.6 |
| | pBCDDB | | | | | | | | | | | |
| | | 48 h | 2.5 | 0.1 | 0.0 | 0.0 | 0.6 | 0.0 | 0.7 | 0.1 | 24.2 | 1.5 |
| E. coli | 6KO pfkB::kan + | 24 h | 2.3 | 0.0 | 0.0 | 0.0 | 0.3 | 0.0 | 0.4 | 0.0 | 9.9 | 0.5 |
| | pBCDDB | | | | | | | | | | | |
| | | 48 h | 3.3 | 0.1 | 0.0 | 0.0 | 0.4 | 0.0 | 0.9 | 0.0 | 21.1 | 0.8 |
| E. coli | 7KO fbaA::kan + | 24 h | 2.4 | 0.1 | 0.0 | 0.0 | 0.4 | 0.0 | 0.5 | 0.1 | 9.0 | 0.2 |
| | pBCDDB | | | | | | | | | | | |
| | | 48 h | 3.2 | 0.2 | 0.0 | 0.0 | 0.4 | 0.0 | 1.0 | 0.1 | 19.7 | 0.3 |
| E. coli | 8KO fbaB::kan + | 24 h | 2.3 | 0.1 | 0.0 | 0.0 | 0.3 | 0.0 | 0.5 | 0.0 | 9.3 | 0.3 |
| | pBCDDB | | | | | | | | | | | |
| | | 48 h | 3.3 | 0.1 | 0.0 | 0.0 | 0.4 | 0.0 | 0.7 | 0.0 | 19.1 | 0.1 |
| E. coli | 8KO fbaB::kan + | 24 h | 2.3 | 0.0 | 0.0 | 0.0 | 0.5 | 0.0 | 0.5 | 0.0 | 6.3 | 0.4 |
| | pBCDDB + pED | | | | | | | | | | | |
| | | 48 h | 3.2 | 0.1 | 0.0 | 0.0 | 0.4 | 0.1 | 0.7 | 0.1 | 12.3 | 0.7 |

TABLE 10
Yield and Average Volumetric Productivity

| | | Y (iso- | | | |
|---|---|---|---|---|---|
| | | Butanol) | SD (Y) | Qp (48) | SD (Qp) |
| Cultures | | [g/g] | [g/g] | [mmol/l h] | [mmol/l h] |
| E. coli | BW25113 + | 0.00 | 0.00 | 0.00 | 0.00 |
| | pTrc | | | | |
| E. coli | 3KO adh::kan + | 0.39 | 0.00 | 0.16 | 0.00 |
| | pBCDDB | | | | |
| E. coli | 4KO gnd::kan + | 0.31 | 0.01 | 0.23 | 0.01 |
| | pBCDDB | | | | |
| E. coli | 5KO pfkA::kan + | 0.39 | 0.00 | 0.50 | 0.01 |
| | pBCDDB | | | | |
| E. coli | 6KO pfkB::kan + | 0.40 | 0.00 | 0.44 | 0.01 |
| | pBCDDB | | | | |
| E. coli | 7KO fbaA::kan + | 0.39 | 0.01 | 0.41 | 0.00 |
| | pBCDDB | | | | |
| E. coli | 8KO fbaB::kan + | 0.39 | 0.00 | 0.40 | 0.01 |
| | pBCDDB | | | | |
| E. coli | 8KO fbaB::kan + | 0.39 | 0.00 | 0.26 | 0.01 |
| | pBCDDB + pED | | | | |

### Example 17

### Prophetic

### ¹³C Tracer Analysis to Demonstrate Isobutanol Production with a Functional and/or Enhanced ED Pathway in E. coli

Strains E. coli BW25113+pTrc (E. coli K-12 BW25113+pTrc99a), E. coli 3KO adhE::kan+pBCDDB (E. coli K12 ΔpflB ΔfrdB ΔldhA ΔadhE::kan+pBCDDB), E. coli 4KO gnd::kan+pBCDDB (E. coli K12 ΔpflB ΔfrdB ΔldhA ΔadhE Δgnd::kan+pBCDDB), E. coli 5KO pfkA::kan+pBCDDB (E. coli K12 ΔpflB ΔfrdB ΔldhA ΔadhE Δgnd ΔpfkA::kan+pBCDDB), E. coli 6KO pfkB::kan

+pBCDDB (E. coli K12 ΔpflB ΔfrdB ΔldhA ΔadhE Δgnd ΔpfkA ΔpfkB::kan+pBCDDB), E. coli 7KO fbaA::kan+pBCDDB (E. coli K12 ΔpflB ΔfrdB ΔldhA ΔadhE Δgnd ΔpfkA ΔpfkB ΔfbaA::kan+pBCDDB), E. coli 8KO fbaB::kan+pBCDDB (E. coli K12 ΔpflB ΔfrdB ΔldhA ΔadhE Δgnd ΔpfkA ΔpfkB ΔfbaA ΔfbaB::kan+pBCDDB) and E. coli 8KO fbaB::kan+pBCDDB+pED (E. coli K12 ΔpflB ΔfrdB ΔldhA ΔadhE Δgnd ΔpfkA ΔpfkB ΔfbaA ΔfbaB::kan+pBCDDB+pCL1925-edp3-edp4) are constructed as described previously and stored as frozen glycerol stock cultures.

10 μl of frozen glycerol stocks from strains E. coli BW25113+pTrc, E. coli 3KO adhE::kan+pBCDDB, E. coli 4KO gnd::kan+pBCDDB, E. coli 5KO pfkA::kan+pBCDDB, E. coli 6KO pfkB::kan+pBCDDB, E. coli 7KO fbaA::kan+pBCDDB, E. coli 8KO fbaB::kan+pBCDDB and E. coli 8KO fbaB::kan+pBCDDB+pED are inoculated into 15 ml culture tubes filled with 3.5 ml LB medium and 100 μg/ml carbenicillin. Additionally 50 μg/ml spectinomycin are added to the E. coli 8KO fbaB::kan+pBCDDB+pED culture. The aerobic cultures are incubated over night at 30° C. and 250 rpm in an Innova Laboratory Shaker (New Brunswick Scientific, Edison, N.J.).

The next day 250 μl of each culture are transferred under anaerobic conditions into 25 ml Balch tubes filled with 12 ml growth medium. Initial optical densities are measured at λ=600 nm. The growth medium consists of LB medium with 100 mM glucose, 0.1 M MOPs buffer at pH=7.0 and 100 μg/ml carbenicillin added. Growth medium of strain E. coli 8KO fbaB::kan+pBCDDB+pED contains in addition 50 μg/ml spectinomycin.

Carbon naturally occurs in two major stable isotopes, ¹²C and ¹³C, at an abundancy of about 98.9% and 1.1%. The naturally occurring ratio of ¹²C/¹³C is called “natural abundance”. The glucose in the ¹³C tracer experiment consists out of approximately 40% glucose labeled at natural abundance, 40% glucose with a ¹³C atom at the C1 position of the molecule, and 20% of fully labeled ¹³C glucose.

Each Balch tube is fitted with a butyl rubber septum which allowed periodic gas and liquid sampling via syringe. The stopper is cramped to the tube with a sheet metal with circular opening on top for sampling with a syringe with needle through the rubber septum.

Samples are withdrawn at 0 h, 24 h and 48 h of the process and analyzed for their concentrations of glucose, succinic acid, lactic acid, glycerol, acetic acid, ethanol and isobutanol by HPLC as described previously. Isobutanol formation is detected. Optical densities at λ=600 nm are determined at 0 h, 24 h and 48 h and biomass growth is determined.

Samples are spun down with an Eppendorf centrifuge at 14.000 rpm and 2 min, the supernatant is retained and the pellet discarded. For volatile analysis, the supernatant is used directly. For analysis of non-volatile compounds, 400 μL of the supernatant is dried under vacuum in a speedvac at 45° C. Dried material is resuspended in 100 μl of Methoxyamine.HCl in Pyridine (Sigma-Aldrich, St. Louis, Mo.) and 100 μl of N-(tert-butyldimethylsilyl)-N-methyl-trifluoroacetamide (MTBSTFA) (Sigma-Aldrich, St. Louis, Mo.) is added. The mixture is incubated for 60 min at 60° C.

For analysis of proteinogenic amino acids, cell pellets equivalent to 4-8 mg of dry weight (if necessary, replica experiments can be pooled) are dissolved in 1.5 ml of 6 N HCl and incubated for 24 h at 110° C. in a heating block. The hydrolyzates are dried under vacuum in a speedvac at 45° C. For derivatization, the dried hydrolyzates is resuspended in 100 μl of 2% Methoxyamine.HCl in Pyridine (Sigma-Aldrich, St. Louis, Mo.) and 100 μl of N-(tert-butyldimethylsilyl)-N-methyl-trifluoroacetamide (MTBSTFA) (Sigma-Aldrich, St. Louis, Mo.) was added. The mixture is incubated for 60 min at 60° C., transferred to GC vials and injected into the GC/MS.

GC/MS analysis is carried out with a HP GC6890 equipped with a MSD5973 detector. In the analysis of TBDMS derivatives, a Supelco Equity-1 column (30 m×0.32 mm×0.25 m) is applied. The injection volume is 1 μL at a carrier gas flow of 2 mL/min helium with a split ratio of 1:20. The initial oven temperature of 150° C. is maintained for 2 min and then raised to 280° C. at 3 C/min. Other settings are 280° C. interface temperature, 200° C. ion source temperature, and electron impact ionization (EI) at 70 eV. Mass spectra are analyzed in the range of 100-660 atom mass units (amu) at a rate of 2.46 scans/sec for a run time of 45.33 min. Mass isotopomer distributions of non-volatile compounds and proteinogenic amino acids are determined.

Volatile compounds in supernatant are analyzed with a HP-INNOWAX polyethylene glycol column (30 m×0.25 mm×0.25 um). The injection volume is 0.5 μL at a carrier gas flow of 1 mL/min helium with a split ratio of 1:5. The initial oven temperature of 45° C. is maintained for 1 min, raised to 220° C. at a rate of 10° C./min and hold for another 5 min (total run time: 23.50 min). Other settings are 220° C. interface temperature, 250° C. ion source temperature, and electron impact ionization (EI) at 70 eV. Mass spectra are analyzed in the range of 40-350 atom mass units (amu) at a rate of 4.52 scans/sec. Mass isotopomer distributions of volatile compounds are determined.

Based on the results for the biomass, by-products and mass isotopomers measurements, flux through ED pathway is calculated either by metabolic flux ratio analysis, based on algebraic equations as exemplified in the art (Christensen, Christiansen et al. 2001, Biotechnol Bioeng 74(6): 517-523) or (Nanchen, Fuhrer et al. 2007, Methods Mol Biol 358: 177-197), or with the help of metabolic flux analysis, based on the balancing of mass isotopomers, as described in the art (Dauner, Bailey et al. 2001, Biotechnol Bioeng 76(2): 144-156), (Antoniewicz, Kelleher et al. 2007, Metab Eng 9(1): 68-86) or (Zamboni, Fendt et al. 2009, Nat Protoc 4(6): 878-892). In preferred embodiments, the relative flux through at least one reaction unique to the EDP is at least 1% greater than that in the control host, demonstrating that isobutanol is produced with the help of a functional and/or enhanced ED pathway. In other preferred embodiments, the relative flux through at least one reaction unique to the EDP is at least about 10% 50%, or 90% greater than that in the control host. In other embodiments, the relative flux through at least one reaction unique to the EMP or PPP is at least about 1% less than that in the control host, demonstrating that isobutanol is produced with the help of a functional and/or enhanced ED pathway. In other embodiments, the combined relative flux through the EMP and PPP is at least about 1% less than that in the control host, demonstrating that isobutanol is produced with the help of a functional and/or enhanced ED pathway.

1. A recombinant microbial host cell comprising a functional or enhanced EDP and an isobutanol production pathway wherein said functional or enhanced EDP provides for increased isobutanol production as compared to the same host cell without said functional or enhanced EDP.

2. The microbial host cell of claim 1 wherein the functional or enhanced EDP is provided by expression of one or more heterologous genes that encode functional EDP pathway enzymes or up-regulation of one or more endogenous genes that encode enhanced EDP pathway enzymes, or both, and one or more modification to said host cell that provides for increased carbon flux through the EDP or reducing equivalents balance such that the cofactors produced during the conversion of glucose to pyruvate are matched with the cofactors required for the conversion of pyruvate to isobutanol, or both, whereby isobutanol production is increased as compared to the same host cell without said one or more modification that provides for increased carbon flux through the EDP or reducing equivalents balance, or both.

3. The microbial host cell of claim 2 wherein said one or more modification to said host cell that provides for increased carbon flux through EDP or reducing equivalents balance, or both, is one or more genetic modification selected from the group consisting of:
a) a disruption in the expression of at least one enzyme of the EMP;
b) a disruption in the expression of at least one enzyme of the PPP; and
c) a modification in any one of EDP, EMP, or PPP such that cofactors produced during the conversion of glucose to pyruvate are matched with the cofactors required for the conversion of pyruvate to isobutanol.

4. The microbial host cell of claim 1, wherein said host cell comprises:
i) at least one gene encoding acetolactate synthase for the conversion of pyruvate to acetolactate;
ii) at least one gene encoding ketol acid reductoisomerase for the conversion of acetolactate to 2,3-dihydroxyisovalerate;
iii) at least one gene encoding an acetohydroxy acid dehydratase for the conversion of 2,3-dihydroxyisovalerate to α-ketoisovalerate;
iv) at least one gene encoding valine dehydrogenase or transaminase for the conversion of α-ketoisovalerate to valine;
v) at least one gene encoding a valine decarboxylase for the conversion of valine to isobutylamine;
vi) at least one gene encoding an omega transaminase for the conversion of isobutylamine to isobutyraldehyde; and
vii) at least one gene encoding a branched chain alcohol dehydrogenase for the conversion of isobutyraldehyde to isobutanol.

5. The microbial host cell of claim 1 wherein said host cell comprises:
i) at least one gene encoding acetolactate synthase for the conversion of pyruvate to acetolactate;
ii) at least one gene encoding ketol acid reductoisomerase for the conversion of acetolactate to 2,3-dihydroxyisovalerate;
iii) at least one gene encoding acetohydroxy acid dehydratase for the conversion of 2,3-dihydroxyisovalerate to α-ketoisovalerate;
iv) at least one gene encoding a branched chain ketoacid dehydrogenase for the conversion of α-ketoisovalerate to isobutyryl-CoA;
v) at least one gene encoding an acylating aldehyde dehydrogenase for the conversion of isobutyryl-CoA to isobutyraldehyde;, and
vi) at least one gene encoding a branched chain aldehyde dehydrogenase for the conversion of isobutyraldehyde to isobutanol.

6. The microbial host cell of claim 1, wherein said host cell comprises:
i) at least one gene encoding acetolactate synthase for the conversion of pyruvate to acetolactate;
ii) at least one gene encoding acetohydroxy acid reductoisomerase for the conversion of acetolactate to 2,3-dihydroxyisovalerate;
iii) at least one gene encoding acetohydroxy acid dehydratase for the conversion of 2,3-dihydroxyisovalerate to α-ketoisovalerate;
iv) at least one gene encoding branched-chain a-keto acid decarboxylase for the conversion of α-ketoisovalerate to isobutyraldehyde; and
v) at least one gene encoding branched-chain alcohol dehydrogenase for the conversion of isobutyraldehyde to isobutanol.

7. The microbial host cell of claim 1 wherein the functional or enhanced EDP is provided by expression of at least one recombinant DNA molecule encoding an enzyme of the EDP selected from the group consisting of
a) glucose-6-phosphate dehydrogenase;
b) 6-phosphogluconolactonase;
c) phosphogluconate dehydratase; and
d) 2-dehydro-3-deoxyphosphogluconate aldolase.

8. The microbial host cell of claim 3 wherein said disruption in expression of at least one enzyme of the EMP is a disruption in expression of at least one enzyme selected from the group consisting of:
a) 6-phosphofructokinase;
b) fructose-bisphosphate aldolase; and
c) glucose-6-phosphate isomerase.

9. The microbial host cell of claim 1 wherein the host cell is a member of the genera Clostridium, Zymomonas, Escherichia, Salmonella, Serratia, Erwinia, Klebsiella, Shigella, Rhodococcus, Pseudomonas, Bacillus, Lactobacillus, Enterococcus, Alcaligenes, Klebsiella, Paenibacillus, Arthrobacter, Corynebacterium, Brevibacterium, Schizosaccharomyces, Kluyveromyces, Yarrowia, Pichia, Candida, Hansenula, or Saccharomyces.

10. The microbial host cell of claim 1 wherein the host cell is E. coli, S. cerevisiae, or L. plantarum.

11. The microbial host cell of claim 9 wherein the host cell is E. coli and wherein the host cell further comprises downregulation or deletion of soluble transhydrogenase activity.

12. The microbial host cell claim 3 wherein the host cell comprises a disruption in at least one of the following genes:
pfk1, pfk2, fba1, gnd1, gnd2, pgi, pfkA, pfkB, fbaA, fbaB, gnd, pgi, sthA, PGI1, PFK1, PFK2, FBA1, GND1, or GND2.

13. A recombinant microbial host cell comprising an isobutanol production pathway and at least one of the following:
a) at least one recombinant DNA molecule encoding an enzyme of the EDP;
b) a disruption in the expression of at least one enzyme of the EMP; or
c) a disruption in the expression of at least one enzyme of the PPP; wherein production of isobutanol by said host cell is enhanced by at least 10% as compared to the same host cell without one of (a)-(c).

14-18. (canceled)

19. A method for the production of isobutanol comprising
a) providing the microbial host cell of claim 1; and
b) contacting the host cell with a fermentable carbon substrate under anaerobic conditions.

20. The method of claim 19 wherein the host cell is E. coli and wherein endogenous pyruvate formate lyase, fumarate reductase, alcohol dehydrogenase, and lactate dehydrogenase activities are downregulated or disrupted.

21. The method of claim 20 wherein the yield of isobutanol is greater than or equal to about 0.3 g/g.

22. The method of claim 20 wherein the yield of isobutanol is greater than or equal to about 0.35 g/g.

23. The method of claim 20 wherein the yield of isobutanol is greater than or equal to about 0.39 g/g.

24. The method of claim 19 wherein the host cell is S. cerevisiae and wherein endogenous pyruvate decarboxylase activity is downregulated or disrupted.

25. The method of claim 19 wherein the host cell is L. plantarum and wherein endogenous lactate dehydrogenase activity is downregulated or disrupted.
